(19) United States
(12) Patent Application Publication
(10) Pub. No.: US 2015/0004716 A1
(43) Pub. Date: Jan. 1, 2015

(54) Detection of Exposure to Chemical Warfare Nerve Agents with Lateral Flow Assays

(71) Applicant: Rapid Pathogen Screening, Inc., (US)

(72) Inventors: Robert P. Sambursky, Bradenton, FL (US); Robert W. VanDine, Montoursville, PA (US); Uma Mahesh Babu, Bradenton, FL (US); Peter Condon, Longboat Key, FL (US)

(73) Assignee: RAPID PATHOGEN SCREENING, INC., Sarasota, FL (US)

(21) Appl. No.: 13/796,560

(22) Filed: Mar. 12, 2013

Related U.S. Application Data
(63) Continuation-in-part of application No. 12/782,162, filed on May 18, 2010.
Application No. 12/782,162 is a continuation-in-part of application No. 12/469,207, filed on May 20, 2009, now abandoned.
Application No. 12/782,162 is a continuation-in-part of application No. PCT/US2009/057775, filed on Sep. 22, 2009.
Application No. PCT/US2009/057775 is a continuation-in-part of application No. 12/481,631, filed on Jun. 10, 2009, now Pat. No. 8,470,608.
Application No. 12/481,631 is a continuation-in-part of application No. 12/958,454, filed on Dec. 2, 2010, now Pat. No. 8,609,433.
Application No. 12/958,454 is a continuation-in-part of application No. 13/788,616, filed on Mar. 7, 2013, now Pat. No. 8,815,609.
(60) Provisional application No. 61/179,059, filed on May 18, 2009.
(60) Provisional application No. 61/071,833, filed on May 20, 2008.
(60) Provisional application No. 61/060,258, filed on Jun. 10, 2008.
(60) Provisional application No. 61/266,641, filed on Dec. 4, 2009.
(60) Provisional application No. 61/331,966, filed on May 6, 2010.
(60) Provisional application No. 61/352,093, filed on Jun. 7, 2010.
(60) Provisional application No. 61/392,981, filed on Oct. 14, 2010.

(51) Int. Cl. G01N 33/53

(52) U.S. Cl. CPC G01N 33/5308; G01N 2430/00
USPC 436/501; 422/69

(57) ABSTRACT

Rapid, point-of-care assays and methods detect clinically relevant organophosphate (OP) poisoning after low-level exposure to Sarin, Soman, Tabun, or VX chemical nerve agents. In some preferred embodiments, the assays are direct sandwich assays or direct correlation inhibition assays. The test preferably utilizes either a finger stick peripheral blood sample or plasma specimen.

### REFERENCE TO RELATED APPLICATIONS

This application is a continuation-in-part patent application of:

Co-pending application Ser. No. 12/782,162, filed May 18, 2010, entitled “METHOD AND DEVICE FOR COMBINED DETECTION OF VIRAL AND BACTERIAL INFECTIONS”, which claims one or more inventions which were disclosed in Provisional Application No. 61/179,059, filed May 18, 2009, entitled “METHOD AND DEVICE FOR COMBINED DETECTION OF VIRAL AND BACTERIAL INFECTIONS”, which is also a continuation-in-part application of application Ser. No. 12/469,207, filed May 20, 2009, entitled “NANOPARTICLES IN DIAGNOSTIC TESTS”, which claimed priority from Provisional Application No. 61/071,833, filed May 20, 2008, entitled “NANOPARTICLES IN DIAGNOSTIC TESTS” and PCT application Serial Number PCT/US2009/057775, filed Sep. 22, 2009, entitled “METHOD AND DEVICE FOR COMBINED DETECTION OF VIRAL AND BACTERIAL INFECTIONS”;

Co-pending application Ser. No. 12/481,631, filed Jun. 10, 2009, entitled “COMBINED VISUAL/FLUORESCENCE ANALYTE DETECTION TEST”, which claimed priority from Provisional Application No. 61/060,258, filed Jun. 10, 2008, entitled “COMBINED VISUAL/FLUORESCENCE ANALYTE DETECTION TEST”;

Co-pending application Ser. No. 12/958,454, filed Dec. 2, 2010, entitled “MULTIPLANAR LATERAL FLOW ASSAY WITH SAMPLE COMPRESSOR”, which claimed priority from Provisional Application No. 61/266,641, filed Dec. 4, 2009, entitled “LATERAL FLOW NUCLEIC ACID DETECTOR”, Provisional Application No. 61/331,966, filed May 6, 2010, entitled “MULTIPLANAR LATERAL FLOW ASSAY WITH SAMPLE COMPRESSOR”, Provisional Application No. 61/352,093, filed Jun. 7, 2010, entitled “LATERAL FLOW ASSAYS”, and Provisional Application No. 61/392,981, filed Oct. 14, 2010, entitled “MULTIPLANAR LATERAL FLOW ASSAY WITH SAMPLE COMPRESSOR”.

Co-pending application Ser. No. 13/788,616, filed Mar. 7, 2013, entitled “MULTIPLANAR LATERAL FLOW ASSAY WITH DIVERTING ZONE”.

The benefit under 35 USC §119(e) of the United States provisional applications are hereby claimed, and the aforementioned applications are hereby incorporated herein by reference.

### ACKNOWLEDGMENT OF GOVERNMENT SUPPORT

This invention was made with Government support under SBIR Grant No. W81XWH-06-C-0367, awarded by the Department of Defense. The government has certain rights in the invention.

### BACKGROUND OF THE INVENTION

1. Field of the Invention

The invention pertains to the field of lateral flow assays. More particularly, the invention pertains to detecting exposure to organophosphate chemical warfare nerve agents with lateral flow assays.

2. Description of Related Art

Chemical nerve agents pose a significant global security and health risk. These nerve agents target the enzyme acetylcholinesterase (AChE), which normally hydrolyses the neurotransmitter acetylcholine. Inhibition of AChE results in an excess of acetylcholine to remain within nerve synapses, causing over-stimulation of the cholinergic nervous system, paralysis, and ultimately death.

There is an unmet need to rapidly identify patients that have been exposed to low levels of Soman, Sarin, Tabun, or VX chemical nerve agents. Low level nerve agent exposure produces vague, nondescript signs and symptoms that are not easily differentiated from other conditions. The lack of an accurate diagnosis of low-level exposure to chemical nerve agents may result in a delay in, or lack of receipt of, needed treatment, the use of life-endangering resuscitation drugs, or the use of high risk anesthetics that can cause injury or death in those who have been exposed to chemical nerve agents.

In the event of a military or terrorist release of a chemical warfare nerve agent, the analysis of biomedical samples for the presence of biomarkers to confirm exposure is imperative to ensure that appropriate medical countermeasures are administered in a timely manner. In addition to identifying those with low level nerve agent poisoning, it is important to verify non-exposure to reassure worried civilian or military personnel.

After inhalation or contact with skin, nerve agents enter the blood and immediately combine with cholinesterases and other blood proteins including albumin, the most abundant serum protein, to form blood protein-nerve agent adducts. Nerve agents are unstable in pure aqueous solutions and are rapidly degraded on entering the body. Therefore, analysis for the presence of intact agents in blood is inappropriate unless samples are collected immediately after exposure.

The inhibition of AChE in red blood cells, or the related enzyme butyrylcholinesterase (BuChE) in serum and plasma, is the foundation of monitoring nerve agent exposure using the Ellman colorimetric/photometric method or modifications. In addition, two other methods to detect exposure to nerve agents have been utilized. The fluoride reactivation method is based on the principle that upon incubation of phosphylated binding with a large excess of fluoride ions, the phosphyl moiety is quantitatively converted into the corresponding phosphonofluoridate or phosphorofluoridate that can be isolated by solid-phase extraction and quantitated by gas chromatography with nitrogen phosphorus detection (GC-NPD) or gas chromatograph/mass spectrometer (GC/MS). Alternatively, mass spectrometric determination of specific nonapeptide adducts that result after pepsin digestion of modified BuChE may be utilized. A limitation of using mass spectrometric analysis to confirm nerve agent exposure is that it requires knowledge and advance input of the suspected specific type of organophosphate poison prior to testing.

Immediately upon entering the bloodstream, nerve agents causes an inhibition of BuChE. Clinical symptoms typically appear once BuChE has reached an inhibition level of approximately 85% following exposure to Sarin. To emulate an exposure, human plasma may be incubated with chemical nerve agents. During this incubation, concentrations of nerve agents from 1 to 25 ng/ml correlate with increasing BuChE inhibition from 10% to 100%. FIG. 1 shows the percentage of BuChE inhibition as it relates to levels at which symptoms become apparent. At 10 ng/ml exposure level, the inhibition of BuChE is measurable but clinical signs are not yet apparent. After an exposure leading to blood nerve agent levels of 10 ng/ml, BuChE will be substantially inhibited, and neurological function will become compromised, requiring immediate medical care and intervention. Therefore, the clinically relevant lower limit of detection of a qualitative, optically interpreted assay should be at a chemical nerve agent blood exposure level of 10 ng/ml. If an electronic reader is used to quantitate the results, the lower limit of detection should extend below the clinically relevant levels in order to provide a broader range of values for diagnostic interpretation.

### SUMMARY OF THE INVENTION

Rapid, point-of-care assays and methods detect clinically relevant organophosphate (OP) poisoning after low-level exposure to Sarin, Soman, Tabun, or VX chemical nerve agents. In some preferred embodiments, the assays are direct sandwich assays or direct correlation inhibition assays. The test preferably utilizes either a finger stick peripheral blood sample or plasma specimen.

### BRIEF DESCRIPTION OF THE DRAWINGS

FIG. 1 shows the relationship of BuChE levels and nerve agent exposure symptoms.

FIG. 2 shows the antibody nerve agent binding site.

FIG. 3A shows a diagram of Soman.

FIG. 3B shows a diagram of a first Soman analog used as an immunogen to produce anti-Soman antibodies.

FIG. 3C shows a diagram of a second Soman analog used as an immunogen to produce anti-Soman antibodies.

FIG. 4A shows an example of a negative test result.

FIG. 4B shows an example of a positive test result.

FIG. 5 shows the ID8.2 antibody.

FIG. 6A shows partial chemical structures of some chemical warfare nerve agents.

FIG. 6B shows the common architecture of the nerve agents of FIG. 6A around the phosphorus atom.

FIG. 7 shows adduct formation.

FIG. 8 shows the nerve agent binding site for antibody ID8.2.

FIG. 9 shows a sample analysis device in an embodiment of the present invention.

FIG. 10 shows a housing containing the strip of FIG. 9.

FIG. 11 shows a collection device for collecting a sample.

FIG. 12 shows a test kit including the sample analysis device of FIGS. 9 and 10 and the collection device of FIG. 11.

FIG. 13 shows another embodiment of a sample analysis device of the present invention.

FIG. 14 shows a device with a test line corresponding to the presence of a target in an embodiment of the present invention.

FIG. 15 shows a device with a test line corresponding to the presence of a target in another embodiment of the present invention.

FIG. 16 shows a test strip and a sample collector in a lateral flow device.

FIG. 17A shows a sample compressor in an embodiment of the present invention.

FIG. 17B shows another sample compressor in an embodiment of the present invention.

FIG. 17C shows a sample collector in an embodiment of the present invention.

FIG. 18A shows a lateral flow test strip in an embodiment of the present invention.

FIG. 18B shows a full sandwich including the analyte, the conjugate, and an immobilized binding partner in an embodiment of the present invention.

FIG. 18C shows a lateral flow device including the test strip of FIG. 18A, a sample collector, and a sample compressor in an embodiment of the present invention.

FIG. 19A shows another lateral flow test strip in an embodiment of the present invention.

FIG. 19B shows a full sandwich including the analyte, the conjugate, and a tagged second binding partner in an embodiment of the present invention.

FIG. 19C shows a lateral flow device including the test strip of FIG. 19A, a sample collector, and a sample compressor in an embodiment of the present invention.

FIG. 20 shows a lateral flow test strip in an embodiment of the present invention.

FIG. 21A shows yet another lateral flow test strip in an embodiment of the present invention.

FIG. 21B shows a lateral flow device including the test strip of FIG. 21A, a sample collector, and a sample compressor in another embodiment of the present invention.

FIG. 22A shows another lateral flow test strip in an embodiment of the present invention.

FIG. 22B shows a lateral flow device including the test strip of FIG. 22A, a sample collector, and a sample compressor in another embodiment of the present invention.

FIG. 23A shows a device similar to the device of FIG. 18C except that the test zone is in the sample application zone in an embodiment of the present invention.

FIG. 23B shows a device similar to the device of FIG. 19C except that the test zone is in the sample application zone in an embodiment of the present invention.

FIG. 23C shows a device similar to the device of FIG. 21B except that the test zone is in the sample application zone in an embodiment of the present invention.

FIG. 23D shows a device similar to the device of FIG. 22B except that the test zone is in the sample application zone in an embodiment of the present invention.

FIG. 24A shows a lateral flow device in an embodiment of the present invention.

FIG. 24B shows another lateral flow device in an embodiment of the present invention.

FIG. 25 shows a vertical stack in an embodiment of the present invention.

FIG. 26A shows a lateral flow device with a bather in another embodiment of the present invention.

FIG. 26B shows the lateral flow device of FIG. 26A after compression.

FIG. 27A shows a lateral flow device with a gap in another embodiment of the present invention.

FIG. 27B shows the lateral flow device of FIG. 27A after compression.

FIG. 28A shows a side view of a lateral flow device with a diverting zone and a sample compressor in an embodiment of the present invention.

FIG. 28B shows a perspective view of the lateral flow device of FIG. 28A.

FIG. 29A shows a side view of a lateral flow device with a diverting zone, a sample compressor, a sample collection device comprising a separator paper, and a chromatographic test strip in an embodiment of the present invention.

FIG. 29B shows a top down view of a section of the test strip in the embodiment of FIG. 29A.

FIG. 29C shows a top down view of a section of the test strip after the separator paper has been placed on top of the sample application zone in the embodiment of FIG. 29B.

FIG. 30 shows a lateral flow device with a diverting zone, a sample compressor, a sample collection device comprising a separator paper, and a chromatographic test strip in an embodiment of the present invention.

FIG. 31A shows a lateral flow device with a barrier in another embodiment of the present invention.

FIG. 31B shows the lateral flow device of FIG. 31A after compression.

FIG. 32A shows a lateral flow device with a gap in another embodiment of the present invention.

FIG. 32B shows the lateral flow device of FIG. 32A after compression.

FIG. 33A shows a lateral flow device with a barrier in another embodiment of the present invention.

FIG. 33B shows the lateral flow device of FIG. 33A after compression.

FIG. 34A shows a lateral flow device with a gap in another embodiment of the present invention.

FIG. 34B shows the lateral flow device of FIG. 34A after compression.

FIG. 35A shows a side view of a lateral flow device with a diverting zone and a sample compressor in an embodiment of the present invention.

FIG. 35B shows a perspective view of the lateral flow device of FIG. 35A.

FIG. 36A shows a side view of a lateral flow device with a diverting zone and a sample compressor in an embodiment of the present invention.

FIG. 36B shows a perspective view of the lateral flow device of FIG. 36A.

FIG. 37A shows a lateral flow device with a barrier in another embodiment of the present invention.

FIG. 37B shows the lateral flow device of FIG. 37A after compression.

FIG. 38A shows a lateral flow device with a gap in another embodiment of the present invention.

FIG. 38B shows the lateral flow device of FIG. 38A after compression.

FIG. 39A shows a side view of a lateral flow device with a diverting zone and a sample compressor in an embodiment of the present invention.

FIG. 39B shows a perspective view of the lateral flow device of FIG. 39A.

FIG. 40A shows a side view of a lateral flow device with a diverting zone, a sample compressor, a sample collection device comprising a separator paper, and a chromatographic test strip in an embodiment of the present invention.

FIG. 40B shows a top down view of a section of the test strip in the embodiment of FIG. 40A.

FIG. 40C shows a top down view of a section of the test strip after the separator paper has been placed on top of the sample application zone in the embodiment of FIG. 40B.

FIG. 41 shows a lateral flow device with a diverting zone, a sample compressor, a sample collection device comprising a separator paper, and a chromatographic test strip in an embodiment of the present invention.

FIG. 42A shows a lateral flow device with a bather in another embodiment of the present invention.

FIG. 42B shows the lateral flow device of FIG. 42A after compression.

FIG. 43A shows a lateral flow device with a gap in another embodiment of the present invention.

FIG. 43B shows the lateral flow device of FIG. 43A after compression.

FIG. 44A shows a lateral flow device with a bather in another embodiment of the present invention.

FIG. 44B shows the lateral flow device of FIG. 44A after compression.

FIG. 45A shows a lateral flow device with a gap in another embodiment of the present invention.

FIG. 45B shows the lateral flow device of FIG. 45A after compression.

FIG. 46A shows a side view of a lateral flow device with a diverting zone and a sample compressor in an embodiment of the present invention.

FIG. 46B shows a perspective view of the lateral flow device of FIG. 46A.

FIG. 47A shows a side view of a lateral flow device with a diverting zone and a sample compressor in an embodiment of the present invention.

FIG. 47B shows a perspective view of the lateral flow device of FIG. 47A.

FIG. 48 shows a preferred configuration for a sample analysis device in another embodiment of the present invention.

FIG. 49 shows another embodiment of a direct correlation inhibition assay with a hapten zone.

FIG. 50 shows an embodiment of a test strip using Fclock.

FIG. 51 shows a sample analysis device with C18 at the test line in an embodiment of the present invention.

FIG. 52 shows another embodiment of a sample analysis device with C18 at the test line.

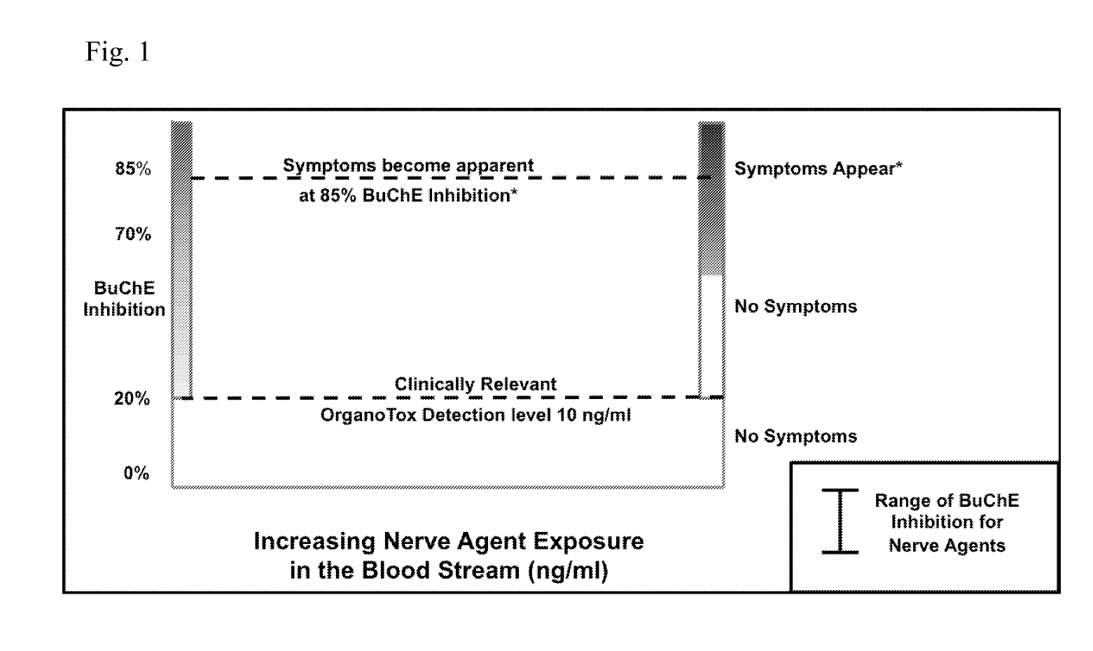

### DETAILED DESCRIPTION OF THE INVENTION

A rapid, point-of-care assay detects clinically relevant organophosphate (OP) poisoning after low-level exposure to Sarin, Soman, Tabun, or VX chemical nerve agents. The test utilizes either a finger stick peripheral blood sample or a plasma specimen. The blood sample or plasma specimen is taken by direct sampling.

While high-level nerve agent exposure can quickly lead to death, low-level exposure produces vague, nondescript signs and symptoms that are not easily clinically differentiated from other conditions. A 10-minute point-of-care assay detects blood protein adducts resulting from low level exposure to organophosphate nerve agents.

After inhalation or contact with skin, nerve agents enter the blood and immediately combine with cholinesterases and other blood proteins including albumin, the most abundant serum protein, to form blood protein-nerve agent adducts. Nerve agents are unstable in pure aqueous solutions and are rapidly degraded upon entering the body. Therefore, analysis for the presence of intact agents in blood is inappropriate unless samples are collected immediately after exposure. However, Sarin, Soman, and Tabun phosphylate a tyrosine residue on albumin in human blood. The blood protein adducts are immediately detectable, do not age rapidly, are extremely stable, persist in the blood stream for up to 20 days post exposure, and are not degraded by therapy with oximes. These protein nerve agent adducts offer a stable and reliable alternative for confirming low level nerve agent poisoning.

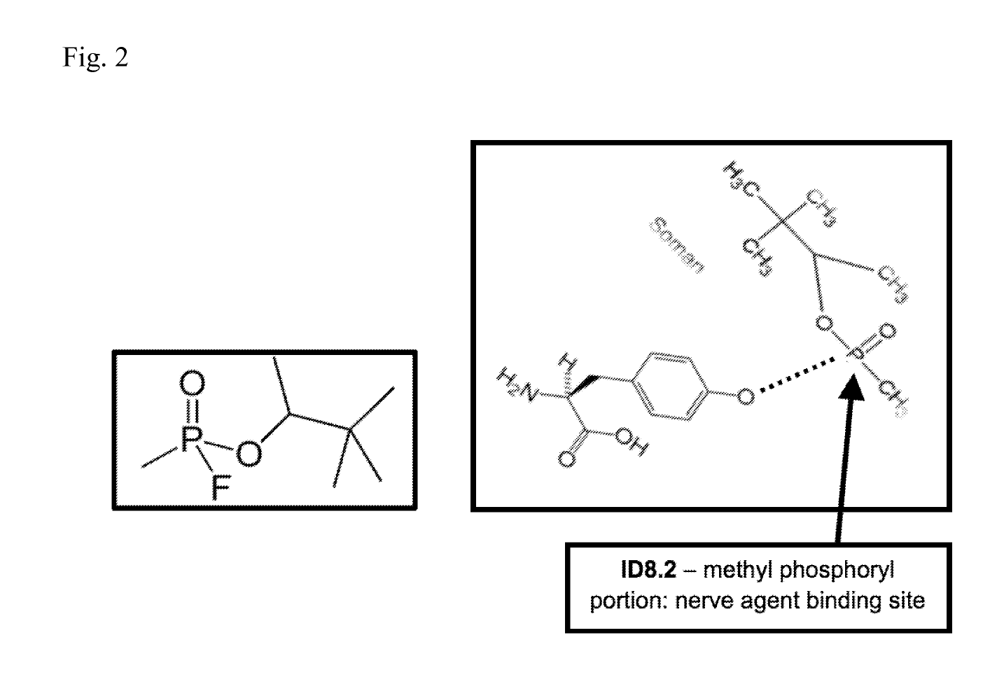

Organophosphate nerve agents phosphylate the tyrosine residues on albumin Tyrosine is one of the naturally occurring amino acids that binds with phosphate groups. There are other tyrosine residues on albumin and other blood proteins that can adduct with nerve agents, however, tyrosine 411 residue on albumin has been shown to be the most reactive to form a covalent bond with the benzene ring oxygen leading to the blood protein adduct shown in FIG. 2.

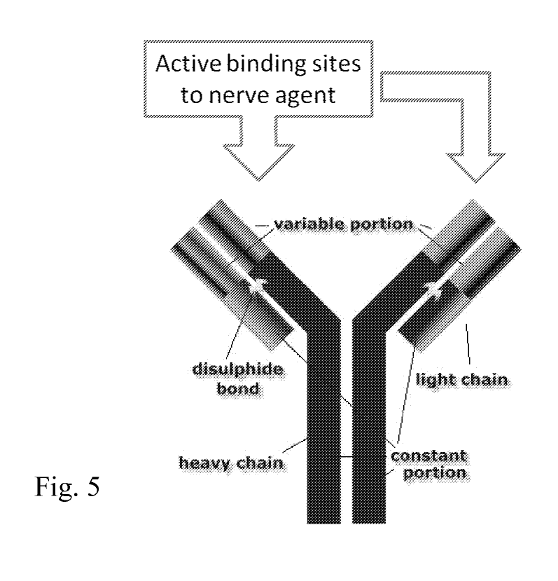

ID8.2, shown in FIG. 5, is a highly specific antibody developed to detect the presence of blood protein nerve agent adducts (“Role of immunogen design in induction of Soman-specific monoclonal antibodies”, Johnson J K, Cerasoli D M, Lenz D E. U.S. Army Medical Research Institute of Chemical Defense. Immunology Letters (2005) 96:121-127, herein incorporated by reference). ID8.2 binds to Soman (GD), Sarin (GB), Tabun (GA), and VX.

There is a common architecture around the phosphorus atom in all chemical nerve agents. The ID8.2 antibody binds at the methyl phosphoral portion of the albumin tyrosine 411 nerve agent adduct. While similar structurally to nerve agents, pesticides and pesticide metabolites lack a chiral center and do not bind to the albumin tyrosine to form a blood protein adduct that is recognized by the ID8.2 antibody.

An exposure leading to blood nerve agent levels of 10 ng/ml significantly inhibits BuChE, and neurological function becomes compromised, requiring immediate medical care and intervention. Therefore, the clinically relevant lower limit of detection of a qualitative, optically interpreted assay should be at a chemical nerve agent blood exposure level of 10 ng/ml. If an electronic reader is used to quantitate the results, the lower limit of detection should extend below the clinically relevant levels in order to provide a broader range of values for diagnostic interpretation. In preferred embodiments, the methods and devices described herein detect nerve agent blood exposure at levels less than or equal to 10 ng/ml. In one preferred embodiment, the methods and devices detect nerve agent blood exposures at levels in the range of 1 ng/ml through 10 ng/ml.

At 10% to 20% plasma BuChE inhibition, albumin tyrosine adducts are present that represent nerve agent exposure consistent with Sarin at 2 ng/ml, Soman at 1 ng/ml, and Tabun at 5 ng/ml. Additionally, at Sarin exposure of 10 ng/ml, it has been reported that tyrosine adducts develop and represent 70% or greater BuChE inhibition. Since albumin tyrosine adducts are present that represent nerve agent exposure at these levels, in preferred embodiments, the devices and methods are able to detect these extremely low levels of these particular nerve agents. Using a non-invasive finger-stick blood sample in combination with a lateral flow assay provides test results in 10-15 minutes.

Whole blood/plasma is preferably used as the best sample matrix. This permits rapid screening of a large at-risk population at the event scene. The nerve agents are imediately detectable within a few minutes after exposure, and have a long lasting detection window (days). Adducts form with all blood proteins. Obtaining a blood sample is non-invasive. While other matrices could be used, they are not as effective. For example, urine has a time lag for detectable markers to appear. Metabolites and hydrolysis products are only present in body fluids in very low concentrations after exposure. Collection of a cerebrospinal fluid (CSF) sample is invasive and not point-of-care.

The methods and devices described herein protect first responders and at-risk personnel. Routine testing of at-risk personnel leads to early identification and treatment in the event of low-level or chronic exposure. The methods and devices also improve health outcomes at the event scene. They avoid high risk resuscitating drugs and anesthetics that can be deadly for those who have been exposed to chemical nerve agents. Screening with these methods and devices ensures safety and treatment outcomes. This preferably includes screening of the “worried” or “walking well” to confirm if exposure has occurred. Exposure screening provides the most appropriate and expedient care, conserving limited therapeutic treatments for those in need. The assays expedite treatment and improve outcomes with immediate, on-site testing and treatment.

In some preferred embodiments, the devices described herein are packaged with companion antidotes and/or therapeutic drugs.

Exposure occurs when a chemical nerve agent enters the blood stream via inhalation or absorption through the skin. In the blood stream, the nerve agent immediately begins adducting to all blood proteins including Butyrylcholinesterase, albumin, and immunoglobulins. Nerve agents enter red and white blood cells and cross the blood brain barrier to adduct to acetycholinesterase. Nerve agent specific monoclonal antibodies detect the blood protein adducts resulting from nerve agent exposure.

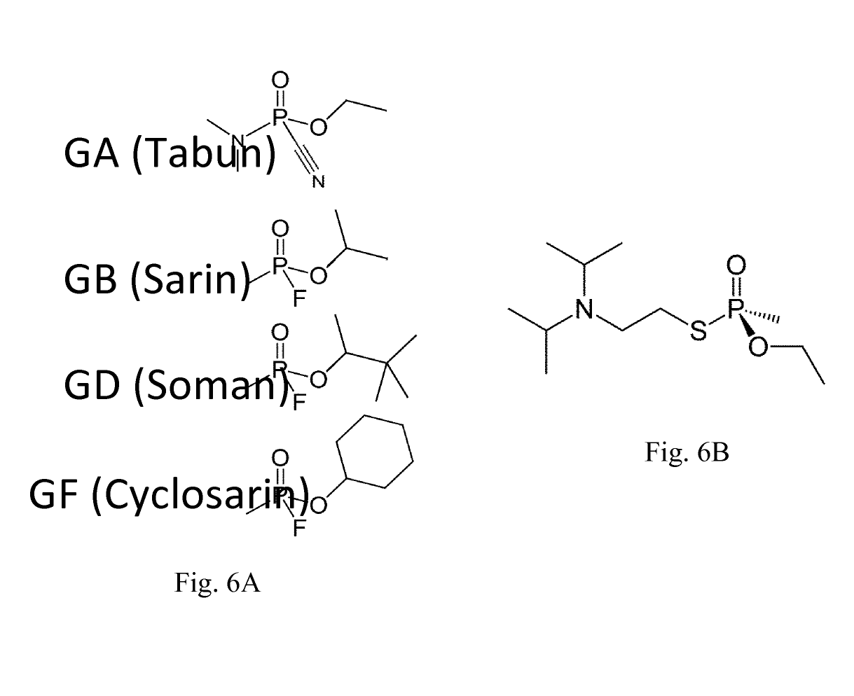

Partial chemical structures of the nerve agent adducts to albumin are shown in FIG. 6A. Note the common architecture around the phosphorus atom, shown in FIG. 6B. This is the epitope that IDB recognizes in Phosphinates. OP pesticides are typically phosphates without a chiral center at the phosphorus atom.

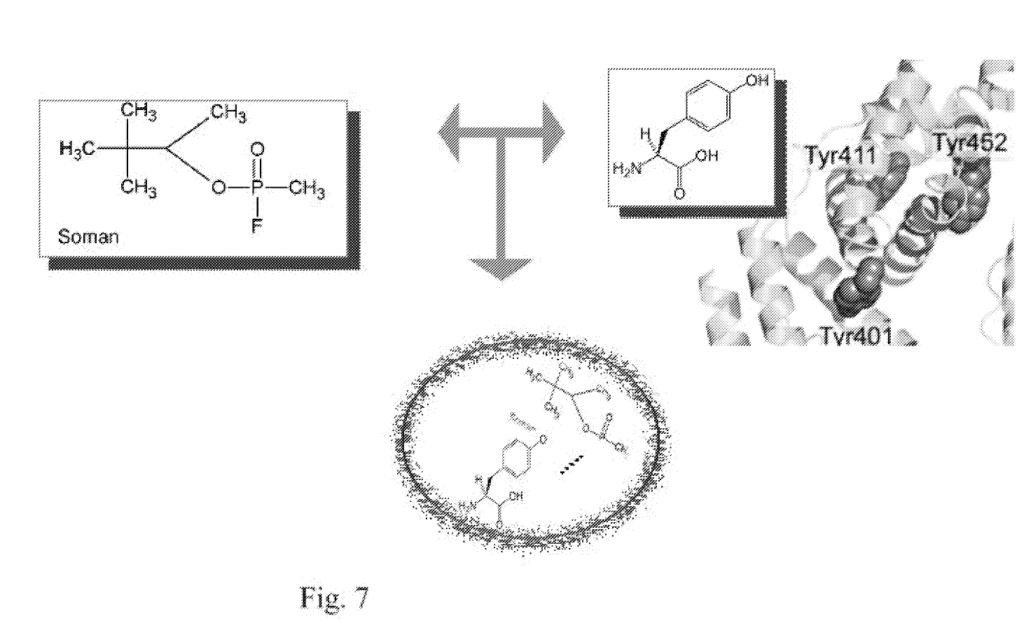

Tyrosine, one of 20 natural amino acids, binds with phosphate groups. Tyrosine 411 of human albumin is an established site for covalent attachment of many OPs including Soman, Sarin, Tabun, and VX. Tyrosine 411 is the most active site against nerve agents. Many other less active Tyrosine and Serine sites are present in Albumin Adduct formation for Soman, as an example, occurs as shown in FIG. 7. Soman loses Fluorine and Tyrosine 411 loses Hydrogen. Then, the Soman Phosphate Group and Tyr 411 Benzene ring Oxygen form the covalent bond.

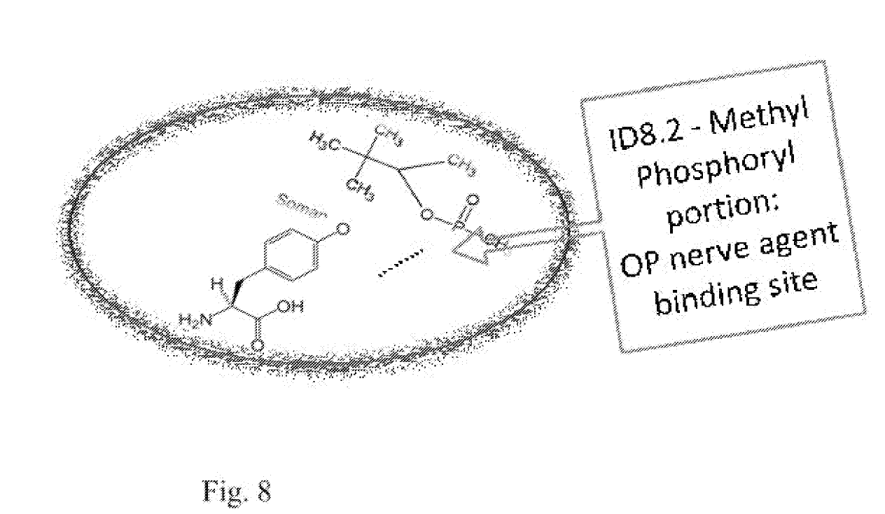

The nerve agent binding site for antibody ID8.2 (Johnson, J. M., Cerasoli, D. M. and Lenz, D. E., “Role of Immunogen Design in Induction of Soman-Specific Monoclonal Antibodies”, (2005) Immunology Letters 96, 121-127, herein incorporated by reference) is shown in FIG. 8.

In initial testing, one of the assays described herein was used to detect the presence of blood protein-nerve agent adducts in exposed blood samples. In order to mimic the in vivo exposure as closely as possible, nerve agents stored in organic solvents were spiked in minute quantities into whole blood samples. For performance testing, 40 plasma samples were spiked with Sarin, Soman, Tabun, or VX and 10 normal plasma samples were used as the negative control. The 40 nerve agent-spiked plasma samples included 10 replicates of each agent. At the clinically relevant low-level exposure of 10 ng/ml, the test demonstrated 100% sensitivity for Soman, Tabun, and VX and 80% sensitivity for Sarin. The test demonstrated greater than 97% specificity with 150 blood samples obtained from healthy adults. No cross-reactivity or interference from pesticide precursor compounds was found.

A rapid test for nerve agent exposure helps identify affected patients earlier in the clinical course and triggers more appropriate medical management in a more timely manner.

### a Direct Sandwich Assay for CWNA-Albumin Adducts

Soman-Albumin adducts formed due to exposure to Soman are found in plasma/serum. The hapten Soman may be accessible to bind with the Monoclonals BE2 and/or ID8.2 conjugated to a visual tag, preferably spiked with fluorescenated latex bead conjugates in some embodiments. The sandwich forms with Albegone binding to the Albumin portion of the adduct and the monoclonal antibodies binding to the haptenic Soman in the adduct.

In a direct sandwich assay for chemical warfare nerve agents, Albumin adducts are preferably captured by the recombinant Albegone at the test line and the adducted chemical warfare nerve agents are preferably detected with their appropriate monoclonal antibodies on suitable visual or fluorescence conjugates.

Albegone is a recombinant protein that binds specifically with mammalian Albumin. In preferred embodiments, Albegone is immobilized onto the nitrocellulose membrane as the test line. In other preferred embodiments, monoclonal or polyclonal antibodies to mammalian albumin could alternatively be used as the test line.

As discussed above, Soman, an organophosphate chemical warfare nerve agent, forms a relatively stable adduct with human Serum Albumin by binding to the Tyrosyl residue on Human Serum Albumin Monoclonal antibodies BE2 and ID8.2 were raised against Soman chemically conjugated to Human or Bovine Serum Albumin. These monoclonal antibodies recognize the pinacolyl moiety of Soman. While Soman is being used in this example, the test could alternatively or additionally test for other chemical warfare nerve agents which bind to the Tyrosyl residue on Human Serum Albumin, including, but not limited to, Sarin, Tabun, VX, or Cyclosarin.

In this assay format, the commonly used Bovine Serum Albumin, a common blocking agent, is replaced, for example with casein, gelatin gelatin or any non-albumin related proteins in the assay.

The monoclonal antibodies are conjugated to colloidal gold or dyed latex beads and act as the conjugate (the first binding partner) in the assay. The rest of the surface of the colloidal particle is blocked with casein or gelatin. The recombinant Albegone is preferably immobilized on the nitrocellulose membrane as the test line.

Serum or plasma containing the Soman-albumin adduct from animals or humans exposed to chemical warfare nerve agents is added as the sample. The Albumin portion of the chemical warfare nerve agent adduct is captured by the recombinant Albegone at the test line. In one preferred embodiment, the monoclonal antibody on colloidal gold or latex beads then binds to the chemical warfare nerve agent portion of the protein adduct, giving rise to a visible discernible test line. In other embodiments, any of the configurations discussed above, for example configurations where ½ of the analyte complex sandwich is formed before the sample reaches the test line, could alternatively be used.

In a preferred embodiment, the assay is a lateral flow immunoassay incorporating a lateral flow chromatographic test strip. Samples, preferably blood, are added directly to the test strip. In some preferred embodiments, a sample compressor is added to the device after the sample has been collected and placed on the test strip. The sample compressor does not need to be sterile. In some preferred embodiments with the sample compressor, the sample compressor does not include any reagents for the assay.

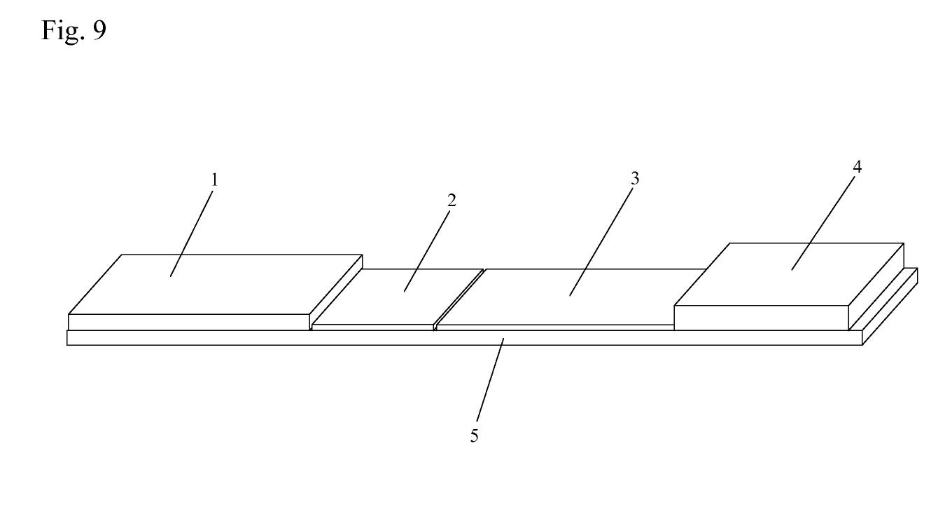

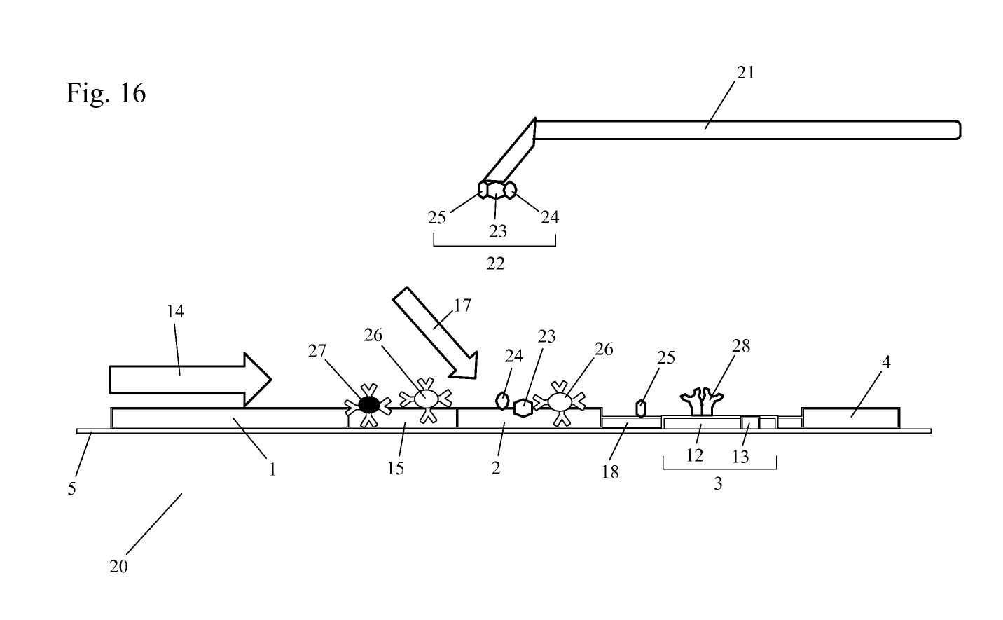

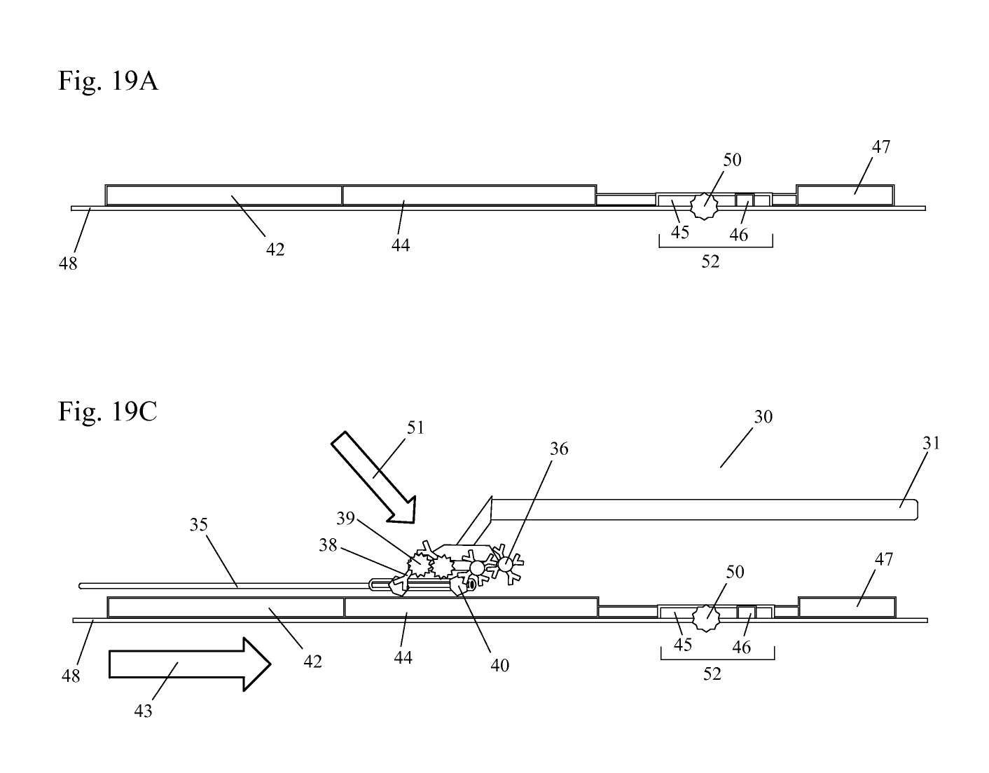

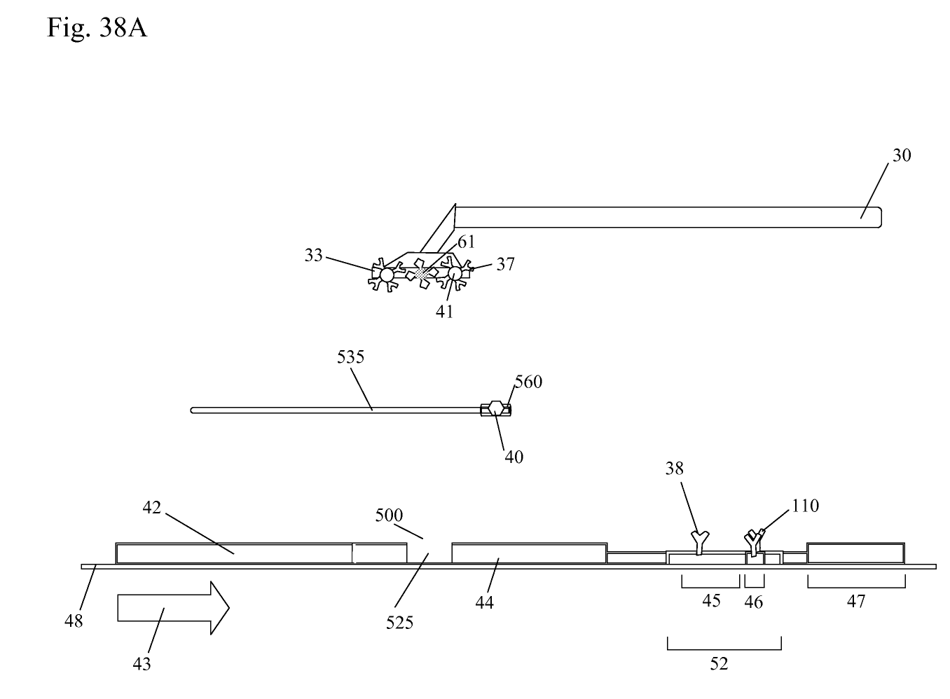

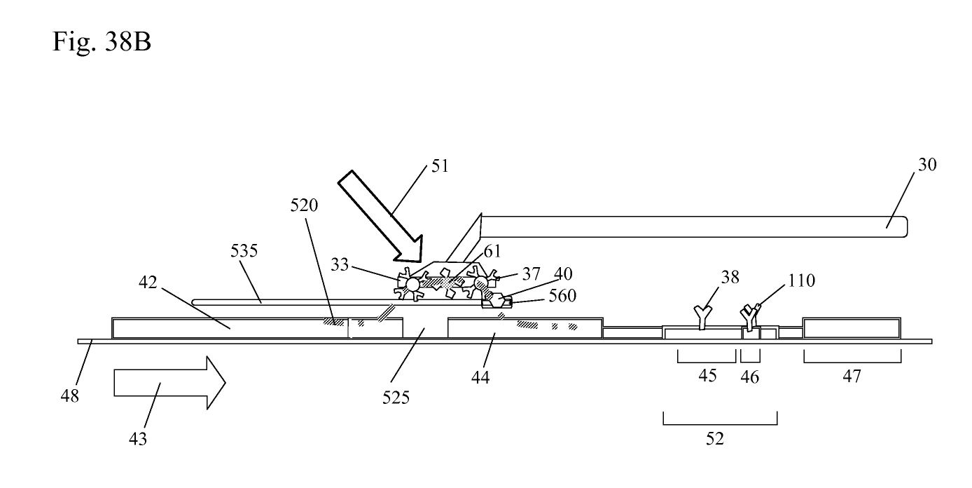

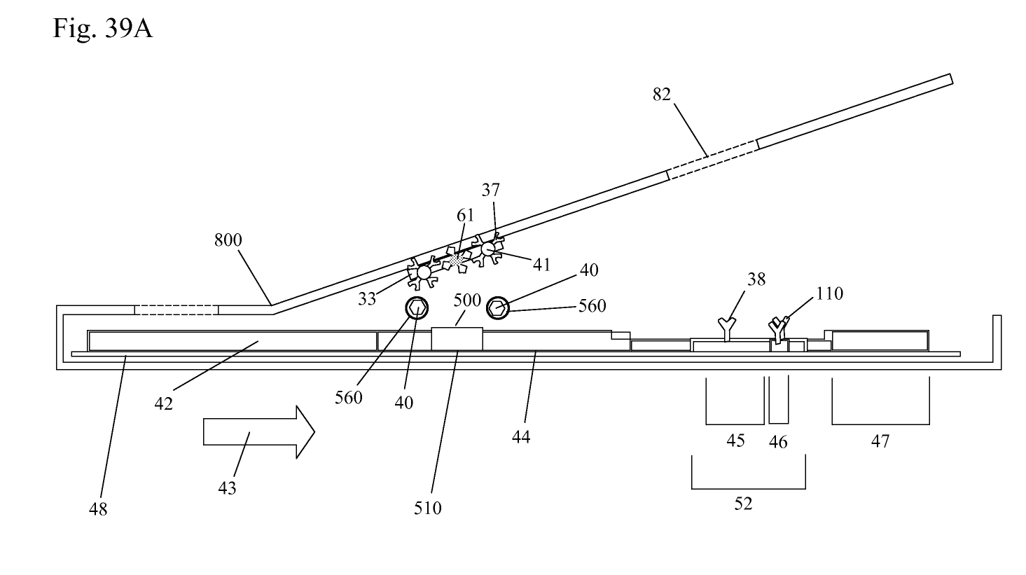

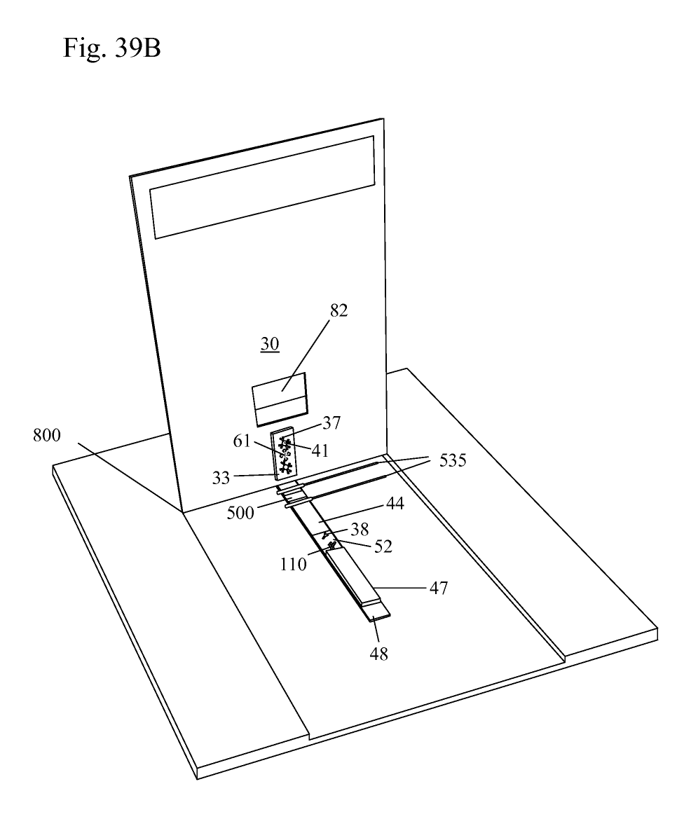

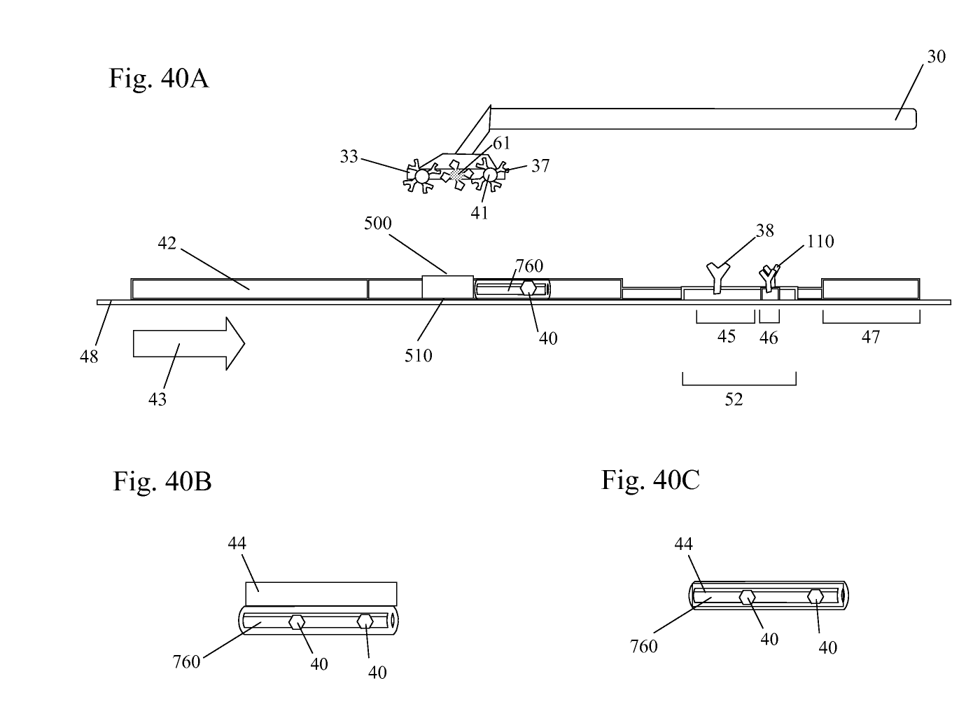

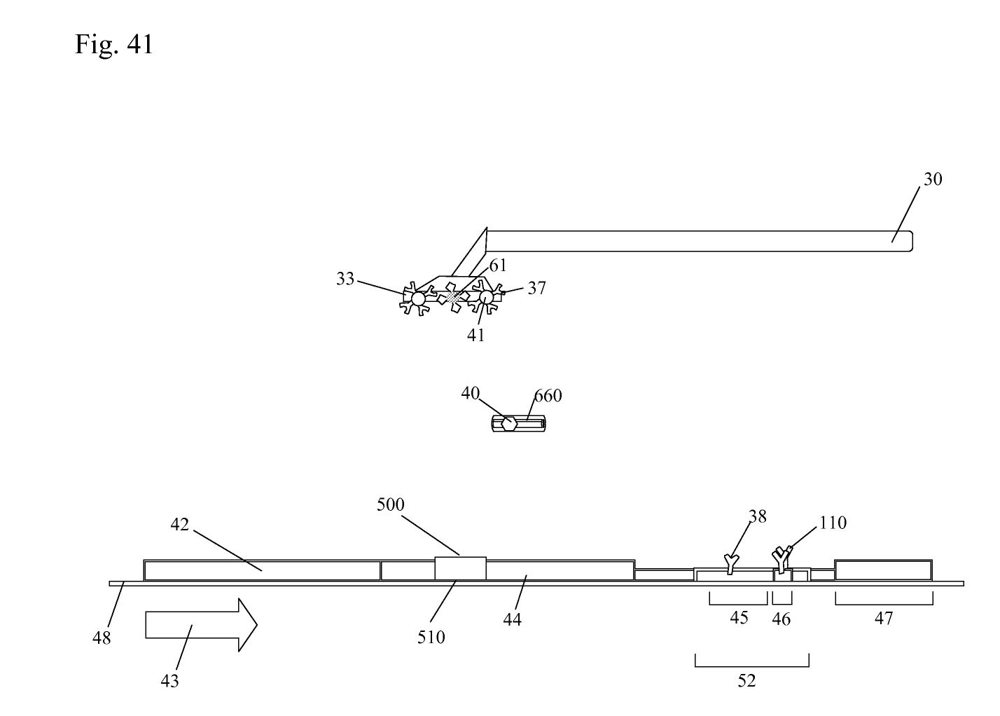

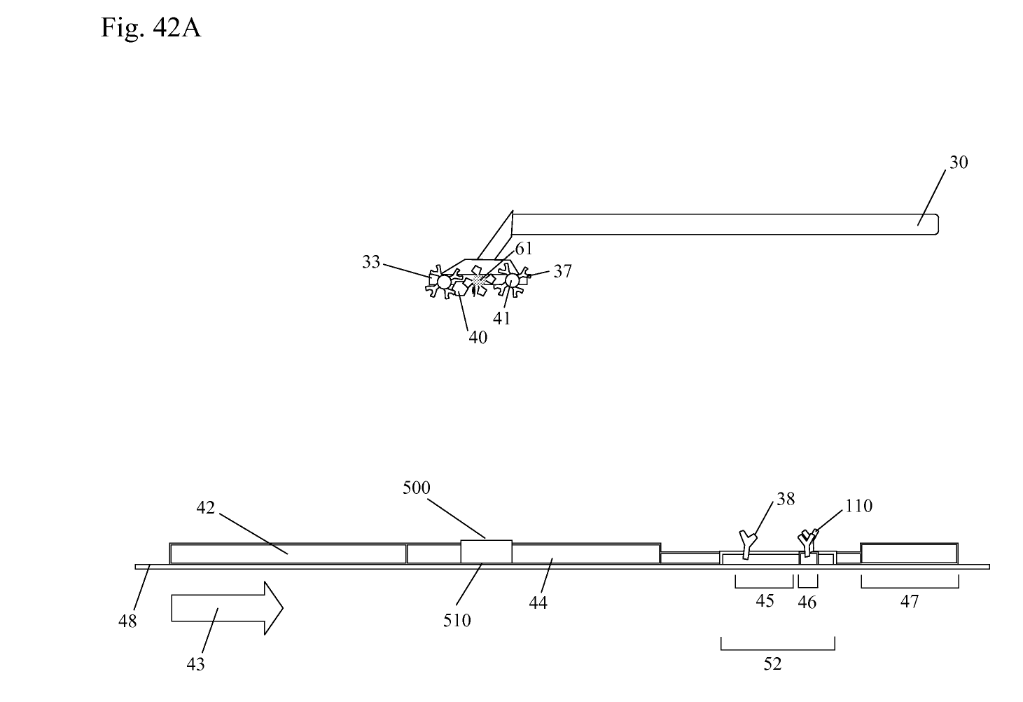

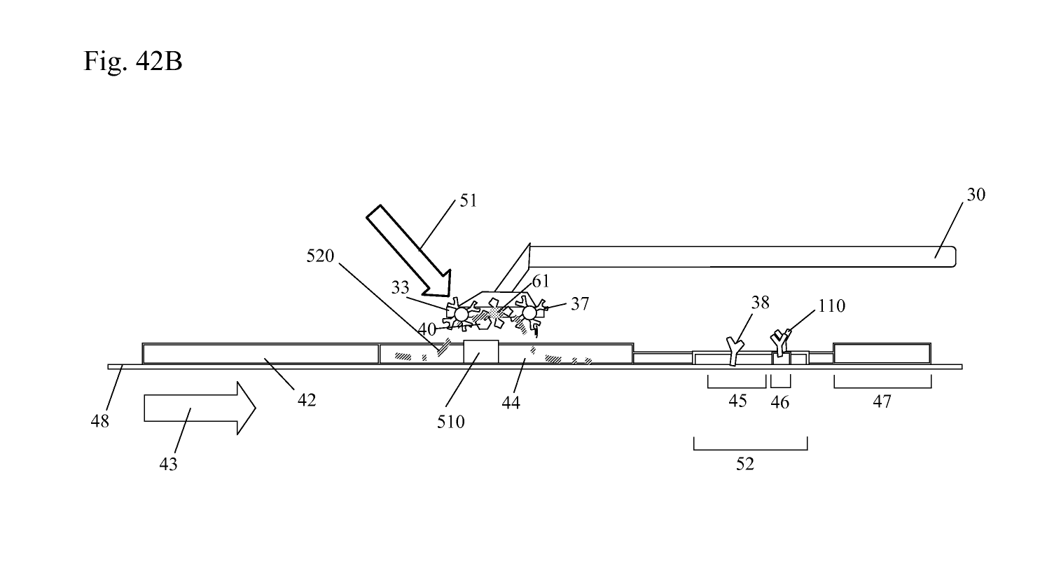

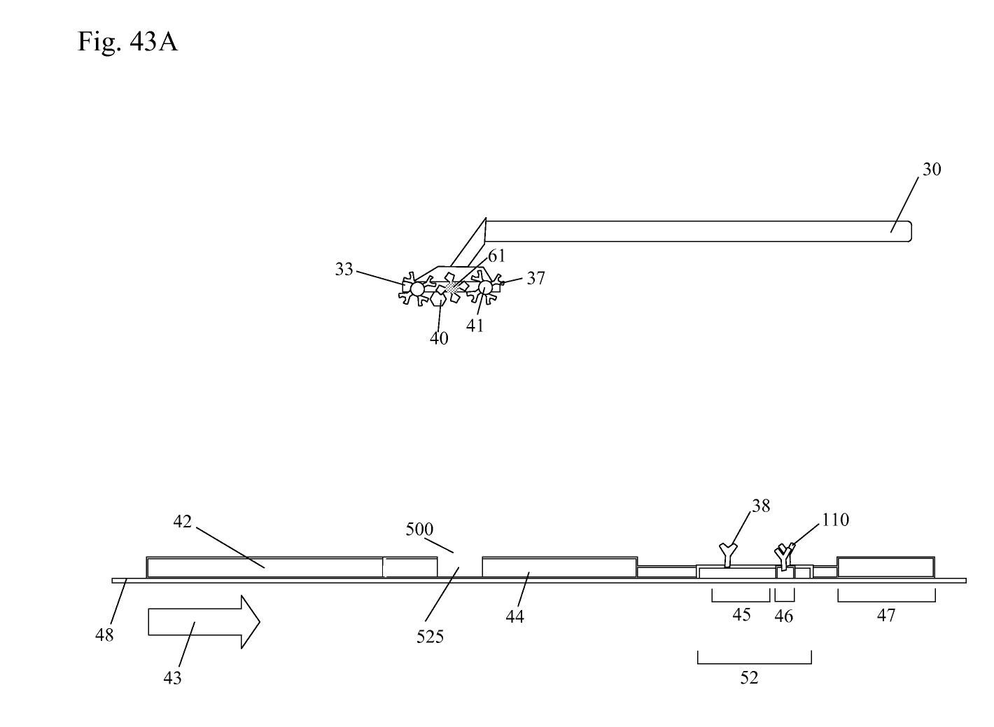

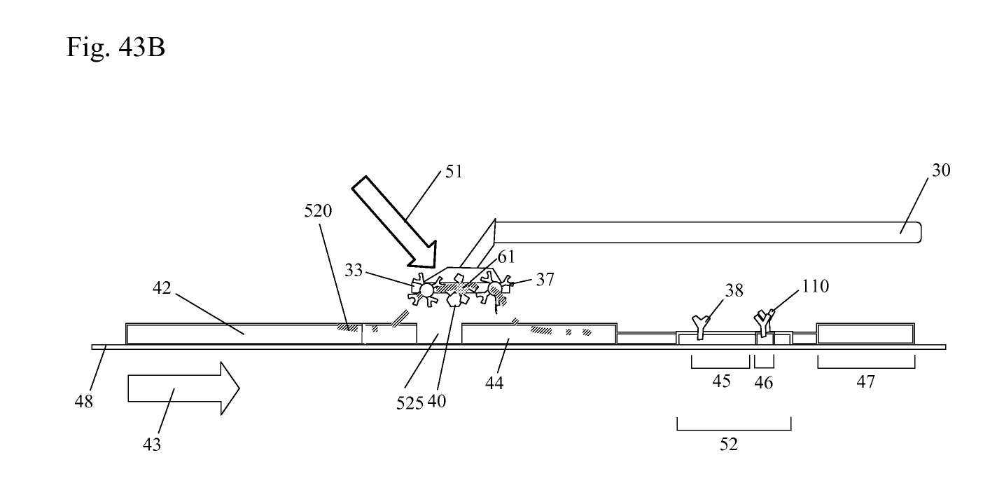

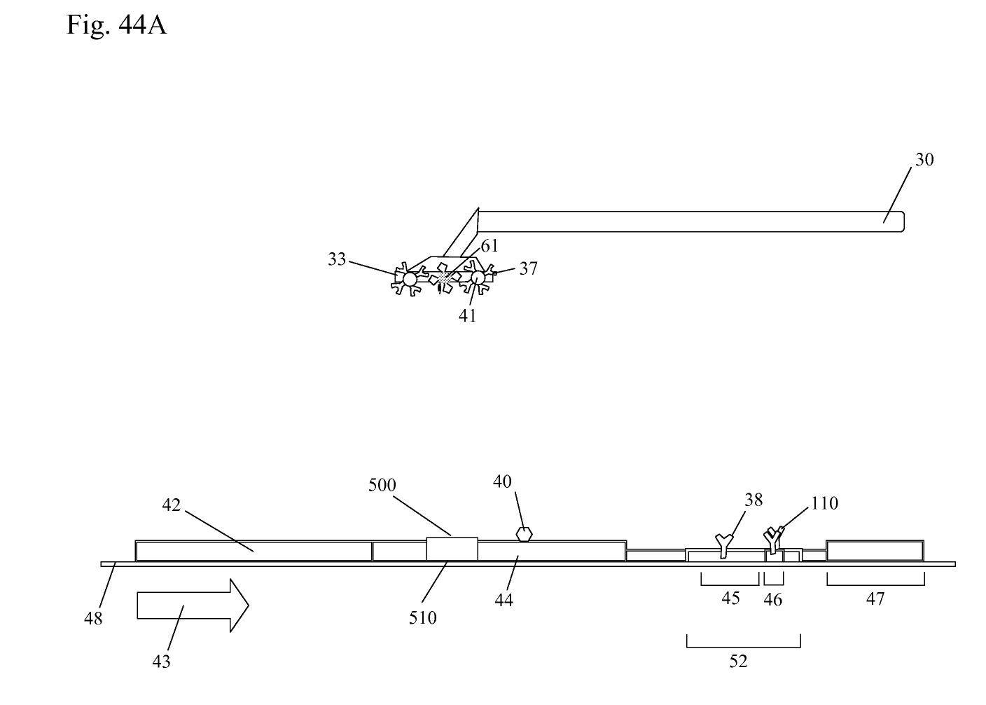

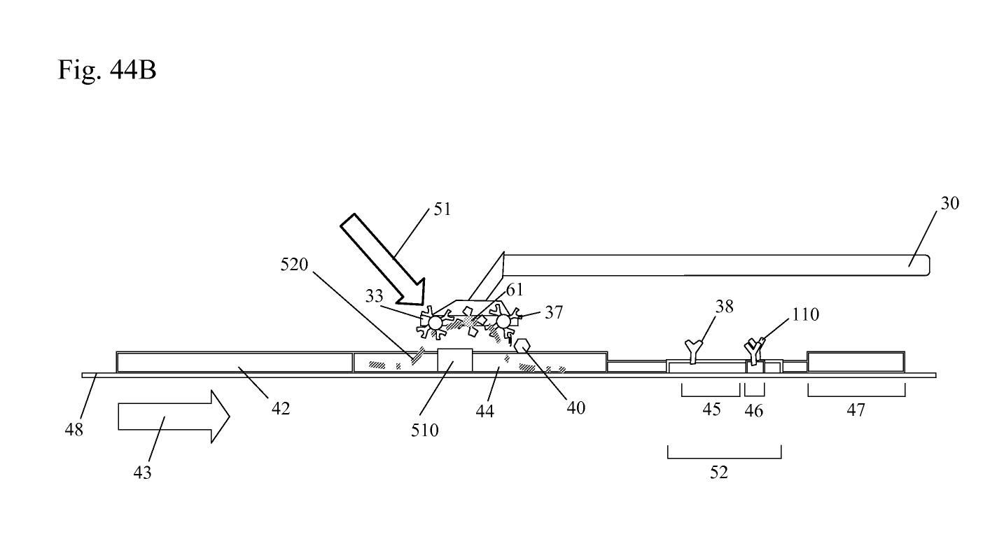

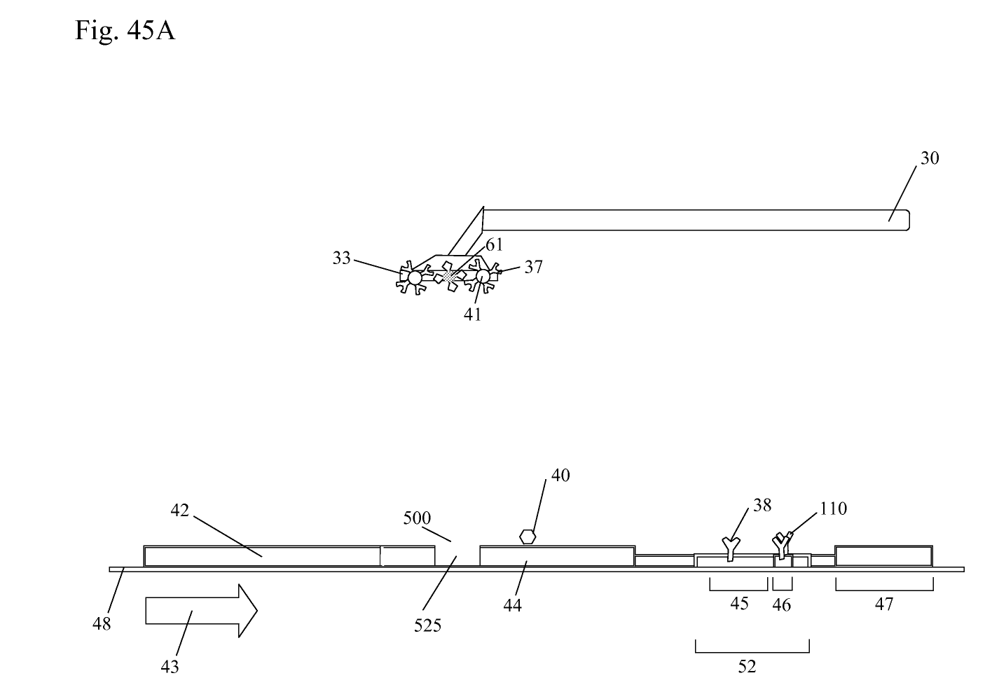

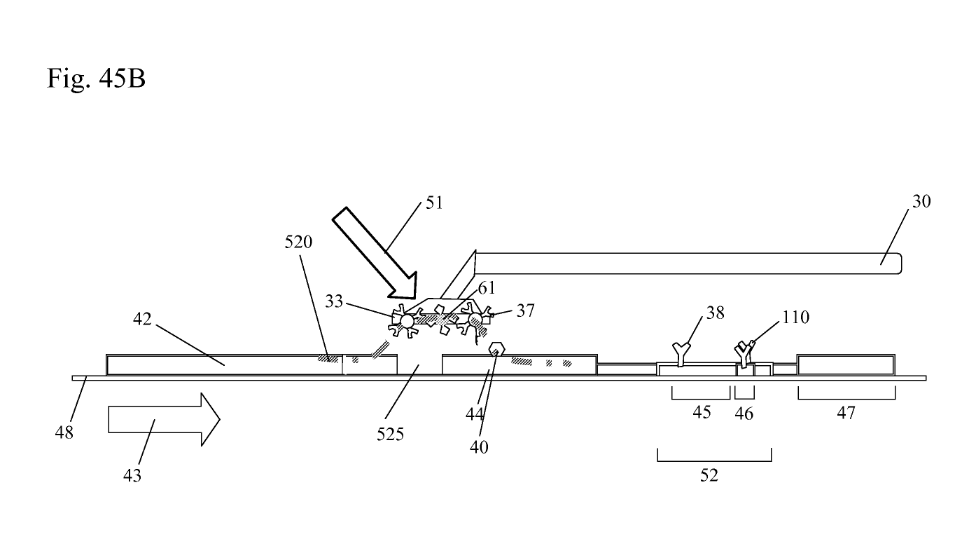

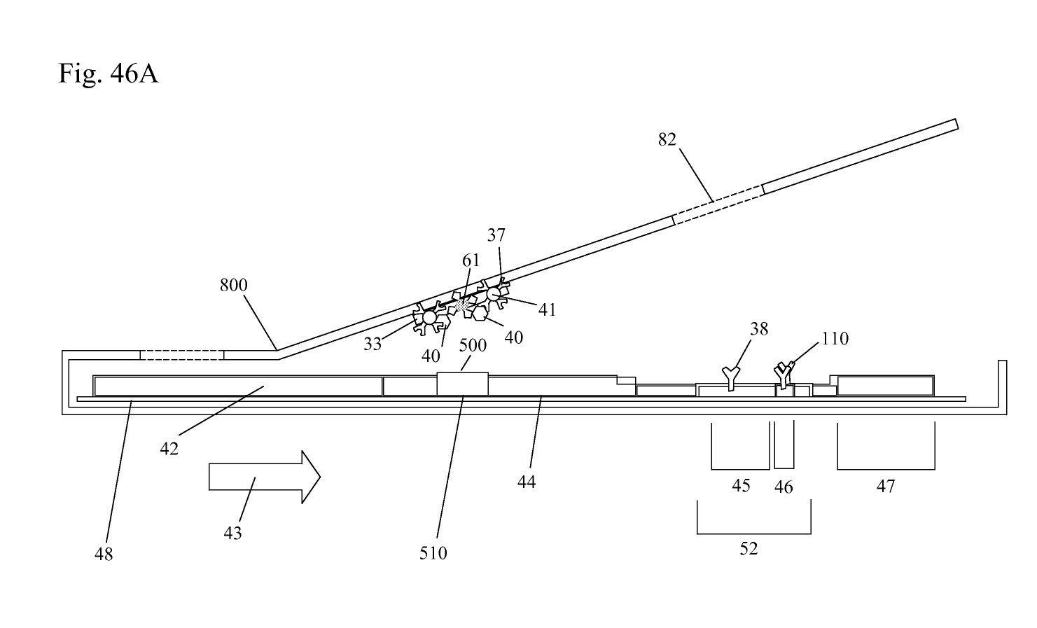

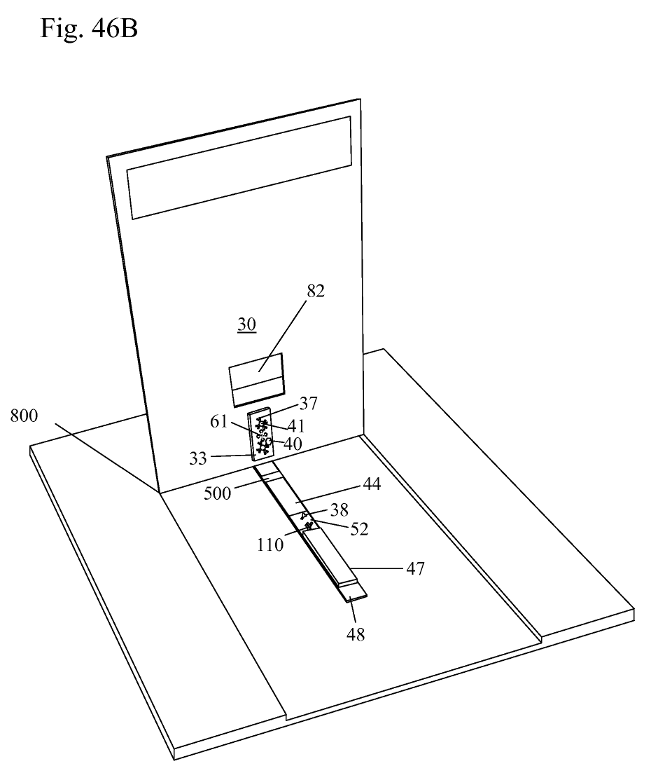

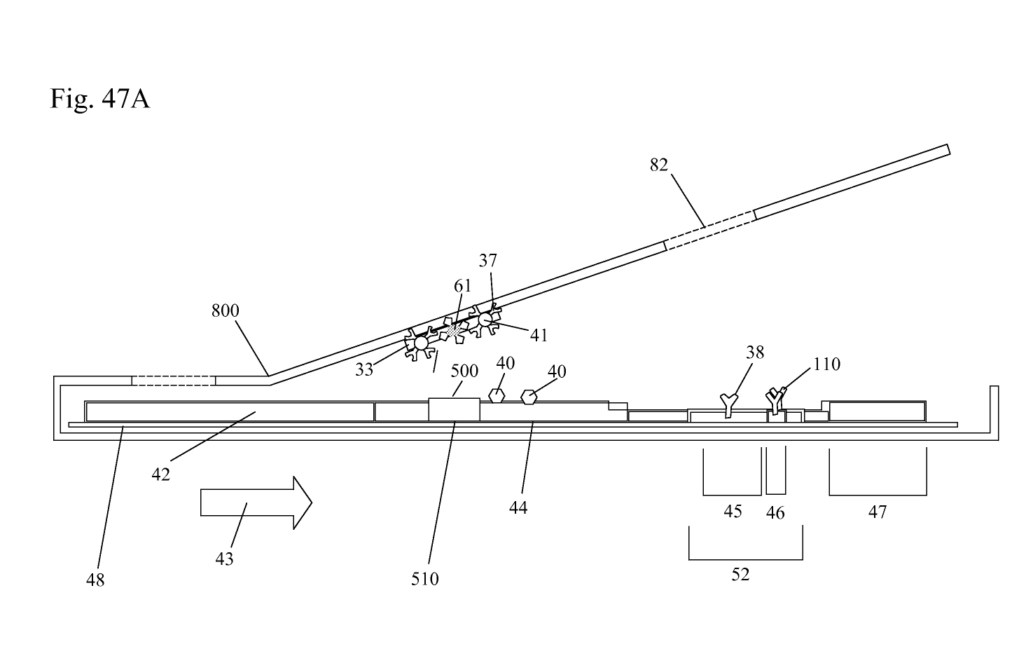

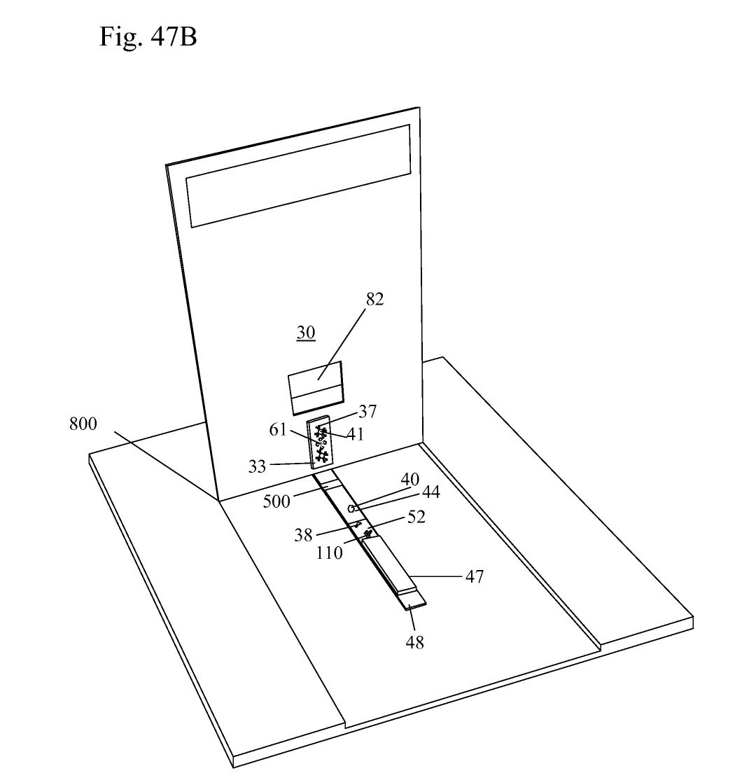

The direct sandwich assay may employ any of the assay configurations described herein in FIGS. 9-47, or any other assay configurations known in the art. The BE2 and ID8.2 antibodies conjugated to a label and albumin are preferably placed in the conjugate zone (on the test strip or on the sample compressor, depending upon the embodiment) and/or in the sample application zone. Albegone or anti-mammalian albumin (preferably anti-human albumin) is placed at the test line (for example in the assay configurations shown in FIGS. 16, 18, 23A, and 37-47) or, in embodiments where the full sandwich is formed before the sample reaches the test line and tags, such as biotin and avidin, are used (see, for example in the assay configurations shown in FIGS. 19-22, 23B-23D, and 26-36), Albegone acts as the mobile second binding partner for the chemical warfare nerve agent.

The visual test can be made more sensitive by incorporating fluoresceinated beads. Any of the assays and methods of US Patent Publication No. 2009/0289201, published Nov. 26, 2009, entitled “COMBINED VISUAL/FLUORESCENCE ANALYTE DETECTION TEST”, incorporated herein by reference, may be used to enhance the sensitivity of the test.

The devices and methods may be used to test animals in the field. These animals would have the chemical warfare nerve agents in their blood and adducting to blood proteins where the chemical warfare nerve agents are present. By testing the animal blood for the presence of the chemical warfare nerve agent (CWNA)-albumin adduct, this serves as a “Bio-screen” or “Bio-monitoring” for these agents. The exposure to the chemical warfare nerve agents by humans may also be assessed by this test.

### a Positive Correlation Point of Care Immunoassay to Detect the Chemical Warfare Nerve Agents

As discussed above, the monoclonal antibody ID8. 2 binds to Soman, Sarin, VX and Tabun. The monoclonal antibody BE2 binds to Soman and some of its analogs. In preferred embodiments, different colored latex beads are combined as the visual tag. In some embodiments, fluorescent latex beads are added to improve the sensitivity.

A competitive inhibition test is established either with a hapten zone or a blocking antibody zone.

Similar to the embodiment described above, the nitrocellulose membrane preferably has Albegone immobilized at the test line. In other preferred embodiments, monoclonal or polyclonal antibodies to bovine serum albumin or human serum albumin are immobilized at the test line.

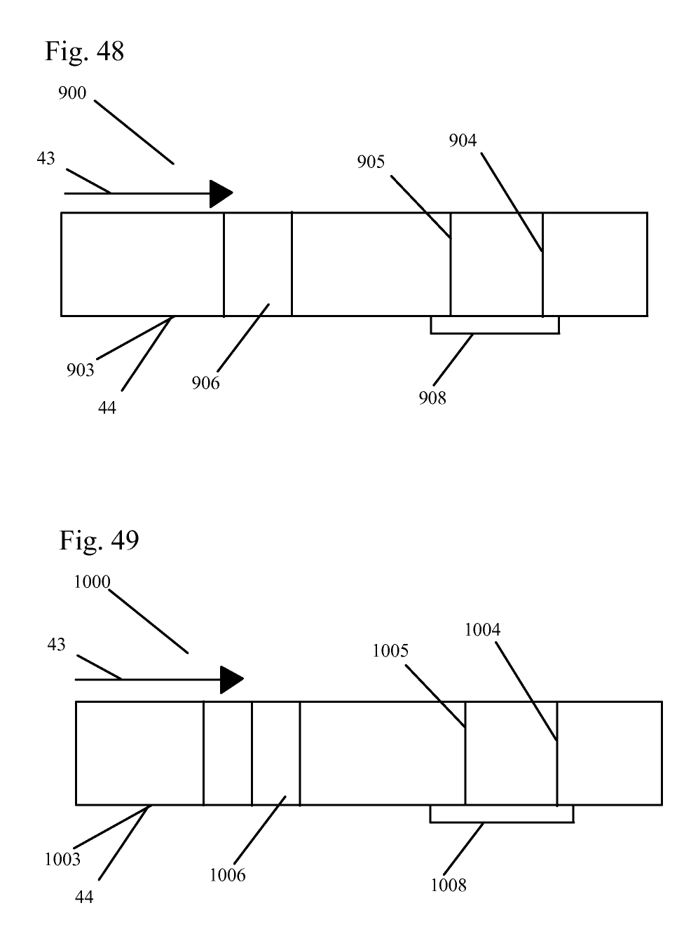

FIG. 48 shows one preferred configuration for a sample analysis device 900 in this embodiment, with an antibody blocking zone/hapten zone 906.

In one general form of this test, a lateral flow immunoassay includes at least a sample application zone 44, a conjugate zone 903 (which may overlap the sample application zone), a blocking antibody zone 906 and a test line 905. The test line 905 preferably has Albegone immobilized on it to capture any albumin that comes into the detection zone 908. A chemical warfare nerve agent coupled (for example, chemically coupled either through carboxyl or amino ends) to human serum albumin or bovine serum albumin, which provides a binding site for the antibodies in the blocking antibody zone, is conjugated to a label and preferably included in the conjugate zone 903. In between the sample application zone/conjugate zone 44/903 (preferably close to the sample application zone) and the test line 905 is a blocking antibody zone 906. The blocking antibody zone 906 includes antibodies immobilized in the blocking antibody zone 906. These antibodies bind both to the binding site on the conjugated albumin and the target analyte. The target analyte in the sample and the conjugated albumin compete for the binding sites of the antibodies immobilized in the blocking antibody zone 906.

If a sample without the target is added to the assay, the antibodies in the immobilized blocking antibody zone 906 bind to the antibody binding site on the conjugated albumin So, all of the labeled conjugate binds to the antibodies in the blocking antibody zone 906. Consequently, no detectable label makes it to the test line 905.

If a sample with the target is added to the assay, the chemical warfare nerve agents compete with the conjugated albumin for the binding sites on the antibodies in the blocking antibody zone 906. So, some of the conjugated albumin is able to travel to the test line 905. Albegone binds to the albumin, creating a detectable label and a positive result at the test line 905.

In another preferred embodiment, the chemical warfare nerve agent is adducted to human or bovine albumin and the adducted albumin is purified and conjugated to a suitable label and used as the conjugate in the embodiment described above.

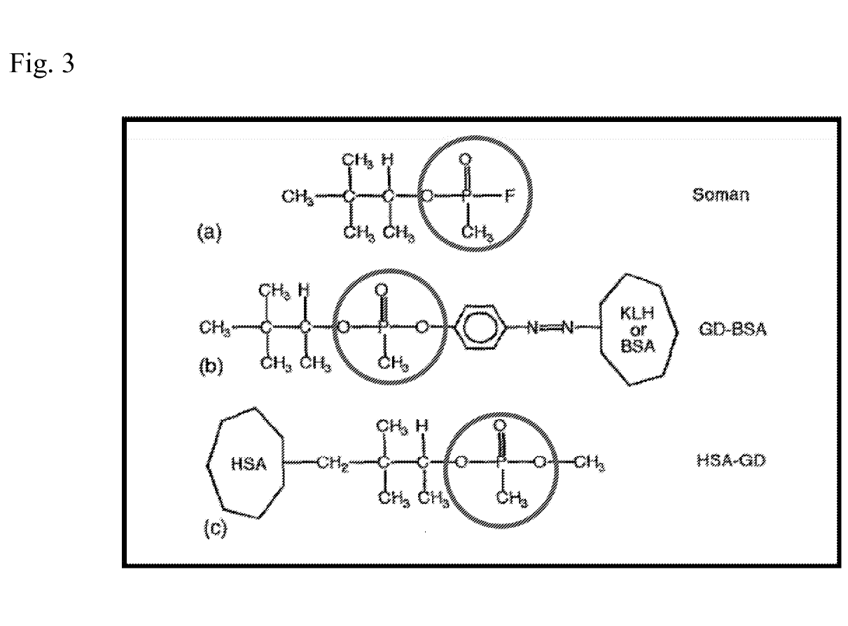

In one preferred embodiment, the blood sample is added to the test device 900 at the sample application zone 44, or pad. The conjugate zone 903 is preferably in the same location as the sample application zone 44, and preferably includes a mixture of blue latex beads conjugated to GD-bovine serum albumin and red latex beads conjugated to human serum albumin-GD. In some preferred embodiments, the conjugate zone 906 also has colorless red flash fluorescent beads conjugated to GD-bovine serum albumin (GD-BSA, see FIG. 3B) or human serum albumin-GD (HSA-GD, see FIG. 3C), either separately or together. Preferably close to this sample application zone 44, the nitrocellulose includes an antibody zone 906, where both BE2 and ID8.2 monoclonal antibodies are immobilized. GD-BSA and HSA-GD are both Soman conjugates, which are used as immunogens.

Gelatin, casein or other non-albumin proteins replace bovine serum albumin in the running buffer, and any other buffers, including, but not limited to, conjugate and sample pad release buffer, as well as the blocking protein in latex bead conjugation. The only bovine serum albumin and human serum albumin that binds to Albegone is from GD-bovine serum albumin or human serum albumin-GD. In some preferred embodiments, rabbit anti-chicken is used at the control line 904.

When chemical warfare agents are absent, all the latex bead conjugates bind to the antibody zone 906 where both BE2 and ID8.2 are immobilized, because HSA-GD has binding sites for ID8.2 and GD-BSA has binding sites for both ID8.2 and BE2. In preferred embodiments, ID8.2 is used because it binds to soman, sarin, tabun, and VX and BE2 only binds to soman. In some preferred embodiments, both ID8.2 and BE2 are used, increasing the sensitivity of the test. In the presence of the chemical warfare nerve agents, the chemical warfare nerve agents compete for the binding sites of BE2 and ID8.2 antibodies in the blocking antibody zone 906. Since BE2 and ID8.2 bind to the chemical warfare nerve agents, the latex beads pass over the blocking antibody zone 906 and are captured at the test line 905 in the detection zone 908, where they bind to the immobilized Albegone. In a preferred embodiment, the detection zone 908 also includes a control line 904.

If the chemical warfare nerve agent is Soman, Soman binds to both the BE2 and ID8.2 antibodies, and both the blue and red colored latex beads travel to the test line 905, resulting in a red and blue color (making the test line purplish) at the test line 905. If chemical warfare nerve agents other than Soman are present, the chemical warfare nerve agents only bind to the ID8.2 in the antibody zone 906, and the red colored human serum albumin-GD latex beads pass over the blocking antibody zone 906 and get captured at the Albegone test line 905, becoming visible as a red line. In embodiments using fluorescent flash red beads, these beads provide higher sensitivity under fluorescence.

In another general form of this test, the lateral flow immunoassay includes at least a sample application zone 44, a conjugate zone 903 (which may overlap the sample application zone), a hapten zone 906 and a test line 905. In this embodiment, the test line 905 preferably has a binding partner immobilized on it with binding sites to capture the conjugated albumin that comes into the detection zone 908. A human serum albumin or bovine serum albumin with a binding site for the binding partner at the test line is conjugated to a label and preferably included in the conjugate zone 903. In between the sample application zone/conjugate zone 44/903 (preferably close to the sample application zone) and the test line 905 is a hapten zone 906. The hapten zone 906 includes Albegone (or monoclonal or polyclonal antibodies to mammalian albumin). The Albegone binds to mammalian albumin on the conjugated albumin and the adducted target analyte. The target analyte in the sample and the conjugated albumin compete for the binding sites on the Albegone immobilized in the hapten zone 906.

If a sample without the target is added to the assay, Albegone in the hapten zone 906 binds to the conjugated albumin. So, all of the labeled conjugate binds to Albegone in the hapten zone 906. Consequently, no detectable label makes it to the test line 905.

If a sample with the target is added to the assay, the adducted chemical warfare nerve agents compete with the conjugated albumin for the binding sites on the Albegone in the hapten zone 906. So, some of the conjugated albumin is able to travel to the test line 905. The conjugated albumin binds to its binding partner at the test line, creating a detectable label and a positive result at the test line 905.

Unlike other inhibition/competition assays, this assay is a direct correlation of inhibition. Instead of a negative result received in the presence of the target analyte, there is a positive result at the test line 905 in the presence of the target analyte.

In other embodiments, other configurations for the test strip, using some of the elements shown in FIGS. 9-47 (for example the sample compressor and/or the diverting zone) or assay configurations otherwise known in the art, could be used. Albegone (or, in other preferred embodiments, monoclonal or polyclonal antibodies to bovine serum albumin or human serum albumin) is placed at the test line (for example in the assay configurations shown in FIGS. 16, 18, 23A, and 37-47) or, in embodiments where the full sandwich is formed before the sample reaches the test line and tags, such as biotin and avidin, are used (see, for example in the assay configurations shown in FIGS. 19-22, 23B-23D, and 26-36), Albegone (or, in other preferred embodiments, monoclonal or polyclonal antibodies to bovine serum albumin or human serum albumin) acts as the mobile second binding partner for the chemical warfare nerve agent. In embodiments where Albegone is in the hapten zone, Albegone may be placed on the test strip and/or, in configurations with a sample compressor, Albegone may be placed on the sample compressor or on the test strip.

Positive Correlation Point of Care Assay with a Hapten Zone

FIG. 49 shows another embodiment of a direct correlation inhibition assay with a hapten zone. In other embodiments, other configurations for the test strip, as shown in FIGS. 9-47 or otherwise known in the art, could be used with a hapten zone.

In a general form of this test, a lateral flow immunoassay includes a sample analysis device 1000 with at least a sample application zone 44, a conjugate zone 1003 (which may overlap the sample application zone 44), a hapten zone 1006 and a test line 1005. In preferred embodiments, the detection zone 1008 of the device also includes a control line 1004. The test line 1005 preferably has an antigen to capture any conjugated antibody that comes into the test zone 1005. A binding partner to the analyte, for example, an antibody to the analyte, is conjugated to a label and placed in the conjugate zone 1003. When the analyte in the sample sees the conjugated binding partner, the analyte binds to the conjugate binding partner. In between the sample application zone 44/conjugate zone 1003 and the test line 1005 is a hapten zone 1006. The hapten zone 1006 includes the target, or an analog to the target, immobilized in the hapten zone 1006. In some preferred embodiments, rabbit anti-chicken is used at the control line 1004.

If a sample without the target is added to the assay, the conjugate that contains the binding partner for the target does not have anything to bind because there is no target. So, all of the labeled conjugate binds to the target or target analog in the hapten zone 1006. Consequently, no visible label makes it to the test line 1005. In preferred embodiments with a hapten zone 1006, the hapten zone 1006 is not visible to the user because it is fully encompassed by a housing.

If a sample with the target is added to the assay, the conjugate that contains the binding partner for the target binds to the target. Since it is now bound to the target, it can not also bind to the target or target analog in the hapten zone 1006. Consequently, it passes over the hapten zone 1006, travels to the test line 1005, and binds to the immobilized binding partner for the conjugate (which, in preferred embodiments, has been immobilized to the test line using Fclock, see embodiment described further below) on the test line 1005.

Unlike other inhibition/competition assays, this assay is a direct correlation of inhibition. Instead of a negative result received in the presence of the target, there is a positive result at the test line 1005 in the presence of the target analyte.

In one preferred embodiment to test for chemical warfare nerve agents, the lateral flow immunoassay includes at least a sample application zone 44, a conjugate zone 1003 (which may overlap the sample application zone 44), a hapten zone 1006 and a test line 1005. The test line 1005 preferably includes an immobilized rabbit anti-mouse antibody to capture any conjugated mouse antibody that comes into the test zone 1005. A mouse antibody to the chemical warfare agent is conjugated to a label and included in the conjugate zone 1003. When the analyte in the sample sees the conjugated antibody, the analyte binds to the conjugate. The hapten zone 1006 is located in between the sample application zone 44/conjugate zone 1003 and the test line 1005. The hapten zone 1006 includes the chemical warfare nerve agent, or an analog to the chemical warfare nerve agent, immobilized in the hapten zone 1006.

If a sample without chemical warfare nerve agent is added to the assay, the conjugate that contains the binding partner for the chemical warfare nerve agent does not have anything to bind because there is no chemical warfare nerve agent in the sample. So, all of the labeled conjugate will bind to the chemical warfare nerve agent or analog of the chemical warfare agent in the hapten zone 1006. Consequently, no visible label makes it to the test line 1005.

If a sample with a chemical warfare nerve agent is added to the assay, the conjugate that contains the binding partner for the chemical warfare nerve agent binds to the chemical warfare nerve agent. Since it is now bound to the chemical warfare nerve agent, it can not also bind to the chemical warfare nerve agent or analog to the chemical warfare nerve agent immobilized in the hapten zone 1006. Consequently, it passes over the hapten zone 1006, and travels to the test line 1005, where the rabbit anti-mouse antibody (which, in preferred embodiments, has been immobilized to the test line using Fclock, see embodiment described further below) captures the labeled mouse antibody. The Fc portion of the labeled conjugate is captured.

In initial testing, the test was used to detect the presence of blood protein-nerve agent adducts in exposed blood samples. In order to mimic the in vivo exposure as closely as possible, nerve agents stored in organic solvents were spiked in minute quantities into whole blood samples. For performance testing, 40 plasma samples were spiked with Sarin, Soman, Tabun, or VX and 10 normal plasma samples were used as the negative control. The 40 nerve agent-spiked plasma samples included 10 replicates of each agent. At the clinically relevant low-level exposure of 10 ng/ml, the test demonstrated 100% sensitivity for Soman, Tabun, and VX and 80% sensitivity for Sarin. The test demonstrated greater than 97% specificity with 150 blood samples obtained from healthy adults. No cross-reactivity or interference from pesticide precursor compounds was found.

BE2 and ID8.2 monoclonal antibodies were chosen for the assay. The ID8.2 antibody was raised against the methyl phosphoryl portion of Soman. Antibody ID8.2 (ID8) binds specifically to any nerve agent protein adducts in the blood sample. ID8 was developed and raised by the U.S. Army Medical Research Institute of Chemical Defense against the methyl phosphoryl portion of nerve agents.

The ID8.2 antibody is shown in FIG. 5. ID8.2 antibody binds to organophosphate nerve agents, specifically Soman, Sarin, Tabun, and VX. (“Role of immunogen design in induction of Soman-specific monoclonal antibodies”, Johnson J K, Cerasoli D M, Lenz D E. U.S. Army Medical Research Institute of Chemical Defense. Immunology Letters (2005) 96:121-127, herein incorporated by reference). Hybridoma cell lines for these two monoclonal antibodies were further optimized by cloning and recloning. The cloned cell lines were acclimated to grow and produce the highly specific monoclonal antibodies in bioreactors. The cell culture supernatants were purified on a protein G column FIG. 3 diagrams Soman and Soman conjugates that are used as immunogens. The common epitope to all chemical nerve agents is highlighted by the circle. The BE2 and ID8.2 monoclonal antibodies were conjugated to 40 nm colloidal gold nanoparticles and served as the optically detectable red conjugates in the lateral flow point-of-care test. Chicken IgY conjugated colloidal gold served as the control conjugate. Rabbit anti-chicken and rabbit anti-mouse were immobilized at the control and test lines respectively.

The methods and devices described herein use a finger stick blood sample, or plasma sample, and require no prior sample preparation, external blood separation, or extraction steps. In a preferred embodiment, the fingertip is punctured, for example using a lancet. A pipette is held horizontally to collect the blood sample, preferably a 10 μl blood sample. The pipette bulb is pressed to transfer the full amount of the blood sample to the sample application zone of the sample analysis device. The test is then assembled by placing the sample compressor onto the test cassette. The test is then immersed in a buffer vial for approximately 20 seconds to activate lateral flow.

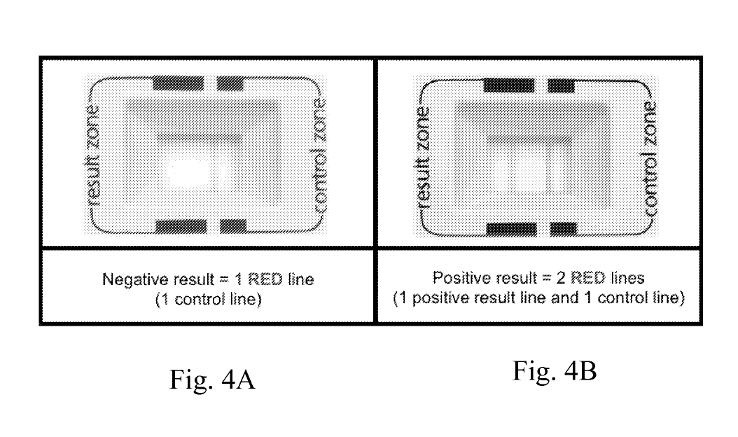

Test results are preferably optically read in 10 minutes. A valid, negative test produces one red line, the control line. A valid, positive test indicating nerve agent poisoning greater than or equal to 10 ng/ml produces a second red test line in addition to the red control line. An example of a negative test result (showing only the single control line) is shown in FIG. 4A. An example of a positive test result, showing the control line plus a positive result at the test line, is shown in FIG. 4B.

### Analytical Specificity

To test analytical specificity, whole blood samples from 150 healthy persons were obtained from a major blood bank. A volume of 20 μl of whole blood was pipetted onto the assay and the test was initiated and interpreted according to the instructions for use.

Testing of whole blood samples from 150 healthy persons demonstrated an analytical specificity of 97.3% (146/150). Four samples were found to induce a false positive result. Retesting of the four presumed false positives resulted in two of the four samples testing as negative, however, this was not considered in the calculation of the specificity.

Analytical Sensitivity

In performance testing, 40 plasma samples were spiked with chemical nerve agents and 10 normal plasma samples were used as the negative control. The 40 nerve agent-spiked plasma samples included 10 replicates of each of the four agents. 20 μl of nerve agent-spiked samples and normal plasma controls were tested.

In performance testing at the clinically relevant low-level exposure of 10 ng/ml, the test demonstrated 100% sensitivity for Soman, Tabun, and VX and 80% sensitivity to Sarin. In Table 1, the accuracy of the optical interpretation of the test at exposure levels of 10 ng/ml, 5 ng/ml and 1 ng/ml are shown. Table 1 shows the analytical sensitivity results (optically read positive results by nerve agent exposure concentration. N=10 per concentration of each agent.) The variation of optically confirmed positivity at nerve agent concentrations below 10 ng/ml are attributed to the difference in binding affinities of ID8.2 in each of the different chemical nerve agents.

| | Concentration of Exposed | |
|---|---|---|
| Nerve Agent | Plasma in ng/ml | Visual Positivity |
| Soman (GD) | 0 | — |
| | 1 | 10% |
| | 5 | 60% |
| | 10 | 100% |
| Sarin (GB) | 0 | — |
| | 1 | 0% |
| | 5 | 80% |
| | 10 | 80% |
| VX | 0 | — |
| | 1 | 50% |
| | 5 | 90% |
| | 10 | 100% |
| Tabun (GA) | 0 | — |
| | 1 | 90% |
| | 5 | 100% |
| | 10 | 100% |

Cross Reactivity with Organophosphate (OP) Pesticides

The assay was tested for cross reactivity with the following pesticides and their metabolites: malathion, ethyl parathion, methyl parathion, organophosphorous pesticide mixture, malathion-o-analog, dimethyl-p-nitrophenylphosphate, and paraoxon. Each pesticide or pesticide metabolite was spiked into normal whole blood at concentrations of 100 ng/ml. Twenty μl of the spiked blood sample was then pipetted directly onto the assay device. The test was initiated, and the results were interpreted at 10 minutes.

No cross reactivity was identified with any of the following OP pesticides: malathion, ethyl parathion, methyl parathion, organophosphorous pesticide mixture, malathion-o-analog, dimethyl-p-nitrophenylphosphate, or paraoxon.

The lack of an accurate diagnosis of low-level chemical nerve agent exposure may result in a delay in, or lack of receipt of, needed treatment. In addition, the use of life-endangering resuscitation drugs or high-risk anesthetics can cause injury or death in those who have been exposed to chemical nerve agents.

Using the devices and methods described herein to test at-risk populations suspected of being exposed to chemical nerve agents may lead to early identification and treatment in the event of low-level or chronic exposure. This is especially true for the testing of the walking well or individuals who are worried about nerve agent exposure to confirm if low level poisoning has occurred. The assay devices in these embodiments provide a low cost, easy to use capability to meet the need to rapidly identify populations that have been exposed, or suspected of exposure, to low levels of Sarin, Soman Tabun, or VX chemical nerve agents.

One preferred kit using the methods and devices described herein includes gloves, an alcohol prep pad, lancet, gauze, one or more pipettes (a sample collector), a bandage, a test cassette, a sample compressor, activating buffer, and instructions for use.

### Use of Fclock to Increase Efficiency

In one preferred embodiment, the competitive inhibition assays described herein use Albegone recombinant protein and/or Fclock protein.

In this embodiment, a competitive inhibition assay uses the recombinant Albegone and Fclock, which have very high binding constants to Albumin and immunoglobulin. Consequently, antibodies can be used in smaller quantities more efficiently (right side up).

Fclock may be used in any of the configurations for the test strip, as shown in FIGS. 9-47 described below or otherwise known in the art to keep the antibody reagents right side up in their locations on the test strip. Similarly, Albegone may be used as a substitute for the first binding partner or the second binding partner in any of the configurations of FIGS. 9-47 described below to detect chemical warfare nerve agents adducted to albumin.

In one preferred embodiment, the strip is a “universal” strip for all inhibition type assays. Only the antibody to be striped on to the Fclock and the latex beads differ from test to test. Since the recombinants are used, there is a greater chance for longer stability and expiration dating. The cost is also reduced because the recombinant can be produced in greater quantities and the critical reagents such as antibodies are efficiently used.

Fclock (disclosed in PCT patent publication WO2011/068538 GENERAL METHOD FOR GENERATING ULTRA-HIGH AFFINITY BINDING PROTEINS, Hammerstedt et al., published Sep. 6, 2011, incorporated herein by reference) is a protein with an inherent capacity to bind the Fc portion of immunoglobulins. FcLock binds immunoglobulins more tightly than protein A and protein G. There is a FcALock, which binds to IgAs and a FcGlock, which binds to IgGs.

As discussed above, Albegone is a recombinant protein that binds to mammalian albumins. Note that Albegone does not bind to egg albumin (for example, Albegone does not bind to chicken IgY, which may be used in the embodiments described herein as a control).

If Fclock is immobilized on a conjugate zone (or any zone with antibody reagents that need to be “right side up”), and then one or more antibodies are striped on top of the Fclock in the same location, the Fc (bottom of the Y shape) portion of the antibody binds to the Fclock and the FAb portion (top of the Y shape) of the antibody is facing up, available for binding to the target or other reagents in the assay. In addition, since Fclock has such tight binding to the antibody, it is not washed off. Using Fclock permits harsher running buffer conditions (e.g. temperature, humidity, etc.) because the binding between the Fclock and the antibody is so stable.

The Fclock may be used in any zone where antibodies are going to be added to the zone.

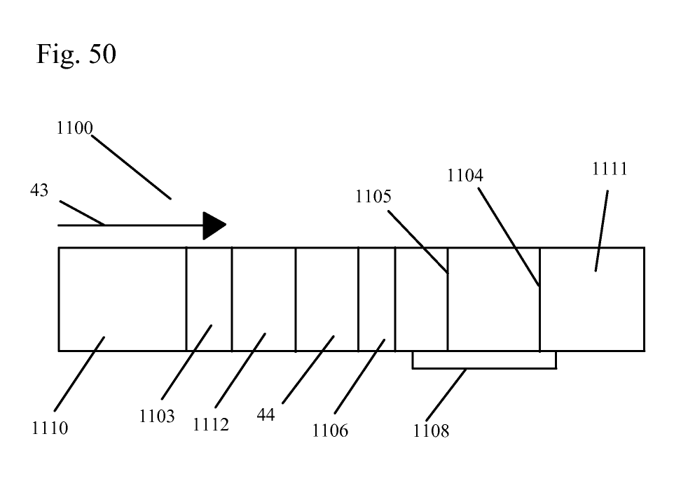

One example of a test strip 1100 using Fclock is shown in FIG. 50. The strip 1100 preferably includes an absorbent/wicking pad 1110, a conjugate pad/zone 1103, a space 1112, a sample application zone 44, a clearance or blocking antibody zone 1106, a test line 1105, a control line 1104 and a waste pad 1111. The test line 1105 and the control line 1104 form the detection zone 1108. While the conjugate zone 1103 is shown upstream of the sample application zone 44 in this embodiment, in other embodiments, the conjugate zone 1103 is downstream of the sample application zone 44. In still other embodiments, the conjugate zone 1103 and the sample application zone 44 overlap.

In one preferred embodiment, the conjugate in the conjugate zone 1103 is preferably MATP-HSA Antigen. This is a soman derivative methyl phosphonic acid, p-aminophenyl 1,2,2,-trimethyl-propyl diester (MATP) bound to human serum albumin. The clearance/antibody zone 1106 includes the BE2 antibody. In a preferred embodiment, the Fclock recombinant protein is placed in the clearance zone 1106. Then, the BE2 antibody is striped in the clearance zone 1106, and attaches to the Fclock recombinant protein. The test line 1105 is preferably Albegone and the control line 1104 is anti-chicken IgY. The Fclock recombinant is used to arrange antibodies in the desired position on the nitrocellulose membrane of the test strip 1100. For example, the Fclock is preferably striped on the conjugate pad/zone 1103 and/or the clearance zone 1106 before the MATP-HSA and BE2 antibodies are added to those respective zones.

In this example, in the absence of soman, the conjugate binds to the BE2 antibody in the clearance/antibody zone 1106. In the presence of soman, the soman competes with the conjugate for the BE2 antibody, so the conjugate travels to the test line 1105, producing a detectable result at the test line 1105.

In the embodiments described above with a blocking antibody zone or a hapten zone, suppose the antibody conjugate is inactive because it is not right side up. The conjugate escapes these zones because the antibodies are not right side up, but then is captured at the test line. This results in false positives. Therefore, Fclock is preferably used in any or all of the conjugate zones, hapten zones, and/or the blocking antibody zones described herein. This increases the efficiency of the assay, since instead of approximately 30-35% of the antibodies being right side up and available for binding, 100% of the antibodies will be available for binding.

In other embodiments, the Fclock is placed on the label, for example colloidal gold. After the Fclock is placed on the label, the antibody is added and the Fclock binds to the Fc portion of the antibody. This ensures that the antibody is “right side up” for use in the assay.

Detection of Hydrolyzed Soman and Sarin with BE2 and ID8 Monoclonal Antibodies

In J K Johnson, D M Cerasoli and D E Lenz, Immunology Letters 96 (2005) 121-127, herein incorporated by reference, the authors stated that the antibodies' inability to bind to hydrolyzed Soman may be accounted for by the fact that hydrolyzed Soman is ionized in water and contains a negative charge.

This implies that the monoclonal antibodies BE2 and ID8.2 are repelled by the negative charge. One preferred embodiment neutralizes the negative charge on hydrolyzed Soman using positively charged reagents. In one preferred embodiment, the positively charged reagent is polylysine. Once neutralized, these metabolites of Soman may bind to antibodies to Soman, for example BE2 and/or ID8.2 antibodies.

Because of the negative charge, Soman and Sarin can bind to reagents such as polylysine and still retain the pinacolyl motif. In one preferred embodiment, polylysine is added to the samples of hydrolyzed Soman and Sarin before testing. For example, polylysine is dried onto the sample application zone 44 where the sample containing hydrolyzed Soman and Sarin is added. This is “in situ” neutralization. In another embodiment, polylysine is added to the running buffer.

In another preferred embodiment, polylysine is conjugated onto colloidal gold or latex beads and the hydrolyzed Soman and Sarin are added to make visual tags that bind to BE2 and ID8.2 immobilized in the nitrocellulose.

In another preferred embodiment, polylysine is immobilized on the nitrocellulose as a test line or as a hapten zone. Hydrolyzed Soman and Sarin is then bound to polylysine and immobilized. Labelled BE2 and/or ID8.2 directly bind to the Soman or Sarin immobilized in the test zone. This serves as a direct sandwich assay for the hydrolyzed Soman and Sarin. This format may also be a part of an inhibition assay to detect metabolites of Soman.

Polylysine may be used in the sample application zone 44 in any of the assay configurations described herein, including FIGS. 9-50. In alternative embodiments, polylysine may be included on any of the test lines, hapten zones, or blocking antibody zones described herein. In preferred embodiments, the polylysine is physically separated from the antibody and sees the hydrolyzed chemical warfare nerve agent (Soman or Sarin) before the antibody sees the chemical warfare nerve agent. In one preferred embodiment, polylysine could be encapsulated, and embedded in a conjugate zone.

### a Direct Sandwich Assay for Urinary Metabolites of CWNAs and Certain Toxic Industrial Chemicals (TICs)

In another preferred embodiment, a direct sandwich assay for urinary metabolites of chemical warfare nerve agents and/or toxic industrial organic chemicals (TICs) occurs inside a microcapillary tube (for example a microcapillary tube from ChromBA Inc., State College, Pa.).

C18 (an octadecyl carbon chain) binds to all organics through hydrophobic interactions. Urinary metabolites of chemical warfare nerve agents are mainly hydrophobic and should bind non-specifically to C18 coated microcapillaries. Suitably labeled specific monoclonal antibodies are then added to interact with the bound metabolites, giving rise to visibly discernable color. These embodiments preferably detect the chemical warfare nerve agents by themselves rather than their adducts to albumin A C18 type matrix effectively captures such small molecular weight organics. This method detects various metabolites of chemical warfare nerve agents as long as specific monoclonal reagents are available. Any organic compound, be it an industrial pollutant or toxic industrial chemical, may be directly detected in this format.

C18 coated microcapillaries are available in various forms from ChromBA (State College, Pa.). One practical form is at the end of a syringe or a pipette tip. Urine containing the chemical warfare nerve agents metabolites is allowed to pass through the microcapillary. All organic compounds adhere or bind strongly to the C18 through hydrophobic bonds. A simple washing step is included to remove all the unbound materials. Specific antibodies, which bind to the metabolites immobilized in the microcapillary tubes bound to C18, conjugated to colloidal gold or dyed latex beads, are added. Another simple washing step removes all the unbound materials, leaving behind a visibly discernable tip in the presence of the chemical warfare nerve agents.

Detecting Chemical Warfare Nerve Agents with C18

C18 (an octadecyl carbon chain) is commercially available in a matrix or gel for HPLC columns Since C18 binds to anything organic, it someone is trying to detect an organic, it can be passed through a C18 column C18 gathers the organic molecules in a sample. In other embodiments, hydrocarbon removal filter materials, which specifically bind to organic molecules, could be substituted for C18. For example, the Emerald 240 Series “Oil Guzzler” Cartridges (Filtration Technology Corporation, Houston, Tex.) could be used.

Raw agents, as well as the metabolites of chemical warfare nerve agents or sulfur mustard are organic in nature. Urine from individuals exposed to chemical warfare nerve agents has these metabolites.

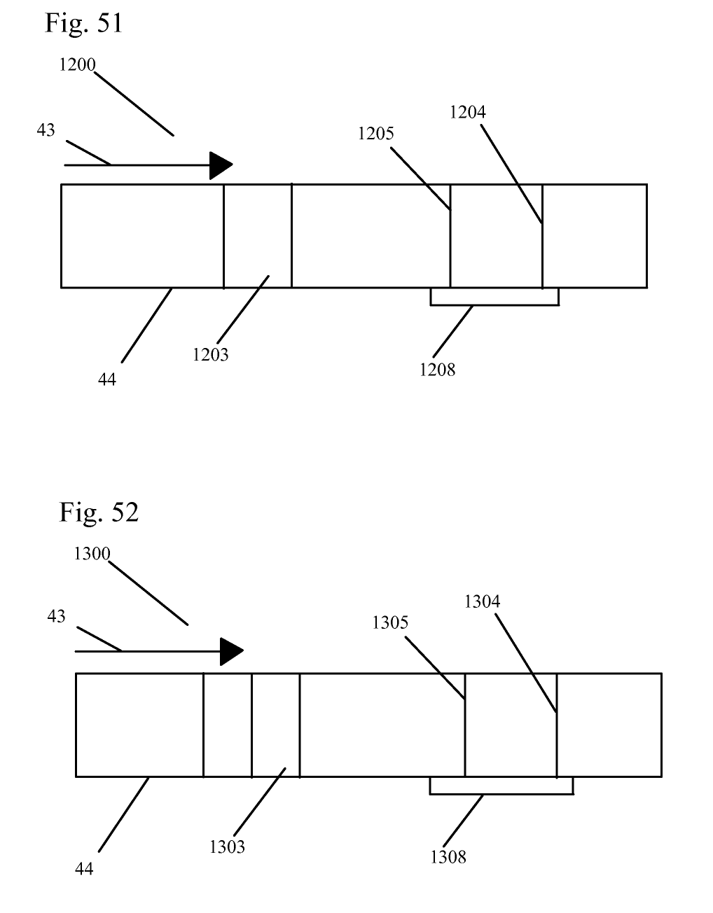

In one preferred embodiment using any of the lateral flow chromatographic test strips, C18 is immobilized as the test line, as shown in FIG. 51. The lateral flow chromatographic test strip 1200 includes at least a sample application zone 44, a conjugate zone 1203 and a test line 1205. While the conjugate zone 1203 is shown downstream of the sample application zone 44 in this figure, alternative configurations, for example the conjugate zone 1203 and sample application zone 44 overlapping or the conjugate zone 1203 being upstream of the sample application zone 44, are also possible.

The test line 1205 preferably has C18 or a similar material immobilized on it to capture any organics that travel to the detection zone 1208. If a sample without the target organic analyte is added to the assay, the conjugate in the conjugate zone 1203 does not bind to the organic analyte, and no labeled conjugate binds to C18 in the test zone 1205. If a sample with the target organic analyte is added to the assay, the labeled conjugate binds to the organic analyte, and the organic analyte binds to C18 in the test zone 1205, resulting in a visible line (a positive result) in the test zone 1205. In a preferred embodiment, the detection zone 1208 also includes a control line 1204.

In one preferred embodiment, an aqueous sample, such as urine, is added to the test strip 1200. The organic analytes that are to be tested for are chemical warfare nerve agents. Visual and/or fluorescent labels conjugated to chemical warfare nerve agent antibodies (for example BE2 and/or ID8.2) act as the conjugate. The binding of the antibody conjugate to the chemical warfare nerve agent metabolites bound to C18 on the test line 1205 indicates the presence of the metabolites (or raw agents) directly. The sensitivity of the assay may be adjusted either by fluorescence or any other amplification methods.

The visual test can be made more sensitive by incorporating fluoresceinated beads. Any of the assays and methods of US Patent Publication No. 2009/0289201, published Nov. 26, 2009, entitled “COMBINED VISUAL/FLUORESCENCE ANALYTE DETECTION TEST”, incorporated herein by reference, may be used to enhance the sensitivity of the test.

In alternative embodiments, C18 may be used at the test line in inhibition assays.

While FIG. 51 shows a particular configuration for an assay using C18, C18 may be used as the test line in any of the assay configurations shown in FIGS. 16, 18, 23A, and 37-47 described below. In other preferred embodiments where both the first and the second binding partners are upstream of the test zone, and the methods and devices include a tag and an immobilized tag (for example in the assay configurations shown in FIGS. 19-22, 23B-23D, and 26-36 described below), C18 replaces the first or second binding partner in the assay.

As another example, benzene is the organic analyte of interest and the assay is a competitive inhibition assay. In this embodiment, shown in FIG. 52, the lateral flow chromatographic test strip 1300 includes at least a sample application zone 44, a competition zone 1303 and a test line 1305. While the competition zone 1303 is shown downstream of the sample application zone 44 in this figure, alternative configurations, for example the competition zone 1303 and sample application zone 44 overlapping or the competition zone 1303 being upstream of the sample application zone 44, are also possible.

The test line 1305 preferably has C18 or a similar material immobilized on it to capture any organics that travel to the detection zone 1308. The competition zone 1303 includes benzene tagged with a label. If a sample without benzene is added to the assay, the labeled benzene in the competition zone 1303 binds to C18 at the test line 1305. If the sample contains benzene, the benzene in the sample competes with the labeled benzene from the competition zone 1303, so there is less of the label at the C18 test line 1305. In a preferred embodiment, the detection zone 1308 also includes a control line 1304.

These embodiments could be used with any type of sample, for example fluid samples from biologics, soil or water.

This method and device is preferably used to detect urinary metabolites of chemical warfare nerve agents from humans or animals. In other embodiments, this method and device are used to detect organic Toxic Industrial Chemicals (TICs).

Any lateral flow assay format known in the art may be used to detect the chemical warfare nerve agents or other organic analytes in the different assay embodiments described herein. The assay formats described below are particularly useful for sandwich assays, but components of many of them could alternatively be used in the direct correlation inhibition assays described herein, or inhibition assays using C18 in the assays.

U.S. Published Patent Application No. 2005/0175992 discloses a method for detecting targets, such as pathogens and/or allergy-associated components, in a human body fluid where the body fluid sample is collected by a collection device, such as a swab member. The samples are transferred from the swab member to a sample analysis device, on which an analysis of the targets can occur by immunochemical or enzymatic means. The test result is capable of being displayed within a very short period of time and can be directly read out by the user.

The chromatographic test strip shown in FIGS. 9 through 12 includes a plurality of different strip materials. The device preferably includes an absorbent pad 1, an application zone 2, a detection zone 3, and a waste zone 4. The strip materials are arranged on an adhesive plastic backing 5. The absorbent pad 1 is provided in this example for adding an elution medium in order to facilitate the transfer of the sample to the detection zone 3. US Patent Publication No. 2007/0059682, describes methods to increase specificity of lateral flow immunoassays. These methods could also be used in combination with the embodiments described herein.

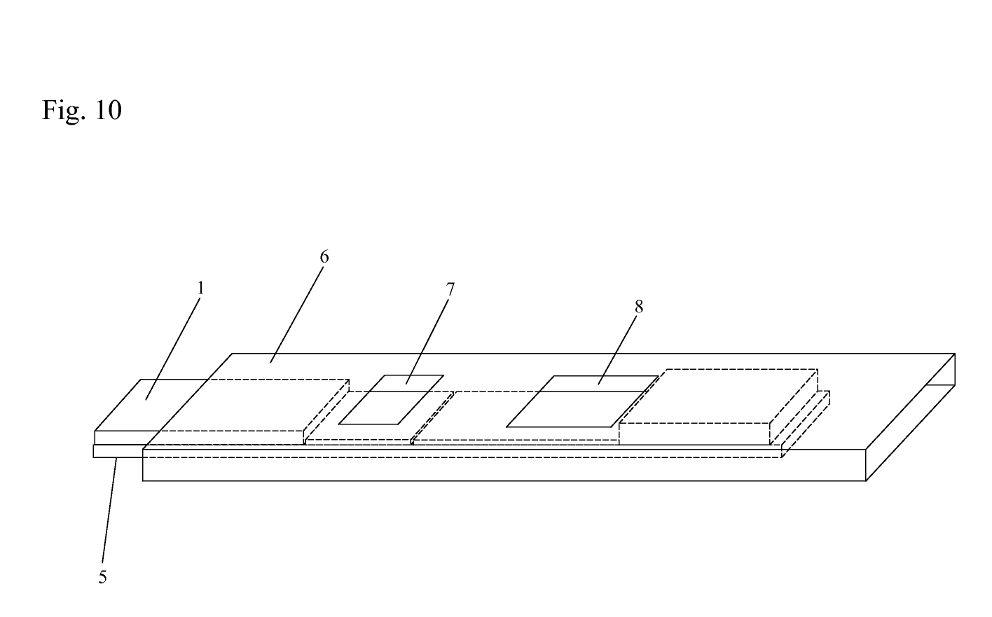

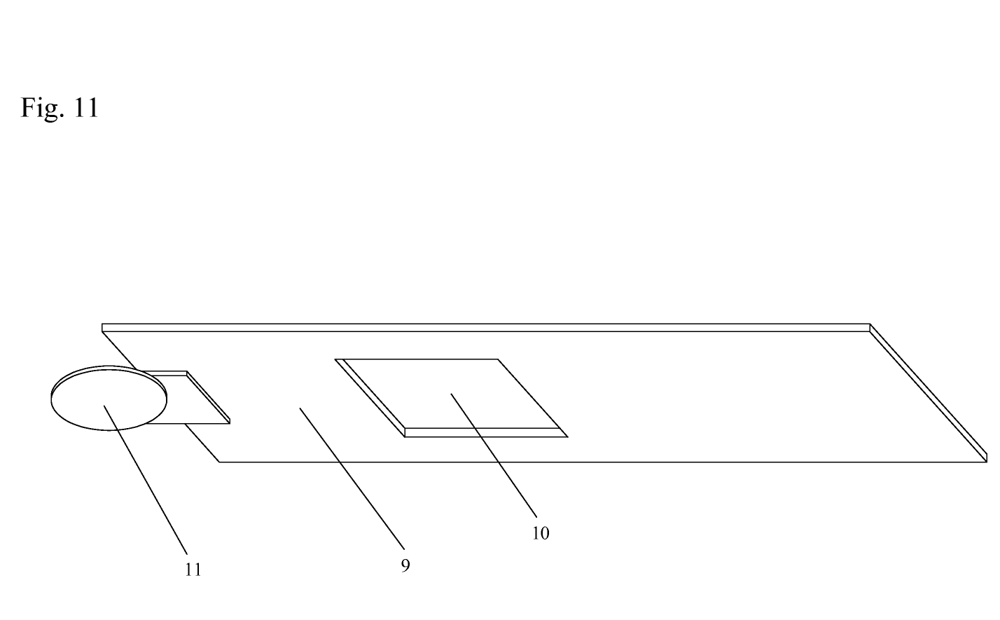

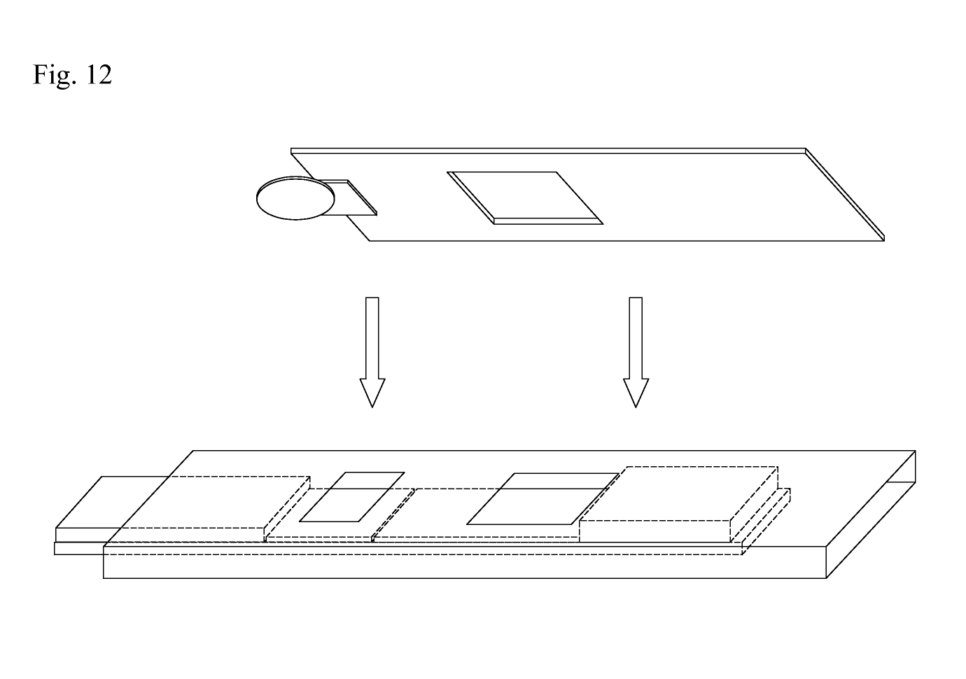

FIG. 10 shows a housing 6, which is preferably plastic, containing the strip as shown in FIG. 9. A sample application window 7 brings a collection device into contact with the strip. The test result is displayed in the read out window 8. FIG. 11 shows the collection device for collecting a sample. In one example, the collection device is a swab member. The collection device includes a body 9, which is preferably plastic, with a sample collection material 11 fixed on it and an opening 10 corresponding to a read out window when the collection device is operatively in contact with a test strip. FIG. 12 shows a test kit, which includes the sample analysis device of FIGS. 9 and 10 and the collection device of FIG. 11.

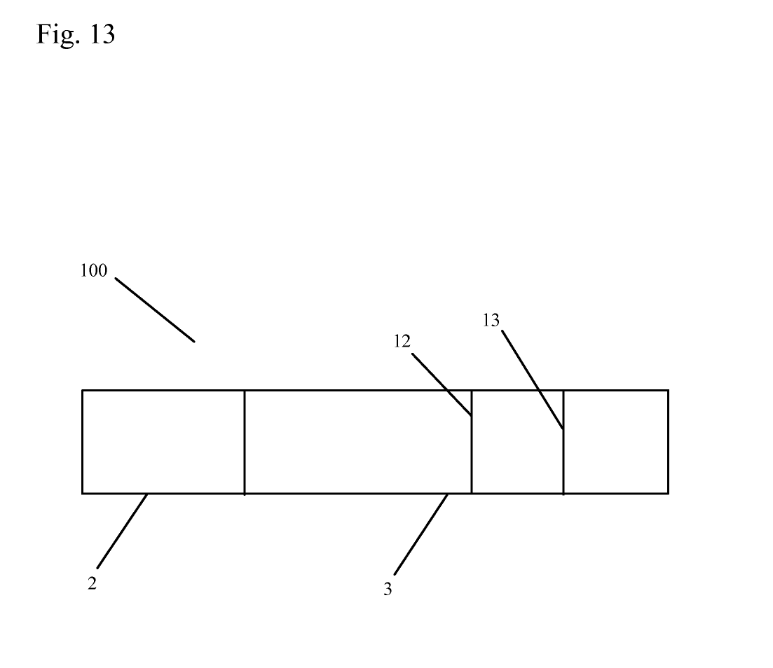

In a method of the invention, it is possible to make use of different biochemical testing procedures to detect constituents on one or several biochemical binding reactions. In a preferred embodiment, as shown in FIG. 13, the chromatographic test strip 100 includes an application zone (or sample zone) 2. The sample is applied to the application zone 2.

The test strip also includes a detection zone 3. The detection zone 3 includes at least one test zone 12, which is preferably a test line. Although only one test line is shown in the figure, multiple test lines are within the spirit of the invention. In some embodiments where there are multiple targets, the presence of each target preferably corresponds to a separate test line. In other embodiments where there are multiple targets, the presence of multiple targets may be indicated on the same test line such that the presence of more than one target has different characteristics than the presence of a single target. For example, the presence of multiple targets on the same test line may be visually indicated by a different color than the presence of each of the targets alone. The test strip 100 also preferably includes at least one control zone 13, which is preferably a control line. As shown in FIG. 13, the control zone 13 is preferably downstream of the test zone 12. However, in other embodiments, the control zone 13 may be located upstream of the test zone 12.

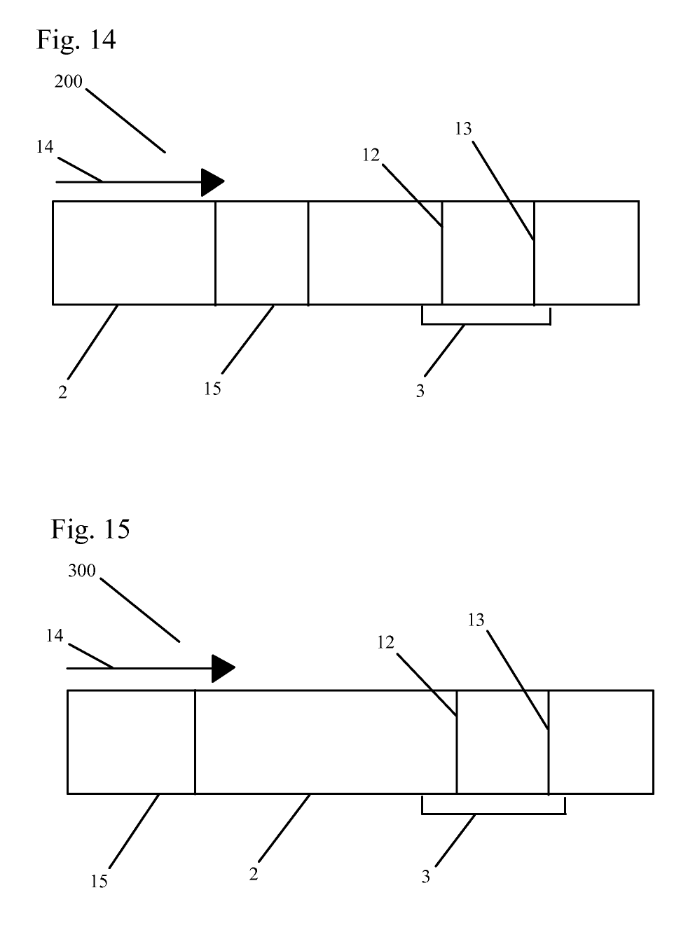

FIGS. 14 and 15 show chromatographic test strips 200, 300 with a test line 12 that detects the presence of a target. The sample is applied to the application zone 2 of the chromatographic test strip 200, 300. The sample is eluted in the direction of the arrow 14, preferably with the aid of an elution medium. As shown in FIG. 14, the sample then passes a conjugate zone 15 containing at least one labeled binding partner that is eluted by and then able to migrate with a sample transport liquid (e.g. a buffer solution). Alternatively, as shown in FIG. 15, the conjugate zone 15 is located upstream of the sample application zone 2 such that the labeled binding partners in the conjugate zone 15 are eluted by the sample transport liquid and travel to the sample. The labeled binding partner is capable of specifically binding to a target/analyte of interest, to form a complex which in turn is capable of specifically binding to another specific reagent or binding partner in the detection zone. Although not shown in these Figures, an absorbent pad, as well as other known lateral flow immunoassay components including, but not limited to, a waste zone, a carrier backing, a housing, and an opening in the housing for result read out, may optionally also be a component of the test strip 200, 300 in these embodiments.

The test strip 200, 300 also includes a detection zone 3 containing a section for detection of the target, e.g. a test line 12, including an immobilized specific binding partner, complementary to the reagent complex formed by the target and its labeled binding partner. Thus, at the test line 12, detection zone binding partners trap the labeled binding partners from the conjugate zone 15 along with their bound targets. This localization of the target with its labeled binding partners gives rise to an indication at the test line 12. At the test line 12, the presence of the target is determined by qualitative and/or quantitative readout of the test line 12 indication resulting from the accumulation of labeled binding partners.

Optionally, the detection zone 3 may contain further test lines to detect other targets, as well as a control line 13. The control line 13 indicates that the labeled specific binding partner traveled through the length of the assay, even though it may not have bound any targets, thus confirming proper operation of the assay. As shown in FIGS. 14 and 15, the control zone 13 is preferably downstream of the test line 12. However, in other embodiments, the control zone 13 may be located upstream of the test line 12.

In a preferred embodiment, the control line 13 includes an antibody or other recombinant protein which binds to a component of the elution medium or other composition being used in the test.

While assay configurations with sample collectors, such as swab members, are described herein, the embodiments described herein preferably directly obtain a blood or plasma sample (for example by a finger stick) and do not require a swab member or any pretreatment before being transferred to the lateral flow device.

FIG. 16 shows a sample analysis device (test strip) 20 and a sample collector 21. The sample collector 21 may be any type of sample collector 21 known in the art, for example the sample collector 21 could be a swab member. The sample 22 may include the analyte 23, as well as interfering particles 25 (which may include interfering proteins or interfering genes) and other interfering particles or cell debris 24. The sample analysis device 20 includes a conjugate zone 15 upstream of the sample application zone 2 in this figure. Although the conjugate zone 15 is shown upstream of the sample application zone 2 in this figure, the conjugate zone 15 may alternatively overlap the sample application zone 2 or be downstream of the sample application zone 2 within the spirit of the present invention. The sample application zone 2 is also a microfiltration zone, which preferably filters out cell debris and interfering particles 24 that are in the sample 22.

The conjugate zone 15 preferably includes both a mobile conjugate 26, which includes a portion that binds to the analyte 23 and a detectable label, and a control zone binding partner 27 with a detectable label, which may be, for example, a control zone antibody with a visual label. In some embodiments, the mobile conjugate is a test antibody conjugate with a visual label. The control zone binding partner 27 binds with an immobilized binding partner for it in the control zone 13 and indicates whether the test has run correctly. If the analyte 23 is present in the sample 22, the analyte 23 binds to the conjugate 26, and the conjugate 26—analyte 23 complex travel to the test zone 12 in the detection zone 3. The analyte 23 then binds to an immobilized binding partner 28 for the analyte 23, to form the full “sandwich” in a sandwich-type assay.

The transfer of the sample from the sample collector 21 to the sample application zone 2 on the sample analysis device is preferably a direct transfer, i.e. the transfer takes place without pretreatment of the sample on the sample collector 21. In embodiments without pretreatment of the sample or the sample collector 21, pressure 17 is applied and microfiltration occurs in the region where the sample collector fleece directly contacts the fleece on the sample analysis device 20. The fibers of the fleece interlock to form a grating or physical interference. Thus, larger elements contained in the sample, for example cell debris and interfering particles 24 are held back and not eluted.

The sample application device 20 preferably also includes a blocking zone 18 that includes one or more capturing reagents. This blocking zone 18 captures interfering proteins and/or genes 25 that may be in the sample 22. Capture of an interfering substance 24, 25 by one or more capturing reagents occurs when the capturing reagent interacts in some manner with the interfering substance to keep the interfering substance from interfering with the detection of the analyte. While a blocking zone 18 is shown in FIG. 16, the capturing reagents may be located in a capturing zone 18 made of materials that allow the capturing reagents to be mobile, in the elution medium, mixed and dried with the reagents, incorporated into the sample application zone, incorporated into the sample collector fleece material, and/or immobilized on an immobilizing material (for example, nitrocellulose) either as a line or a zone. Any of these or any combination of these may be used in the embodiments of the present invention, depending on the test and sample matrix.

The sample analysis device 20 also optionally includes an absorbent pad 1 upstream of the conjugate zone 15 and the sample application zone 2. Buffer is added and travels in the direction of the arrow 14 to elute the test components, including the sample 22, the conjugate 26, and the control zone binding partner 27, to the detection zone 3. The sample analysis device 20 also preferably includes a waste pad 4 at the downstream end of the device 20. The sample analysis device 20 may also optionally include a backing 5.

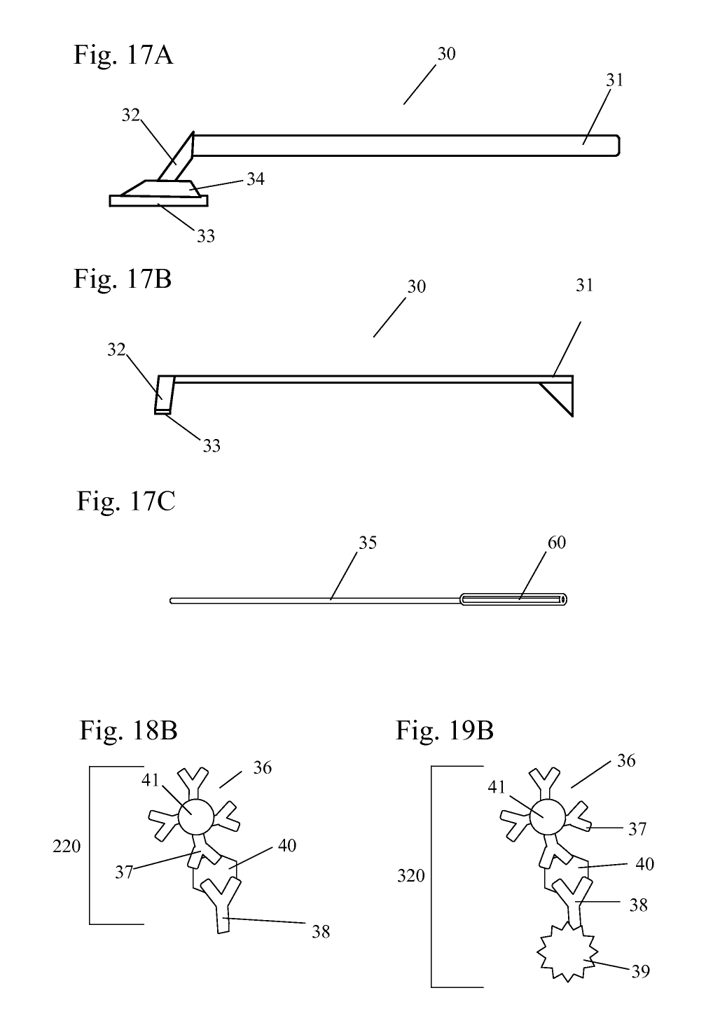

The devices and methods of the present invention preferably include a sample compressor 30. Some schematic examples of sample compressors 30 that could be used are shown in FIGS. 17A and 17B. The sample compressors 30 preferably include a handle 31, an extended portion 32, and a pad portion 33. In some designs, the sample compressor includes additional sections, such as a ledge portion 34 that the pad portion 33 is placed upon. While specific examples are shown in FIGS. 17A and 17B, any sample compressor 30 that is able to exert pressure to transfer one or more components of the assay and the sample to the sample analysis device could be used in the embodiments of the present invention. In preferred embodiments, the conjugate 36 is pre-loaded and dried onto a pad 33 that forms the conjugate zone. In some preferred embodiments, a labeled control 61 that is able to complex with a binding partner at the control zone is also pre-loaded and dried onto the pad 33 of the sample compressor 30. In other preferred embodiments, the second binding partner 38 for the analyte is located on the pad 33. Any combination of the conjugate 36, the second binding partner 38, or the control zone binding partner 61 may be on the pad portion 33 of the sample compressor 30.

FIG. 17C shows an example of a sample collector 35. In this example, the sample collector 35 is a swab member. The sample collector 35 preferably includes a sample collection portion 60, which is preferably made of fleece-type materials. In some embodiments, the sample collector 35 is sterile.

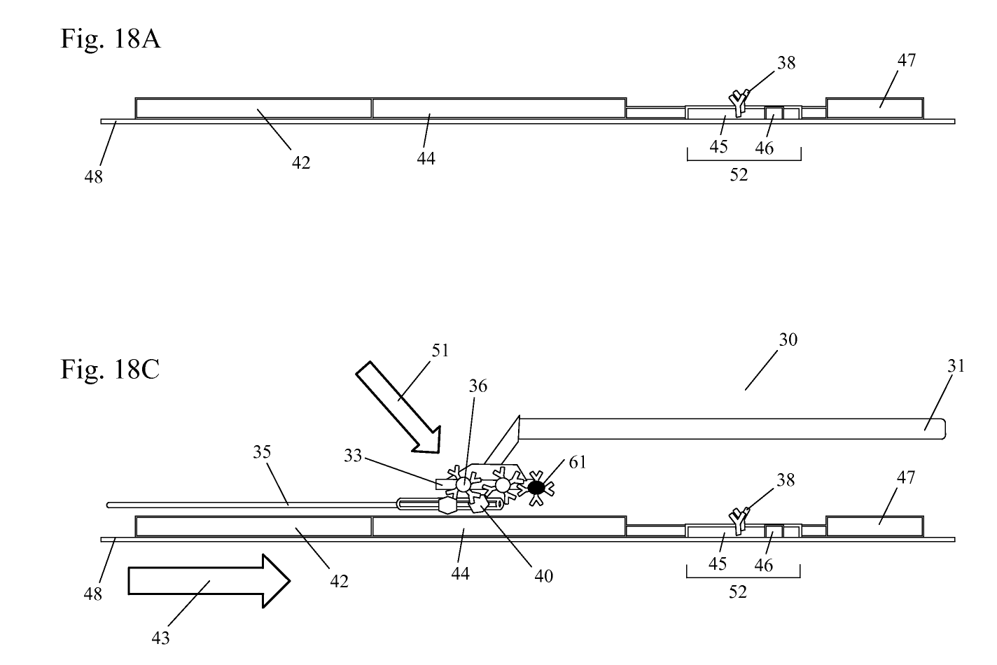

FIGS. 18A through 18C show one embodiment of a system with a sample compressor 30, a sample collector 35, and a sample analysis device (a test strip in the figure). The test strip preferably includes an absorbent pad 42, a sample application zone 44, a detection zone 52, and an optional waste pad 47. The test strip also preferably includes a carrier backing 48. The detection zone 52 preferably includes a test zone 45, which includes an immobilized binding partner 38 for the analyte 40, as well as a control zone 46. In this embodiment, the conjugate 36 is on the sample compressor 30. The first binding partner 37, which is part of the conjugate 36, from the sample compressor 30 binds the analyte 40 in the test sample to form a half sandwich, which is then transported to the second binding partner 38 which is immobilized in a test zone 45. The full sandwich 220 that forms between the portion 37 of the conjugate 36 that binds to the analyte 40, the analyte 40, and the second binding partner 38 is shown in FIG. 18B. In preferred embodiments, the pad 33 on the sample compressor 30 also includes a control zone binding partner 61 with a detectable label. The control zone binding partner 61 complexes with its binding partner in the control zone 46. Including the control zone binding partner 61 on the sample compressor 30, instead of on the test strip or in the buffer as known in the prior art, permits the user to be sure that the components on the sample compressor 30, which, in this embodiment include both the conjugate 36 and the control zone binding partner 61, have effectively transferred to the sample analysis device and thus ensures proper operation of the system.

In one example, both the first binding partner 37 and the second binding partner 38 are different antibodies to the analyte. The control zone binding partner 61 is also preferably an antibody, and its binding partner at the control zone is an antigen (or vice versa). In other embodiments, specific binding partners may also be antigens capable of binding to antibodies against the analyte. Other types of binding partners are bioorganic macromolecules like aptamers or receptors, or nanoparticles. The device shown in FIGS. 18A and 18C of the present invention can be used for any binding assays, and can avoid the use of antibody/antigens, for example, in ligand-receptor binding assays and enzyme-substrate binding assays.

In operation, the sample collector 35 is placed such that the sample is directly above the sample application zone 44. In some embodiments, placement of the sample collector 35 above the sample application zone 44 is not simultaneous with placement of the sample compressor 30. In other words, in these embodiments, some of the sample is transferred to the sample application zone 44 before the sample compressor 30 is added to the vertical stack.

The sample compressor 30 exerts pressure 51 on the sample collector 35, using pressure to transfer the sample, including the analyte 40 (if present), and the conjugate 36 onto the sample application zone 44. If there is also a control zone binding partner 61 on the sample compressor 30, the control zone binding partner 61 is also transferred. Note that the transfer is due to pressure, not due to flow or capillary action. Then, buffer 43 is added to permit flow of the conjugate 36—analyte 40 complex (if present) to the detection zone 52. An immobilized binding partner 38 in the test zone 45 then binds the analyte, forming the complete sandwich. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

In the embodiment of FIGS. 18A-18C, as well as the embodiments of FIGS. 19A through 52, there may also optionally be A blocking zone that includes capturing reagents, similar to the zone discussed with respect to FIG. 16.

In some preferred embodiments using tags, the detection zone includes an antibody against the tag. The antibody may be a monoclonal, polyclonal or single domain antibody. For example, when the tag is biotin, an anti-biotin antibody is immobilized in the test zone instead of avidin, neutravidin, or streptavidin.

FIGS. 19A through 19C show an example of an embodiment of the system with a sample compressor 30, a sample collector 35, and a sample analysis device (a test strip in the figure). Similar to FIG. 18A-18C, the test strip preferably includes an absorbent pad 42, a sample application zone 44, a detection zone 52, and an optional waste pad 47. The test strip also preferably includes a carrier backing 48. In this embodiment, the entire sandwich (first binding partner 37—analyte—40—second binding partner—38) forms in the sample application zone 44 (preferably before the addition of buffer). In some embodiments, placement of the sample collector 35 above the sample application zone 44 is not simultaneous with placement of the sample compressor 30. In other words, in these embodiments, some of the sample is transferred to the sample application zone 44 before the sample compressor 30 is added to the vertical stack.

The test zone 45 in this embodiment includes an immobilized tag 50 that binds to the tag 39 of the second binding partner 38. In this embodiment, a first binding partner 37, which is part of the conjugate 36 and is preferably pre-loaded and dried on the pad 33 of the sample compressor 30, binds the analyte 40 in the test sample to form a half sandwich. The second binding partner 38 in this embodiment is also preferably pre-loaded and dried on the pad 33 of the sample compressor. The second binding partner 38 also includes a tag 39.

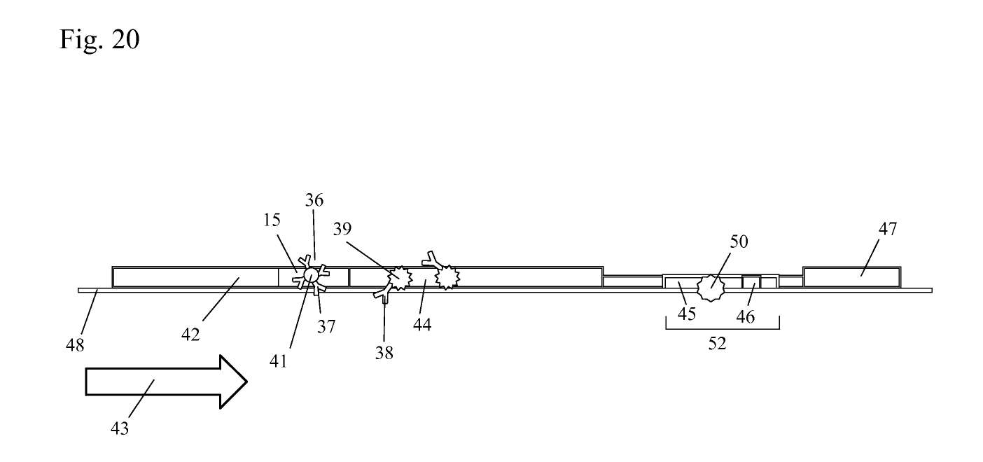

The full sandwich 320 that forms between the binding partner 37 of the conjugate 36, the analyte 40, and the second binding partner 38 in this embodiment (as well as the embodiments in FIGS. 20, 21A-21B, 22A-22B, 23B, 23C, and 23D) is shown in FIG. 19B. In preferred embodiments, the pad 33 on the sample compressor 30 also includes a control zone binding partner 61 (shown in FIG. 18C) with a detectable label. The control zone binding partner 61 complexes with its binding partner in the control zone 46. Including the control zone binding partner 61 on the sample compressor 30, instead of on the test strip or in the buffer as known in the prior art, permits the user to be sure that the components on the sample compressor 30, which include both the conjugate 61 and the control zone binding partner 61, have effectively transferred to the sample analysis device and thus ensures proper operation of the system.

In one example, both the first binding partner 37 and the second binding partner 38 are different antibodies to the analyte. The control zone binding partner 61 is also preferably an antibody, and its binding partner at the control zone is an antigen (or vice versa). In other embodiments, specific binding partners may also be antigens capable of binding to antibodies against the analyte. Other types of binding partners are bioorganic macromolecules like aptamers or receptors, or nanoparticles. The device shown in FIGS. 19A-19C of the present invention can be used for any binding assays, and can avoid the use of antibody/antigens, for example, in ligand-receptor binding assays and enzyme-substrate binding assays.

In one preferred embodiment, the second binding partner 38 is tagged with biotin 39. In embodiments where the tag 39 on the second binding partner 38 is biotin, the immobilized tag 50 in the detection zone is preferably avidin, neutravidin, or streptavidin. In other embodiments, the second binding partner 38 is tagged 39 with avidin, neutravidin, or streptavidin. In these embodiments, the immobilized tag 50 in the detection zone 52 is preferably biotin. Alternatively, the tag 39 on the second binding partner 38 may be a lectin and the immobilized tag 50 may be a glycosyl moiety. For example, in some embodiments, the lectin is the Garden pea Lectin and the glycosyl moiety is an erythrocyte glycosyl unit. The tag on the second binding partner and the immobilized tag may be reversed within the spirit of the present invention. For example, the glycosyl moiety may be the tag on the second binding partner, with an immobilized lectin tag in the detection zone. In other embodiments, other receptors and ligands may be used for the tags.

In operation, the sample collector 35 is placed such that the sample is directly above the sample application zone 44. The sample compressor 30 exerts pressure 51 on the sample collector 35. The pressure transfers the sample (including the analyte 40, if present), the conjugate 36, and the tagged second binding partner 38 onto the sample application zone 44. If there is also a control zone binding partner 61 on the sample compressor 30, the control zone binding partner 61 is also transferred. Note that the transfer is due to pressure, not due to flow or capillary action. Then, buffer 43 is added to permit flow of the conjugate 36—analyte 40 (if present)—second binding partner 38 complex (a complete sandwich) to the detection zone 52. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

In other embodiments, the conjugate zone can contain both the binding partners for the analyte in the sample to form a “full sandwich”. One of the binding partners preferably has a suitable marker such as biotin, avidin, lectin, a glycosyl moiety, a specific ligand, or a specific receptor. The other can be conjugated to the appropriate nanoparticles as mentioned below. The full sandwich is then captured at the test zone where the binding partner of the suitable marker, including, but not limited to, avidin for biotin, biotin for avidin, glycosyl moiety for lectin, lectin for the glycosyl moiety, a receptor for the ligand, or a ligand for the receptor, is immobilized.

FIG. 20 shows an example of a test strip in an embodiment of the present invention. The test strip of FIG. 20 is similar to the test strip of FIG. 19A in that the test zone 45 in this embodiment includes an immobilized tag 50 that binds to the tag 39 of the second binding partner 38. The test strip differs from the test strip of FIG. 19A in that the second binding partner 38 is pre-loaded and dried on the sample application zone 44 of the test strip, while the labeled conjugate 36 is pre-loaded and dried onto a labeled conjugate zone 15 upstream of the sample application zone 44.

Alternatively, the second binding partner 38 and/or the labeled conjugate zone 15 may be located anywhere on the test strip upstream of the detection zone 52 including, but not limited to, overlapping the sample application zone 44, upstream of the sample application zone 44, or between the sample application zone 44 and the detection zone 52. In one preferred embodiment, approximately 75-80% of the labeled 41 conjugate 36 is upstream of the sample application zone (with approximately 20-25% of the labeled conjugate 36 overlapping the sample application zone 44) and approximately 75-80% of the second binding partner 38 is located downstream of the sample application zone 44 (with approximately 20-25% of the second binding partner overlapping the sample application zone 44). Although not preferred, in other embodiments, either the labeled conjugate 36, the second binding partner 38, or both may be located in the buffer or pre-mixed with the sample before the sample is added to the test strip. In still other embodiments, any or all of the components could overlap the detection zone 52.

In some embodiments, both the first binding partner 37 and the second binding partner 38 are different antibodies to the analyte 40. In other embodiments, specific binding partners may also be antigens capable of binding to antibodies against the analyte. Other types of binding partners are bioorganic macromolecules like aptamers or receptors, or nanoparticles. The device shown in FIG. 20 can be used for any binding assays, and can avoid the use of antibody/antigens, for example, in ligand-receptor binding assays and enzyme substrate binding assays.

In operation, a sample collector containing the sample is placed such that the sample is directly above the sample application zone 44. In preferred embodiments, the sample has not been subject to pretreatment prior to application to the test strip. Instead, the sample is still in its native form.

The sample is transferred to the sample application zone 44 of the test strip. A sandwich forms with the labeled conjugate 36 as one piece of bread and the second binding partner 38 as a second piece of bread, with the analyte 40 in between them, when the three components come into contact with each other during flow 43. The labeled conjugate 36—analyte 40 (if present)—second binding partner 38 complex (a complete sandwich) flow to the detection zone 52. An immobilized tag 50 in the test zone 45 then binds the tag 39. Since the labeled conjugate 36 includes a label 41, the complex that forms is detectable and indicates a positive result. Proper operation of the test also results in a detectable positive result in the control zone 46, preferably due to the interaction between a control line binding partner and its immobilized partner in the control zone 46.

In another embodiment, the two binding partners for the analyte are located in such a way to achieve a “vertical sandwich” where the sample binds with the conjugate being compressed from the second plane and can bind simultaneously or concurrently with the other binding partner located on the strip in the plane of the strip. Thus a sandwiching of the analyte in the sample is achieved by binding to the partner from the conjugate delivered from above the plane of the strip and binding to the second binding partner located on the plane of the strip below the sample delivering material.

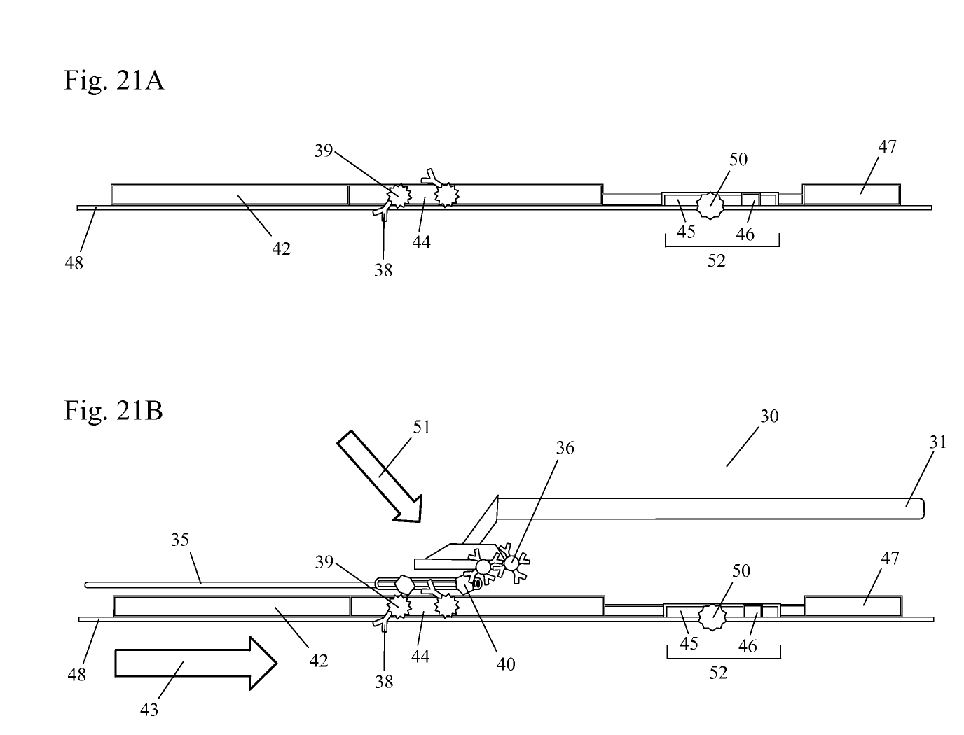

FIGS. 21A and 21B show another example of an embodiment of the system with a sample compressor 30, a sample collector 35, and a sample analysis device (a test strip in the figure). FIGS. 21A and 21B differ from FIGS. 19A and 19C in that the second binding partner 38 in this embodiment is preferably pre-loaded and dried on the sample application zone 44 of the test strip. The second binding partner 38 also includes a tag 39. Alternatively, the second binding partner 38 in this embodiment may be located anywhere on the test strip upstream of the detection zone including, but not limited to, overlapping the sample application zone, upstream of the sample application zone, and between the sample application zone and the detection zone. Similar to the embodiment shown in FIGS. 19A and 19C, in this embodiment, the entire sandwich (first binding partner 37—analyte 40—second binding partner 38) forms in the sample application zone 44.

In preferred embodiments, the pad 33 on the sample compressor 30 also includes a control zone binding partner 61 (shown in FIG. 18C) with a detectable label. The control zone binding partner 61 complexes with its binding partner in the control zone 46. Including the control zone binding partner 61 on the sample compressor 30, instead of on the test strip or in the buffer as known in the prior art, permits the user to be sure that the components on the sample compressor 30, which include both the conjugate 61 and the control zone binding partner 61, have effectively transferred to the sample analysis device and thus ensures proper operation of the system.

In one example, both the first binding partner 37 and the second binding partner 38 are different antibodies to the analyte. The control zone binding partner 61 is also preferably an antibody, and its binding partner at the control zone is an antigen (or vice versa). In other embodiments, specific binding partners may also be antigens capable of binding to antibodies against the analyte. Other types of binding partners are bioorganic macromolecules like aptamers or receptors, or nanoparticles. The device shown in FIGS. 21A-21B of the present invention can be used for any binding assays, and can avoid the use of antibody/antigens, for example, in ligand-receptor binding assays and enzyme-substrate binding assays.

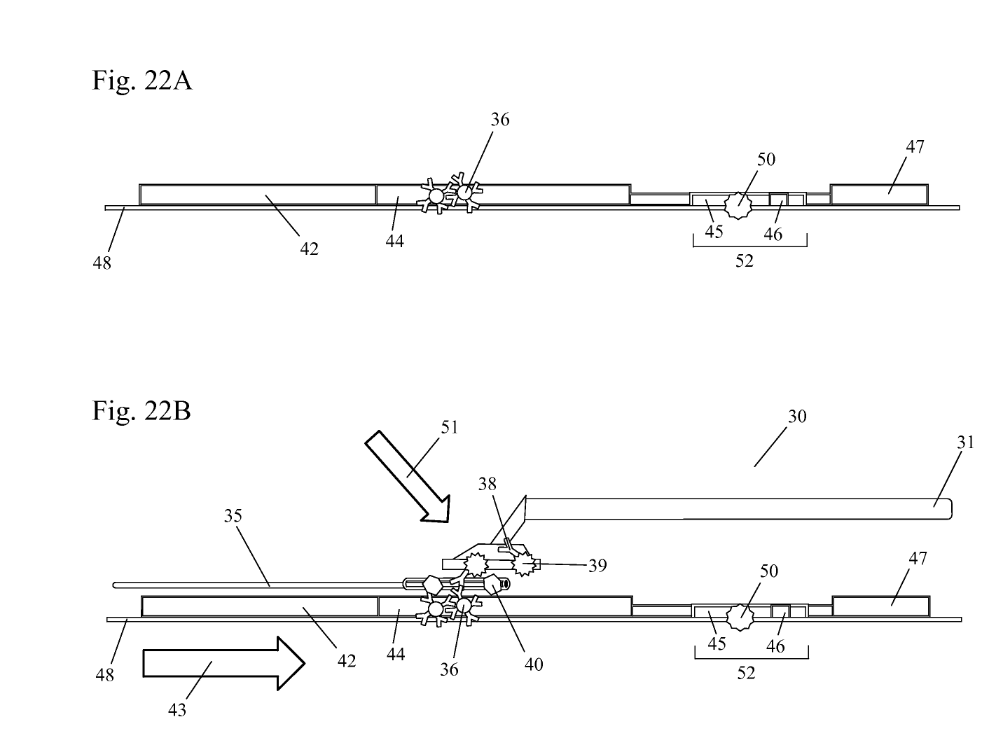

FIGS. 22A and 22B show another embodiment of the present invention, where the sample compressor 30 includes the second binding partner 38 for the analyte 40, coupled with a tag 39, and the test strip includes the conjugate 36, which includes both a first binding partner 37 for the analyte 40 and a detectable label 41, and the immobilized tag 50 that binds to the tag on the second binding partner in the test zone 45. This embodiment operates similarly to the embodiment described with respect to FIGS. 21A and 21B, except that the “vertical” sandwich forms with the second binding partner 38 as the top piece and the conjugate 36 as the bottom piece, with the analyte 40 in between them. Alternatively, the conjugate 36 in this embodiment may be located anywhere on the test strip upstream of the detection zone including, but not limited to, overlapping the sample application zone, upstream of the sample application zone, or between the sample application zone and the detection zone.

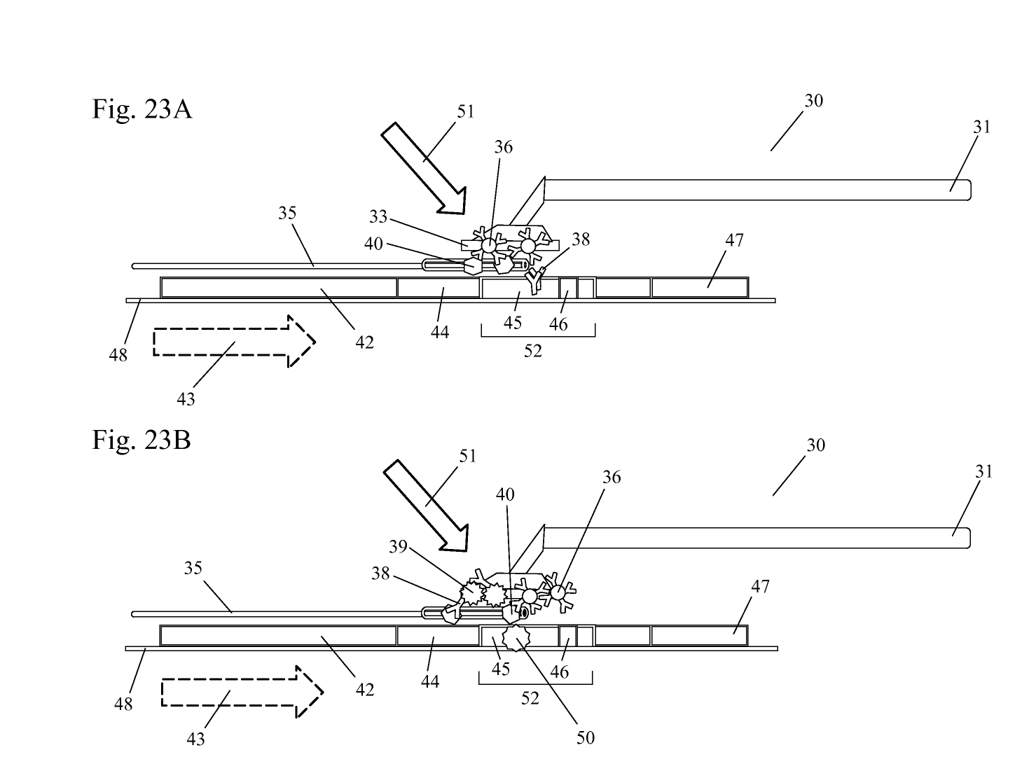

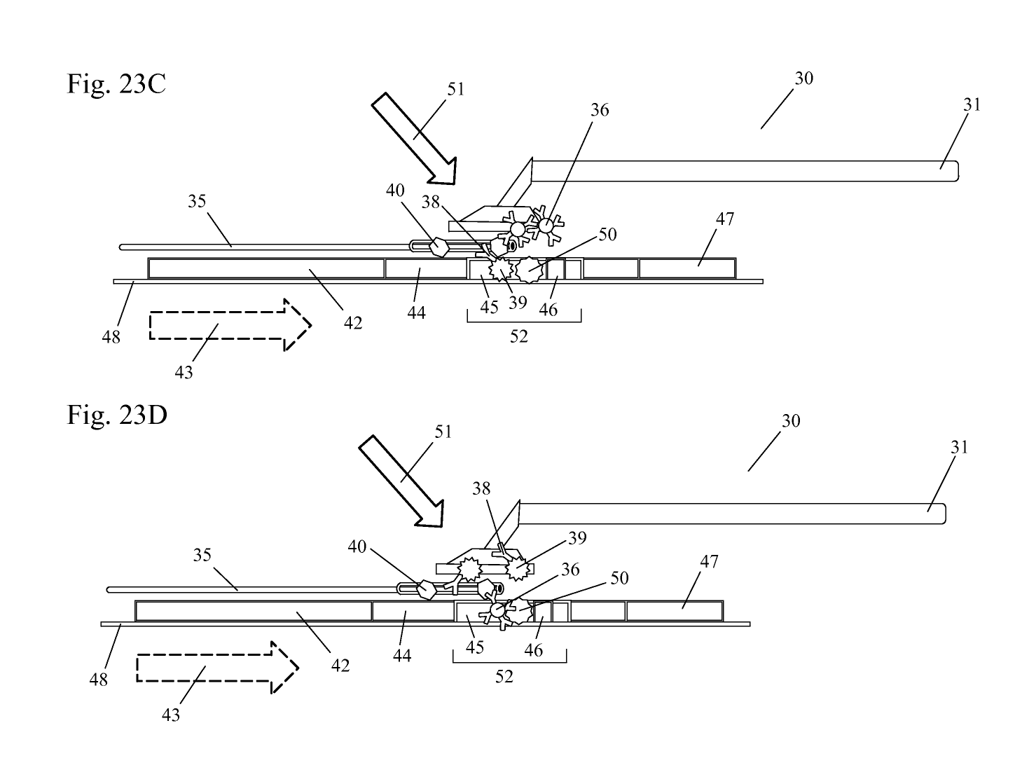

FIGS. 23A through 23D are similar to FIGS. 18C, 19C, 21B, and 22B, respectively, except that the detection zone 52 overlaps the sample application zone 44 in these figures. The detection zone in these embodiments is preferably made of nitrocellulose. Although no lateral flow is strictly required to run the assay in these embodiments, at least a nominal amount of flow is preferred such that the sandwich is able to bind in the test zone and any unbound conjugate is washed out of the test zone. In one embodiment, instead of a running buffer being applied to an end of the test strip, a washing fluid may be applied directly to the test zone, either from above or from the side, for example using a water bottle. In one embodiment, the sample compressor and the sample collector are substantially transparent so that the test zone can be read without removal of the vertical stack from the test strip. Note that, while both the test zone 45 and the control 46 are shown within the sample application zone in these figures, in other embodiments the test zone 45 could overlap the sample application zone 44 while the control zone 46 is downstream of the sample application zone 44. If the control zone was laterally downstream from the sample application zone 44, it would be necessary to add buffer to allow flow. In addition, it may be preferable to add a buffer, for example a buffer that includes silver, to enhance the signal from a positive result.

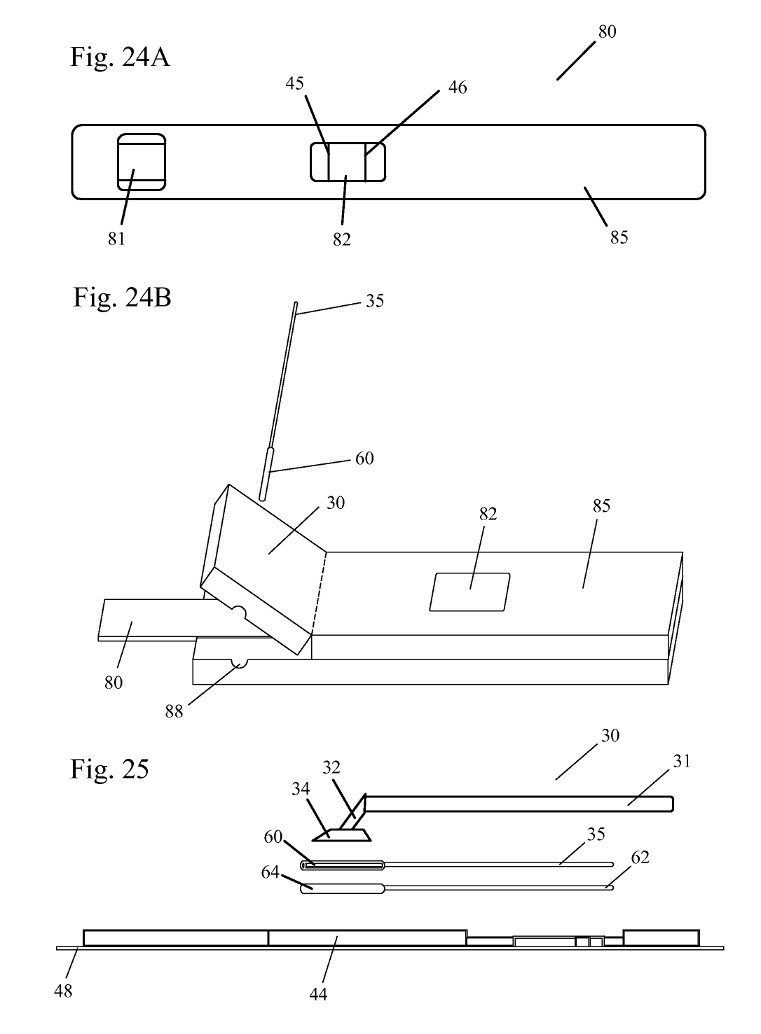

A universal test strip 80, as shown in FIG. 24A, may be used when the sample compressor 30 includes both of the binding partners 37, 38 for the analyte 40. The materials on the sample compressor 30 and the sample collector 35 are transferred to the universal test strip 80 at the sample window 81. Since the elements specific to the analyte 40 being tested are on the sample compressor 30, the test zone 45 in the viewing window 82 of the universal test strip 80 only needs to have a tag 50 that complexes with the tag 39 on the second binding partner 38 for the analyte 40. For example, when the second binding partner 38 for the analyte 40 is tagged 39 with biotin, the test zone 45 of the universal test strip 80 would include avidin 39, a binding partner for biotin. The universal test strip 80 also preferably includes a control zone 46 and a housing 85. For the embodiments of FIGS. 23A through 23D, the test zone is located in the sample window 81.

Although the sample compressor and the sample collector are shown as separate entities in FIGS. 16-24A, the pad 33 of the sample compressor and the sample collector portion 60 of the sample collector may be components of a single element within the spirit of the present invention. For example, the sample collector may be rotatably or flexibly or connected as part of a cartridge to the sample compressor, such that a sample can be collected from a patient with the sample collection portion without exposing the patient to the sample compressor pad and then the sample collection portion and sample compressor pad can be brought into contact for application to the sample application zone of the test strip by compression. The sample collector also may be rotatably or flexibly connected to the test cassette or may be inserted as a cartridge. In another embodiment, the sample may be forcibly injected directly onto the test strip prior to placing the compressor and/or conjugates into position. In yet another embodiment, the sample collector may contact the conjugates in an external cartridge that then snaps or inserts into a test cassette to bring the material in contact with the test strip.

In some embodiments, the sample compressor 30 is rotatably connected to the housing 85 as shown in FIG. 24B. While the hinge of the sample compressor 30 is shown such that the sample compressor 30 is rotated towards the downstream end of the strip when open, the housing could be designed such that the sample compressor 30 is hinged to either side or in other directions within the spirit of the present invention. The sample collection portion 60 of the sample collector 35 is preferably inserted from the side such that it lines up with an insertion hole 88 on the side of the housing 85. However, the sample collector 35 could be inserted in any direction depending upon the design of the housing. The sample compressor 30 preferably includes a pad (not visible in FIG. 24B), with one or more assay components, located on the surface of the sample compressor facing the sample application zone of the test strip 80. The sample compressor 30 is then closed such that a compression pressure is applied to the vertical stack of the pad of the sample compressor, the sample collection portion, and the sample application zone to transfer the sample and the one or more assay components to the sample application zone of the test strip. While there is an absorbent pad sticking out of the housing at the far upstream end of the device in FIG. 24B, the length of the absorbent pad may vary. In fact, as long as buffer can be added at the upstream end (for example, through an application window in the housing), it is not necessary to have the absorbent pad extend significantly outside the housing. In this embodiment, there is no possibility of losing the sample compressor, and there is no need to align the sample compressor with the sample application zone when forming the vertical stack. One advantage of these embodiments is that they allow for a time lapse between sample application and the actual initiation of flow to the test zone. In other words, the sandwich can be pre-made, and the flow initiated much later.

Alternatively, the pad 33 may be separate from the sample compressor within the spirit of the present invention. The pad may be on a binding partner applicator similar to the sample collector. In these embodiments, the binding partner applicator may be located between the sample collection portion and the sample application zone when the pressure is applied by the sample compressor to transfer the sample to the sample application zone.

FIG. 25 shows a vertical stack including a sample compressor 30, a sample collector 35 with a sample collection portion 60, a binding partner applicator 62 with an applicator pad 64, and a sample application zone 44 of a test strip. While the binding partner applicator 62 includes a handle in FIG. 25, the binding partner applicator 62 could alternatively simply be a pad. The ledge portion 34 of the sample compressor 30 applies pressure to the sample collection portion 60 loaded with a sample and the applicator pad 64 loaded with at least one binding partner for an analyte to be tested for in the sample. The pressure preferably forces at least a portion of the sample from the sample collection portion 60 to wet the applicator pad 64, thereby mobilizing some of the binding partner such that at least some of the sample and some of the binding partner are transferred to the sample application zone 44. In some embodiments, this transfer occurs without dilution. In embodiments with small sample volumes or viscous or solid samples, however, an additional liquid may be used to facilitate transfer of the sample and the binding partner to the test strip. In some embodiments, as shown in FIG. 25, the sample compressor has no pad, although a pad may be used to aid in transfer, such as by supplying additional liquid or buffer, within the spirit of the present invention. In some embodiments, as shown in FIG. 25, the sample collection portion 60 is located between the sample compressor 30 and the applicator pad 64 in the vertical stack to aid in transfer of the binding partner to the test strip during compression. Alternatively, the applicator pad 64 may be placed between the sample compressor 30 and the sample collection portion 60 within the spirit of the present invention. In embodiments where the full sandwich forms prior to reaching the test zone, two binding partner applicators (a separate applicator for each binding partner of the analyte) may be used, with the sample collection portion, the first applicator pad, and the second applicator pad being placed in any order on the vertical stack within the spirit of the present invention. Alternatively, a single binding partner applicator could include both of the binding partners for the analyte. In other embodiments, the sample, the first binding partner, and the second binding partner may be applied sequentially to the test strip in any order using the sample compressor within the spirit of the present invention.

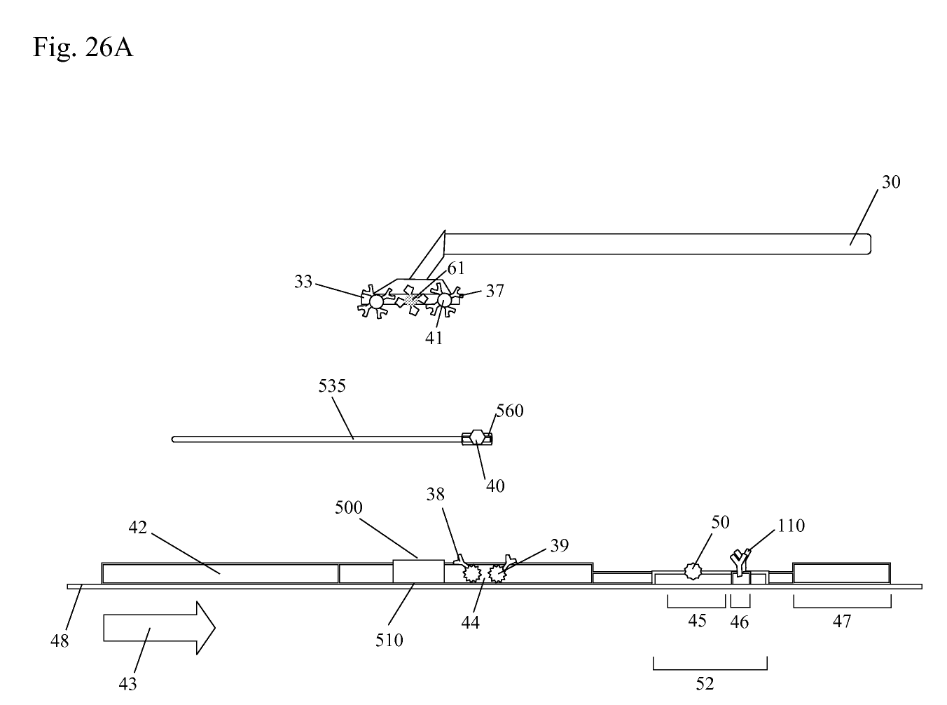

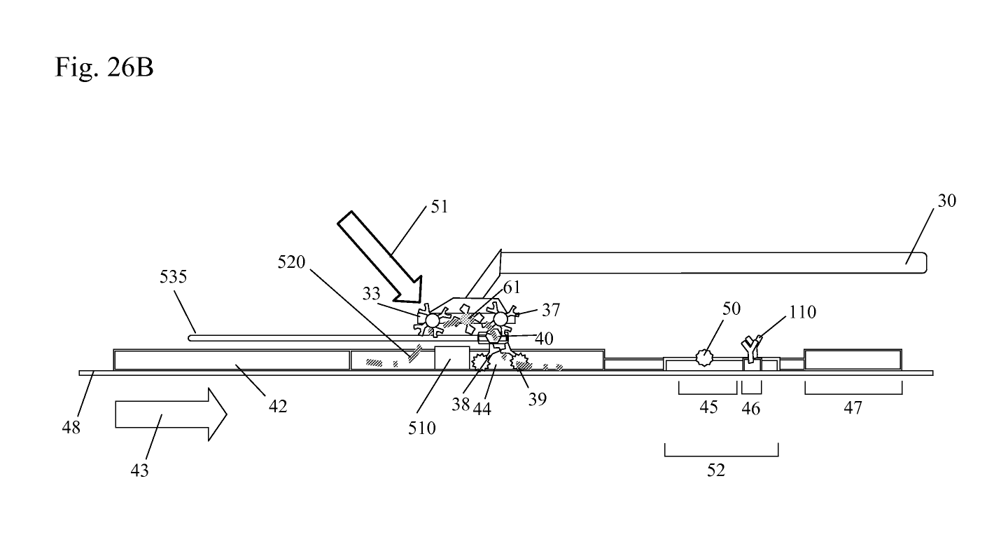

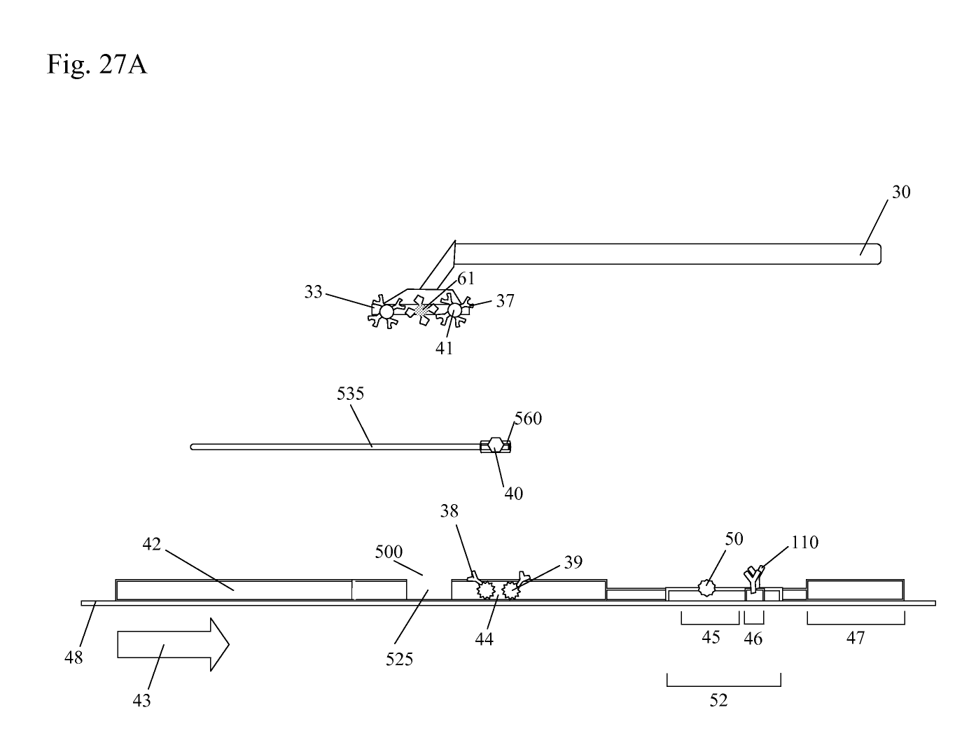

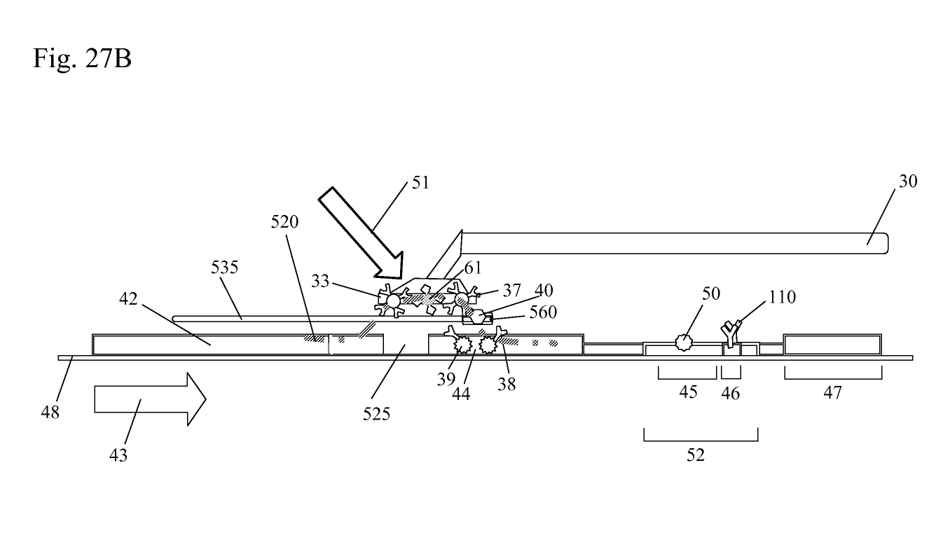

FIGS. 26 and 27 show embodiments of lateral flow devices with a diverting zone 500 in preferred embodiments of the present invention.

FIGS. 26A and 26B show an embodiment with a diverting zone 500 that includes a barrier 510. The system includes a sample compressor 30, a sample collector 535, and a sample analysis device (a test strip in the figure). The test strip preferably includes an absorbent pad 42, a diverting zone 500, a sample application zone 44, a detection zone 52, and an optional waste pad 47. The test strip also preferably includes a carrier backing 48. As shown in FIGS. 26A and 26B, in some embodiments, the collecting portion 560 of the sample collector 535 is compact, so that it concentrates the sample on the collector 560. The diverting zone 500 includes a barrier 510. In other embodiments, the sample collectors 35 shown in previous embodiments could be used. The barrier 510 is preferably an impermeable membrane (or substantially impermeable membrane) that may be made of any material that prevents the flow of liquid from continuing to flow in the same plane. Some materials for the barrier include, but are not limited to, plastics, hydrocarbons or metal.

In this embodiment, the entire sandwich (first binding partner 37—analyte 40—second binding partner 38) forms in the sample application zone 44. The test zone 45 in this embodiment includes an immobilized tag 50 that binds to the tag 39 of the second binding partner 38. In this embodiment, a first binding partner 37, which is part of the conjugate 36 and is preferably pre-loaded and dried on the pad 33 of the sample compressor 30, binds the analyte 40 in the test sample to form a half sandwich. The second binding partner 38 in this embodiment is preferably pre-loaded and dried on the sample application zone 44 of the test strip. The second binding partner 38 also includes a tag 39. Alternatively, the second binding partner 38 in this embodiment may be located anywhere on the test strip upstream of the detection zone including, but not limited to, overlapping the sample application zone, upstream of the sample application zone, and between the sample application zone and the detection zone. Similarly, the sample application zone 44 may be upstream of the diverting zone 500, downstream of the diverting zone 500, or overlapping or on top of the diverting zone 500.

In preferred embodiments, the pad 33 on the sample compressor 30 also includes a control zone binding partner 61 with a detectable label. The control zone binding partner 61 complexes with its binding partner 110 in the control zone 46 when the test has run correctly.

The diverting zone 500 completely stops flow until the sample compressor 30 is brought into contact with the rest of the device, and creates a bridge along which the fluid can flow, as shown by the dotted line 520 in FIG. 26B. The sample compressor 30 acts as a bridge and redirects flow into a different plane. Flow is diverted into the sample compressor 30. This increases collection of the first binding partner 37 and the control zone binding partner 61 on the sample compressor 30. Flow shifts back to the original lateral plane at the end of the diverting zone 500.

In other embodiments, the control zone binding partner 61 could be located on the test strip, for example upstream of the sample application zone, on the sample application zone, or downstream of the sample application zone. In any of the embodiments with a control zone binding partner 61, the control zone binding partner 61 will not reach the control zone 46 unless the sample compressor 30 has effectively formed the bridge, allowing flow to continue past the barrier 510 (as it travels through the sample compressor 30 in an alternate plane) and then back onto the test strip.

In operation, the sample collector 535 is placed such that the sample is directly above the sample application zone 44. The barrier 510 in the diverting zone 500 stops lateral flow 43 on the test strip. When the sample compressor 30 is added, it exerts pressure 51 on the sample collector 535, and creates a bridge over the barrier 510. Flow is diverted 520 into the sample compressor 30 in a separate plane. When the elution medium, sample or buffer flows through the sample compressor 30, it collects the first analyte binding partner 37 of the conjugate 36 and the control zone binding partner 61. Flow travels through the collecting portion 560 of the sample collector 535 as it returns to the test strip after the end of the barrier 510, where the components traveling in the flow interact with the sample of interest. If the analyte 40 is present in the sample, the analyte 40 binds to the first analyte binding partner 36 and the second binding partner 38, creating a “vertical” sandwich with the conjugate 36 as the top piece and the second binding partner 38 as the bottom piece, with the analyte 40 in between them (see FIG. 19B). If there is also a control zone binding partner 61 on the sample compressor 30, the control zone binding partner 61 is also transferred. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

Note that, while FIGS. 26A and 26B show the reagents in a certain configuration, the reagents may be placed in alternative configurations, for example the configurations shown in FIGS. 16, 18, 19, 22, 23 and 24 with the addition of the barrier shown in FIG. 26.

Although the barrier is shown as a specific length relative to the rest of the test strip in FIGS. 26A and 26B, the figures are schematic. The barrier 510 may be of any length on the test strip sufficient to stop flow and require the sample compressor 30 to recommence flow. The barrier 510 is designed to not be so long as to obstruct flow back into the lateral plane at the downstream end of the sample compressor 30.

In one preferred embodiment, the barrier 510 includes encapsulated components. The barrier 510 in these embodiments is made of a material that dissolves over time (as disclosed in US Patent Publication No. 2013/0017559, published Jan. 17, 2013, herein incorporated by reference), releasing the encapsulated components. The barrier 510 may include any or all of the same reagents that have been discussed herein as being able to be encapsulated. A dissolving barrier 510 performs dual functions. Similar to the other barriers 510, it acts as a wall to force flow into the sample compressor. In addition, it time delays certain components by encapsulating them. Buffer or elution medium slowly dissolves the barrier 510, and these time-delayed components will impact the test line complex after the other components of the assay have reached the test line.

FIGS. 27A and 27B differ from FIGS. 26A and 26B in that the barrier 510 of the diverting zone 500 is replaced with a gap or ditch 525. The gap 525 interrupts flow by removing the membranes that permit flow along the test strip.

The diverting zone 500 completely stops flow until the sample compressor 30 is brought into contact with the rest of the device, and creates a bridge along which the fluid can flow, as shown by the dotted line 520 in FIG. 27B. The sample compressor 30 acts as a bridge and redirects flow into a different plane. Flow is diverted into the sample compressor 30. This increases collection of the first binding partner 37 and the control zone binding partner 61 on the sample compressor 30. Flow shifts back to the original lateral plane at the end of the diverting zone 500.

In other embodiments, the control zone binding partner 61 could be located on the test strip, for example upstream of the sample application zone 44, on the sample application zone 44, or downstream of the sample application zone 44. In any of the embodiments with a control zone binding partner 61, the control zone binding partner 61 will not reach the control zone 46 unless the sample compressor 30 has effectively formed the bridge, allowing flow to continue past the gap (as it travels through the sample compressor 30 in an alternate plane) and then back onto the test strip.

In operation, the sample collector 535 is placed such that the sample is directly above the sample application zone 44. The gap 525 in the diverting zone 500 stops lateral flow 43 on the test strip. When the sample compressor 30 is added, it exerts pressure 51 on the sample collector 535, and creates a bridge over the gap 525. Flow is diverted 520 into the sample compressor 30 in a separate plane. When the elution medium, sample or buffer flows through the sample compressor 30, it collects the first analyte binding partner 37 of the conjugate 36 and the control zone binding partner 61. Flow travels through the collecting portion 560 of the sample collector 535 as it returns to the test strip after the end of the gap 525, where the components traveling in the flow interact with the sample of interest. If the analyte 40 is present in the sample, the analyte 40 binds to the first analyte binding partner 36 and the second binding partner 38, creating a “vertical” sandwich with the conjugate 36 as the top piece and the second binding partner 38 as the bottom piece, with the analyte 40 in between them (see FIG. 19B). If there is also a control zone binding partner 61 on the sample compressor 30, the control zone binding partner 61 is also transferred. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

Note that, while FIGS. 27A and 27B show the reagents in a certain configuration (similar to FIGS. 21A through 21B), the reagents may be placed in alternative configurations, for example the configurations shown in FIGS. 16, 18, 19, 22, 24 and 25 with the addition of the gap shown in FIG. 27. In addition, the embodiment shown in FIG. 27 could be used in combination with enhancement elements or encapsulation embodiments disclosed herein.

Although the gap 525 is shown in FIGS. 27A and 27B as extending down to the carrier backing, the gap 525 only needs to be of sufficient depth to stop flow.

In other preferred embodiments, more than one barrier, more than one gap, or a combination of at least one barrier and at least one gap may make up the diverting zone.

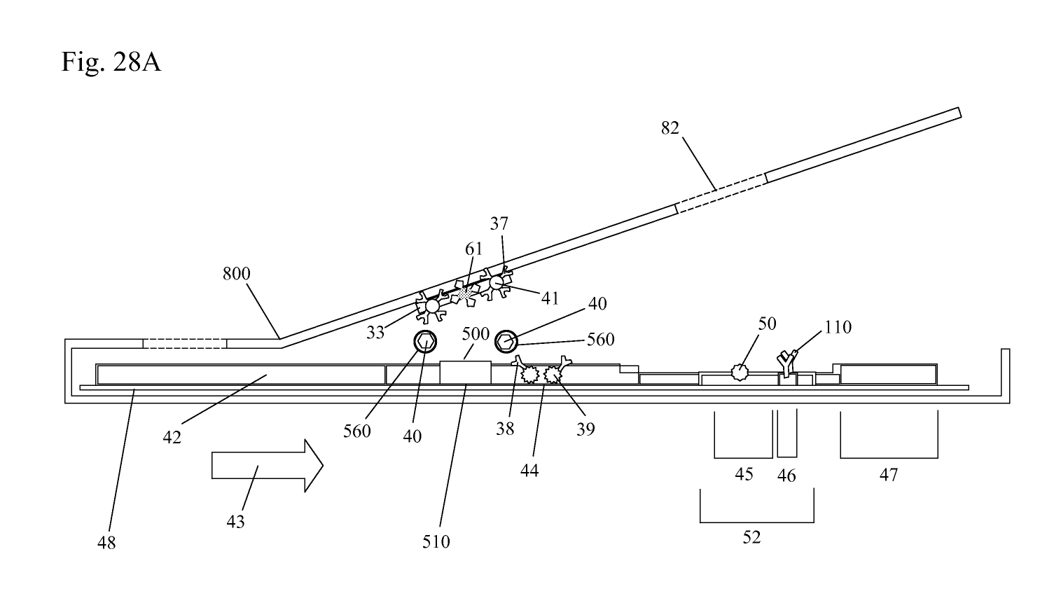

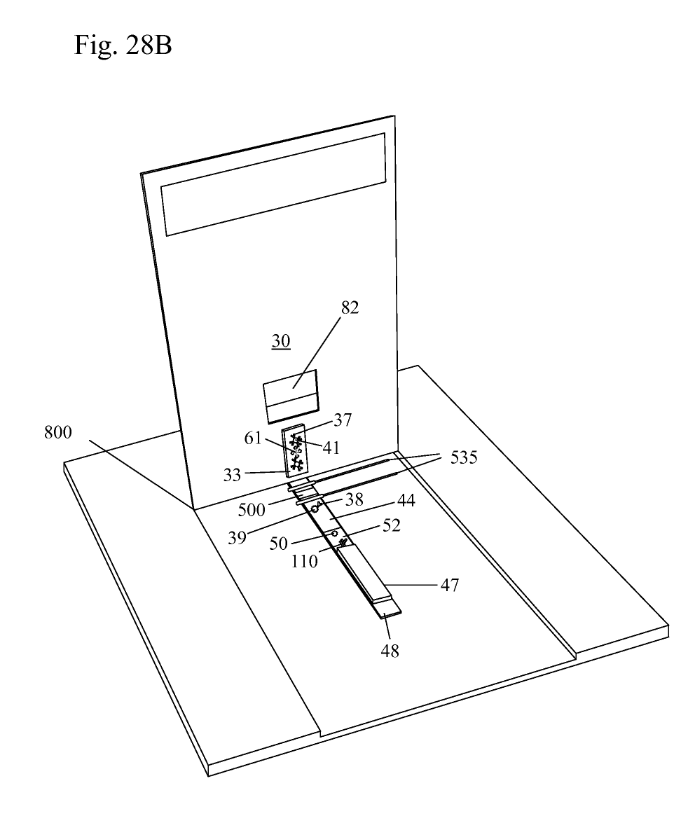

FIGS. 28A and 28B show a lateral flow device with a hinge 800, a diverting zone 500, and a sample compressor 30 in another embodiment of the present invention. The hinge 800 facilitates compression, but this embodiment otherwise functions similarly to the diverting zone embodiments described in FIGS. 26A and 26B. The hinge 800 and the sample compressor pad 33 in this embodiment could be used with any of the embodiments described herein. The hinge configuration in FIG. 24B could alternatively be used with a diverting zone 500 in other embodiments of the invention. Note that, while the sample collector 535 is shown in these figures, the sample collector 35 could alternative be used.

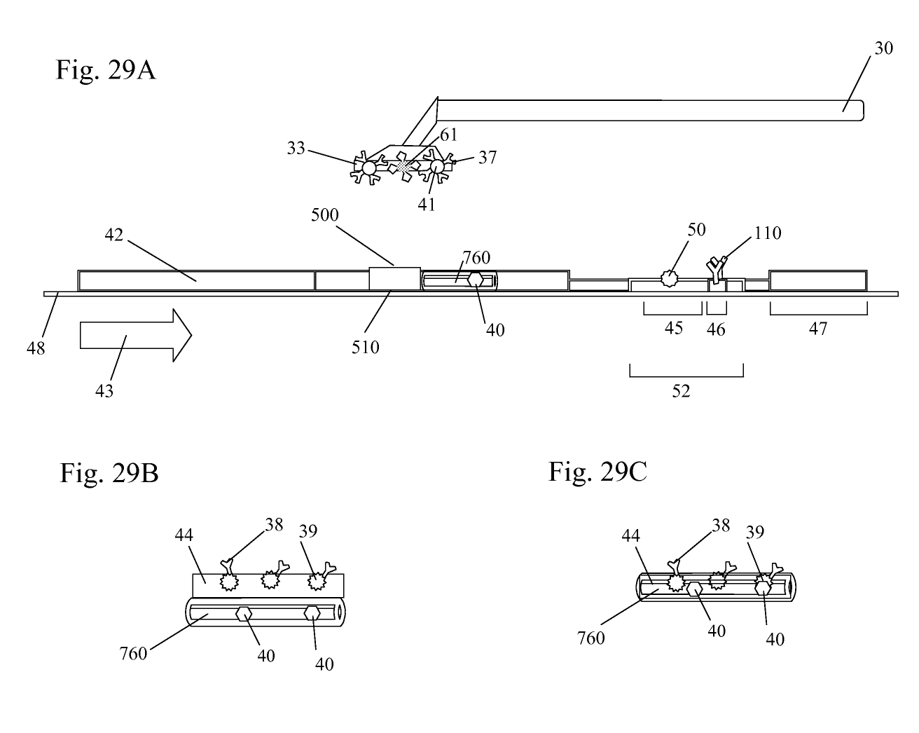

FIGS. 29A-29C show a lateral flow device with a diverting zone 500, a sample compressor 30, and a chromatographic test strip including a separator paper 760 in an embodiment of the present invention. In this embodiment, a least one separator paper 760 is part of the chromatographic test strip. The device shown in FIGS. 29A-29C includes at least one separator paper 760 located adjacent to a sample application zone 44 on the chromatographic test strip. To facilitate application of sample to the separator paper 760, the separator paper is preferably located adjacent the path of lateral flow (in the same plane). FIG. 29A shows a side view of the device, so only the separator paper 760 is visible. FIG. 29B shows a top down view of the sample application zone 44 and the adjacent separator paper 760. A sample is added to the separator paper 760, for example a liquid sample added with a pipette or another sample adding device, before running the assay. Prior to compression with the sample compressor 30, the separator paper 760 is flipped onto the sample application zone 44 of the lateral flow path, as shown in FIG. 29C. While the separator paper 760 is shown downstream of the bather 510 in FIG. 29A, the separator paper 760 and the sample application zone 44 may optionally be upstream of the barrier 510, or even on or overlapping the barrier 510 in alternative embodiments. If there are multiple separator papers 760, they may be located in different places on the device. The function and structure of the device is otherwise similar to the device shown and described in FIGS. 27A and 27B.

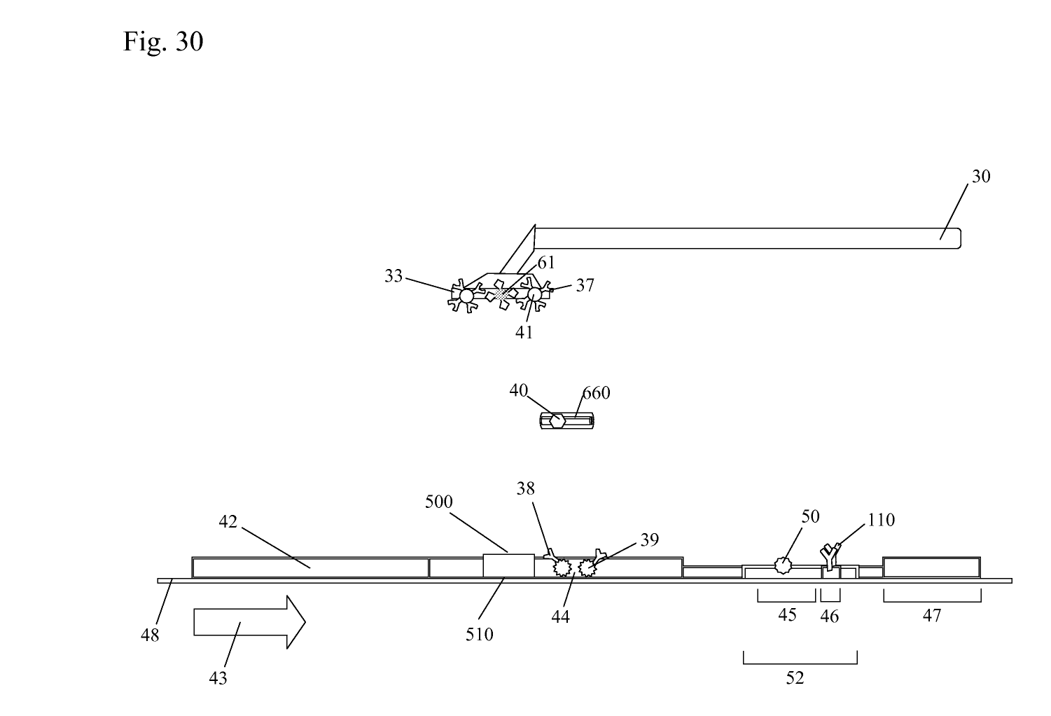

While FIGS. 26-27 show a swab member 35, 535 with a sample collecting portion 60, 560, in other embodiments, the sample collection device is at least one separator paper 660 that is placed in the same location in the vertical stack as the sample collection portion 60, 560 of the swab member 35, 535 shown in the figures. As an example, the device shown in FIG. 30 replaces the swab member 35, 535 with a separator paper 660. While the separator paper 660 and sample application zone 44 are shown downstream of the bather 510 in FIG. 30, the separator paper 660 may optionally be applied upstream of the barrier 510, or even on or overlapping the bather 510 in alternative embodiments. In other embodiments, multiple separator papers may be used and located in different locations on the device. This device in this figure otherwise operates similarly to the device described and shown in FIGS. 26A and 26B.

One or more separator papers 660 or 760 may be used as the sample collector in any of the embodiments described herein.

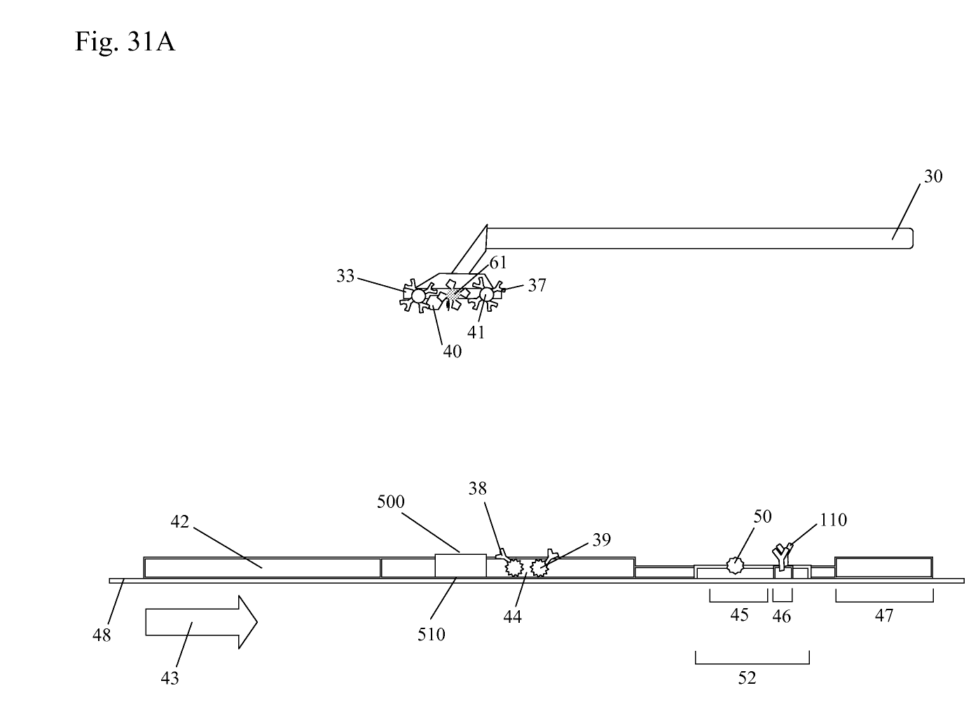

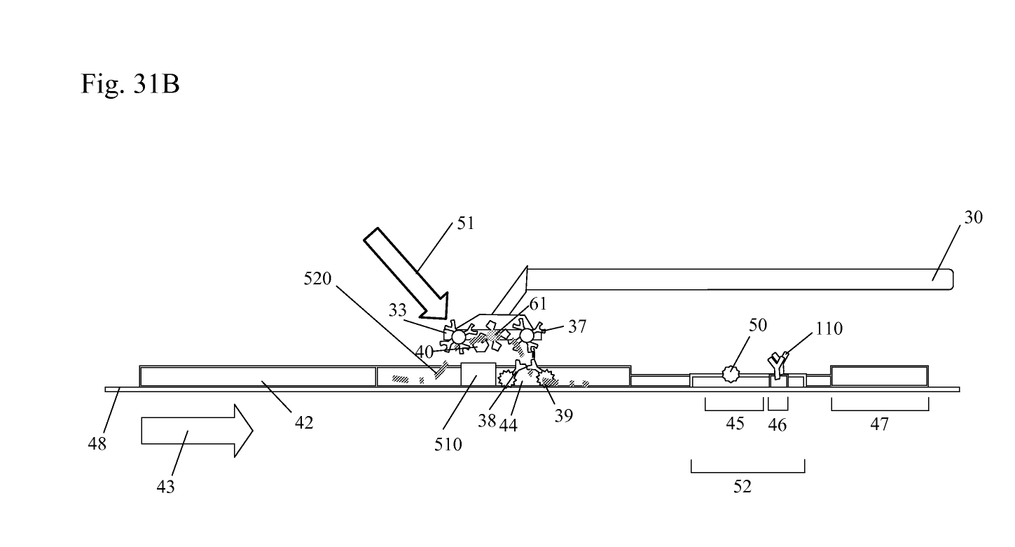

FIGS. 31A and 31B show another embodiment with a diverting zone 500 that includes a bather 510 similar to that of FIGS. 26A and 26B. The embodiment of FIGS. 31A and 31B differs from the embodiment of FIGS. 26A and 26B in that a separate sample collector is not used. Instead, the sample is directly added to the sample compressor 30. An analyte 40 is shown on the sample compressor 30 to depict that the sample has been added to the sample compressor 30. While the sample application zone 44 in this embodiment is the location where the sample first encounters the test strip, the sample in this embodiment is added to the sample compressor 30 and travels in the running buffer to the sample application zone 44 of the test strip.

In this embodiment, ½ of the sandwich (the first binding partner 37—analyte 40) begins to form on the sample compressor 30, and the entire sandwich (first binding partner 37—analyte 40—second binding partner 38) forms before the sample reaches the test zone 45. The second binding partner 38 in this embodiment is preferably pre-loaded and dried on the sample application zone 44 of the test strip. Alternatively, the second binding partner 38 in this embodiment may be located anywhere on the test strip upstream of the detection zone including, but not limited to, overlapping the sample application zone, upstream of the sample application zone, and between the sample application zone and the detection zone.

In operation, the sample is placed on the sample compressor 30. The barrier 510 in the diverting zone 500 stops lateral flow 43 on the test strip. When the sample compressor 30 is added, it creates a bridge over the barrier 510. Flow is diverted 520 into the sample compressor 30 in a separate plane. When the elution medium, sample or buffer flows through the sample compressor 30, it collects the sample, the first analyte binding partner 37 of the conjugate 36 and the control zone binding partner 61. Flow returns to the test strip after the end of the barrier 510. If the analyte 40 is present in the sample, the analyte 40 binds to the first analyte binding partner 36 and the second binding partner 38, creating a “vertical” sandwich with the conjugate 36 as the top piece and the second binding partner 38 as the bottom piece, with the analyte 40 in between them. If there is also a control zone binding partner 61 on the sample compressor 30, the control zone binding partner 61 is also transferred. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

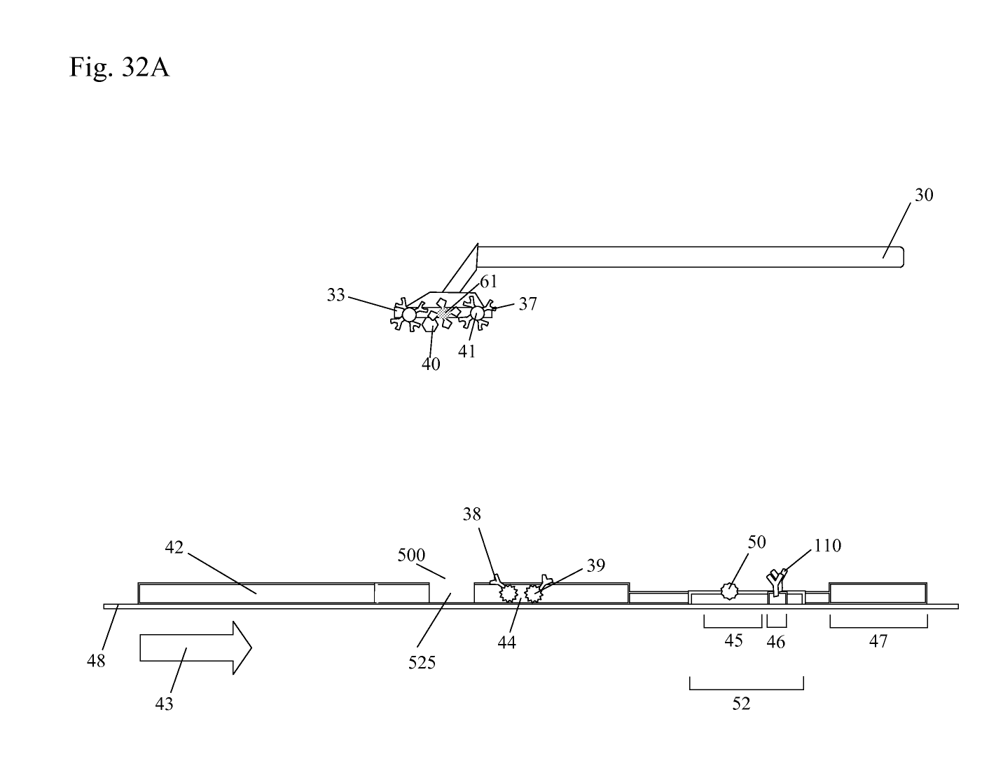

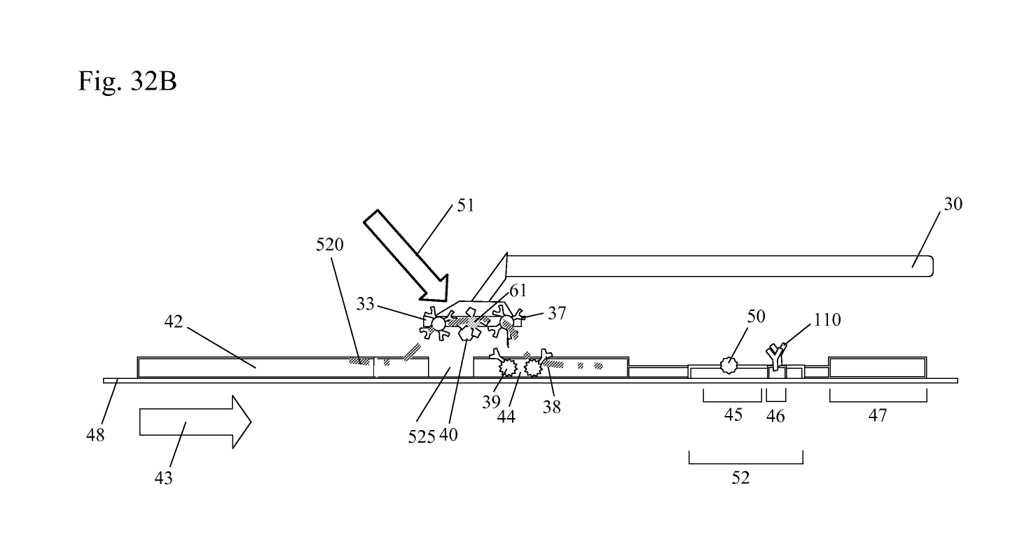

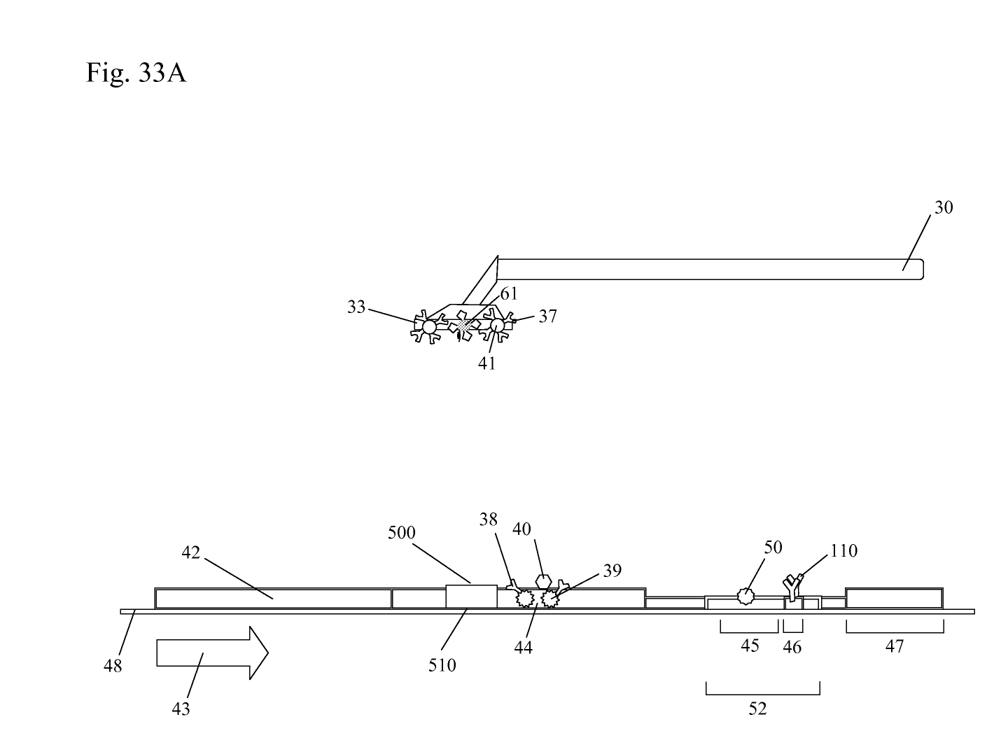

FIGS. 32A and 32B show a diverting zone 500 with a gap or ditch 525 similar to that of FIGS. 27A and 27B. The embodiment of FIGS. 32A and 32B differs from the embodiment of FIGS. 27A and 27B in that a separate sample collector is not used. In this embodiment, the sample is directly added to the sample compressor 30. An analyte 40 is shown on the sample compressor 30 to depict that the sample has been added to the sample compressor 30. While the sample application zone 44 in this embodiment is the location where the sample first encounters the test strip, the sample in this embodiment is added to the sample compressor 30 and travels in the running buffer to the sample application zone 44 of the test strip.

In this embodiment, ½ of the sandwich (the first binding partner 37—analyte 40) begins to form on the sample compressor 30, and the entire sandwich (first binding partner 37—analyte 40—second binding partner 38) forms before the sample reaches the test zone 45. In this embodiment, a first binding partner 37, which is part of the conjugate 36 and is preferably pre-loaded and dried on the pad 33 of the sample compressor 30, binds the analyte 40 in the test sample to form a half sandwich. The second binding partner 38 in this embodiment is preferably pre-loaded and dried on the sample application zone 44 of the test strip. The second binding partner 38 also includes a tag 39. Alternatively, the second binding partner 38 in this embodiment may be located anywhere on the test strip upstream of the detection zone including, but not limited to, overlapping the sample application zone, upstream of the sample application zone, and between the sample application zone and the detection zone.

In operation, the gap 525 in the diverting zone 500 stops lateral flow 43 on the test strip. When the sample compressor 30 is added, it creates a bridge over the gap 525. Flow is diverted 520 into the sample compressor 30 in a separate plane. When the elution medium, sample or buffer flows through the sample compressor 30, it collects the sample, the first analyte binding partner 37 of the conjugate 36 and the control zone binding partner 61. Flow returns to the test strip after the end of the gap 525. If the analyte 40 is present in the sample, the analyte 40 binds to the first analyte binding partner 36 and the second binding partner 38, creating a “vertical” sandwich with the conjugate 36 as the top piece and the second binding partner 38 as the bottom piece, with the analyte 40 in between them. If there is also a control zone binding partner 61 on the sample compressor 30, the control zone binding partner 61 is also transferred. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

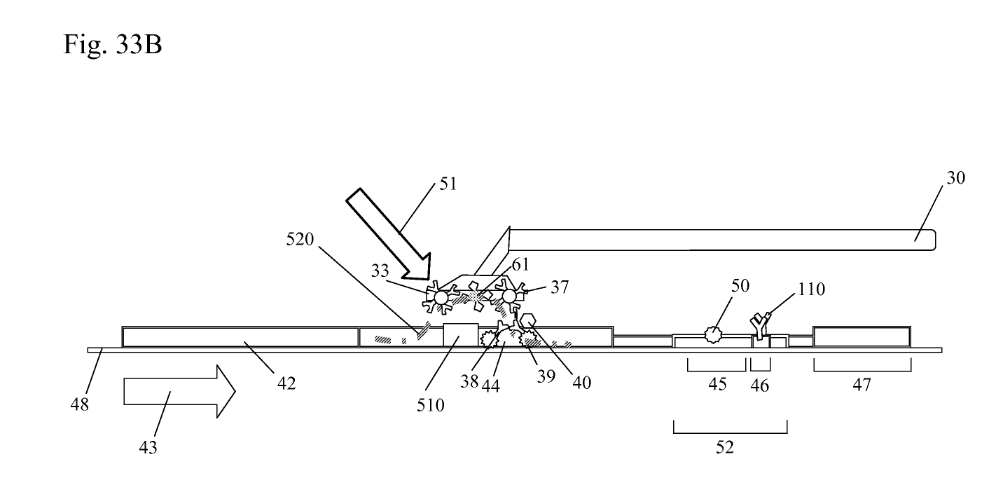

FIGS. 33A and 33B show another embodiment with a diverting zone 500 that includes a barrier 510 similar to that of FIGS. 31A and 31B. The embodiment of FIGS. 33A and 33B differs from the embodiment of FIGS. 31A and 31B in that the sample is applied directly to the sample application zone 44 instead of the pad 33 of the sample compressor 30. An analyte 40 is shown on the sample application zone 44 to depict that the sample has been added to the sample application zone 44.

The entire sandwich (first binding partner 37—analyte 40—second binding partner 38) forms in the sample application zone 44. In this embodiment, a first binding partner 37, which is part of the conjugate 36 and is preferably pre-loaded and dried on the pad 33 of the sample compressor 30, binds the analyte 40 in the test sample to form a half sandwich. The second binding partner 38 in this embodiment is preferably pre-loaded and dried on the sample application zone 44 of the test strip. The second binding partner 38 also includes a tag 39. Alternatively, the second binding partner 38 in this embodiment may be located anywhere on the test strip upstream of the detection zone including, but not limited to, overlapping the sample application zone, upstream of the sample application zone, and between the sample application zone and the detection zone. Similarly, the sample application zone 44 may be upstream of the diverting zone 500, downstream of the diverting zone 500, or overlapping or on top of the diverting zone 500.

In operation, the sample is placed on the sample application zone 44. The barrier 510 in the diverting zone 500 stops lateral flow 43 on the test strip. When the sample compressor 30 is added, it exerts pressure 51 on the test strip, and creates a bridge over the barrier 510. Flow is diverted 520 into the sample compressor 30 in a separate plane. When the elution medium, sample or buffer flows through the sample compressor 30, it collects the first analyte binding partner 37 of the conjugate 36 and the control zone binding partner 61. Flow returns to the test strip after the end of the barrier 510, where the components traveling in the flow interact with the sample of interest. If the analyte 40 is present in the sample, the analyte 40 binds to the first analyte binding partner 36 and the second binding partner 38, creating a “vertical” sandwich with the conjugate 36 as the top piece and the second binding partner 38 as the bottom piece, with the analyte 40 in between them. If there is also a control zone binding partner 61 on the sample compressor 30, the control zone binding partner 61 is also transferred. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

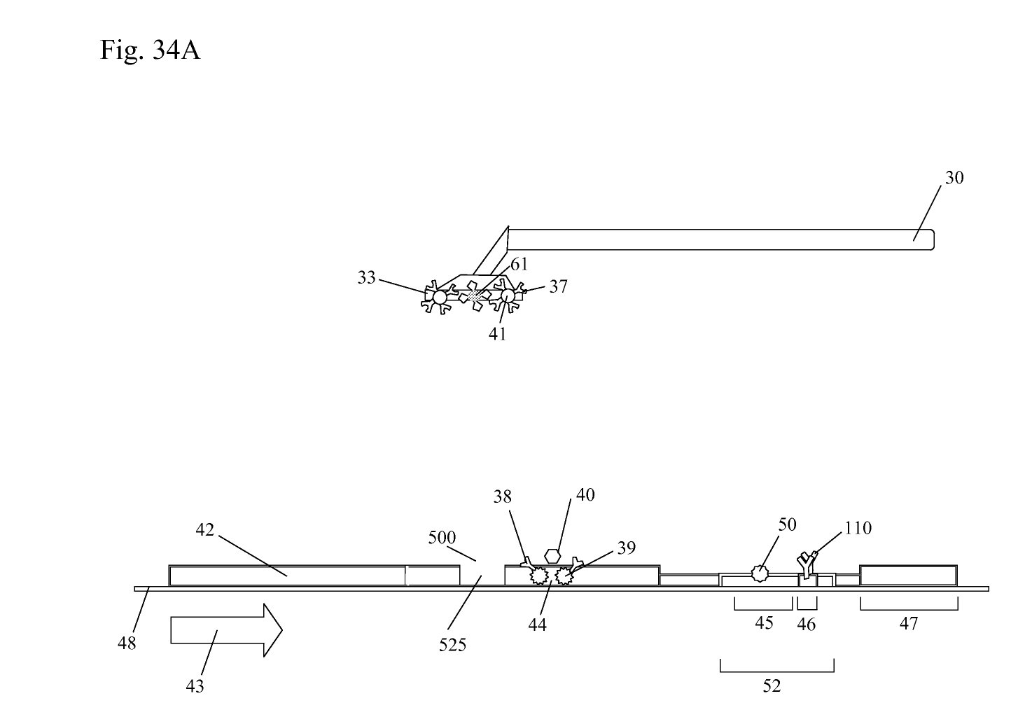

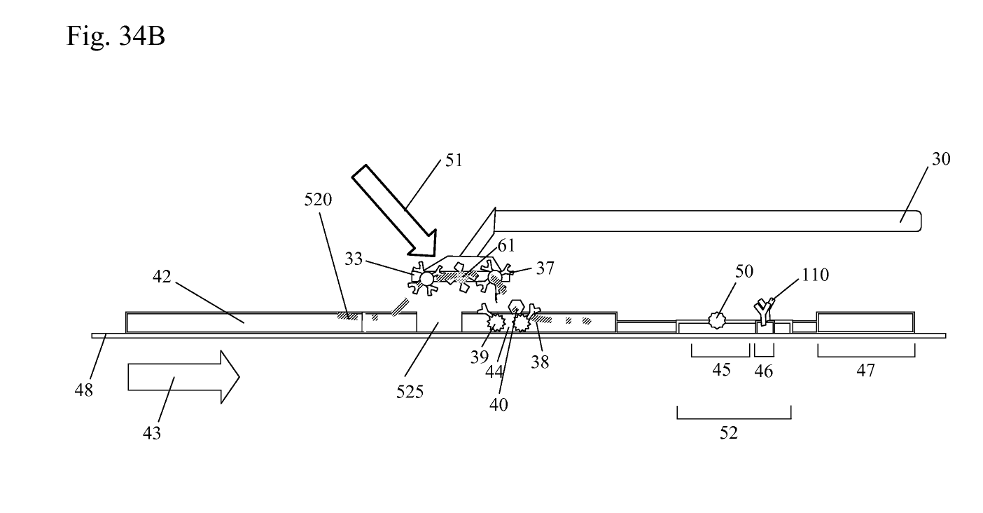

FIGS. 34A and 34B show a diverting zone 500 with a gap or ditch 525 similar to that of FIGS. 32A and 32B. The embodiment of FIGS. 34A and 34B differs from the embodiment of FIGS. 32A and 32B in that the sample is applied directly to the sample application zone 44 instead of the pad 33 of the sample compressor 30. An analyte 40 is shown on the sample application zone 44 to depict that the sample has been added to the sample application zone 44.

The entire sandwich (first binding partner 37—analyte 40—second binding partner 38) forms in the sample application zone 44. In this embodiment, a first binding partner 37, which is part of the conjugate 36 and is preferably pre-loaded and dried on the pad 33 of the sample compressor 30, binds the analyte 40 in the test sample to form a half sandwich. The second binding partner 38 in this embodiment is preferably pre-loaded and dried on the sample application zone 44 of the test strip. The second binding partner 38 also includes a tag 39. Alternatively, the second binding partner 38 in this embodiment may be located anywhere on the test strip upstream of the detection zone including, but not limited to, overlapping the sample application zone, upstream of the sample application zone, and between the sample application zone and the detection zone. Similarly, the sample application zone 44 may be upstream of the diverting zone 500, downstream of the diverting zone 500, or overlapping or on top of the diverting zone 500.

In operation, the gap 525 in the diverting zone 500 stops lateral flow 43 on the test strip. When the sample compressor 30 is added, it creates a bridge over the gap 525. Flow is diverted 520 into the sample compressor 30 in a separate plane. When the elution medium, or buffer flows through the sample compressor 30, it collects the first analyte binding partner 37 of the conjugate 36 and the control zone binding partner 61. Flow returns to the test strip after the end of the gap 525, where the components traveling in the flow interact with the sample of interest. If the analyte 40 is present in the sample, the analyte 40 binds to the first analyte binding partner 36 and the second binding partner 38, creating a “vertical” sandwich with the conjugate 36 as the top piece and the second binding partner 38 as the bottom piece, with the analyte 40 in between them (see FIG. 19B). If there is also a control zone binding partner 61 on the sample compressor 30, the control zone binding partner 61 is also transferred. An immobilized tag 50 in the test zone 45 then binds the tag 39. Since the conjugate 36 includes a label 41, the complex that forms is detectable and indicates a positive result. Proper operation of the test also results in a detectable positive result in the control zone 46 due to the interaction between the control zone binding partner 61 and its immobilized partner in the control zone 46.

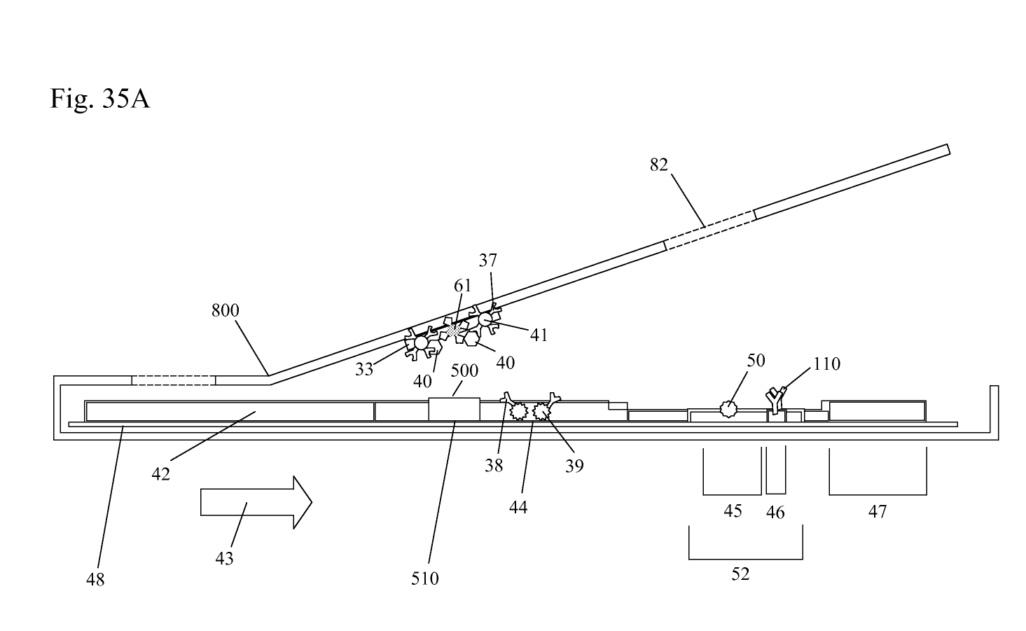

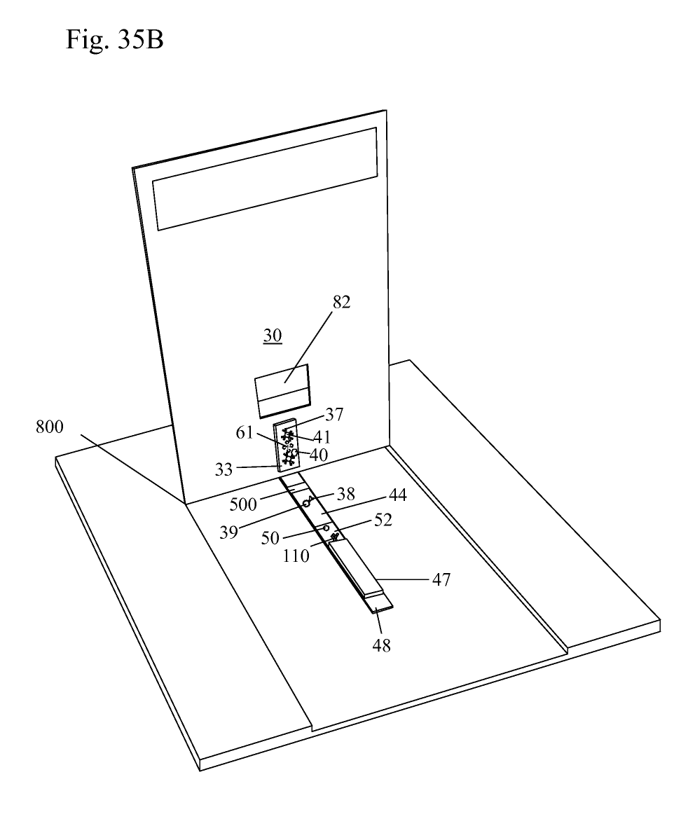

FIGS. 35A and 35B show a lateral flow device with a hinge 800, a diverting zone 500, and a sample compressor 30 in another embodiment of the present invention. The hinge 800 facilitates compression, but this embodiment otherwise functions similarly to the diverting zone embodiments described in FIGS. 29 and 30. The hinge 800 and the sample compressor pad 33 in this embodiment could be used with any of the embodiments described herein. The hinge configuration in FIG. 24B could alternatively be used with a diverting zone 500 in other embodiments of the invention.

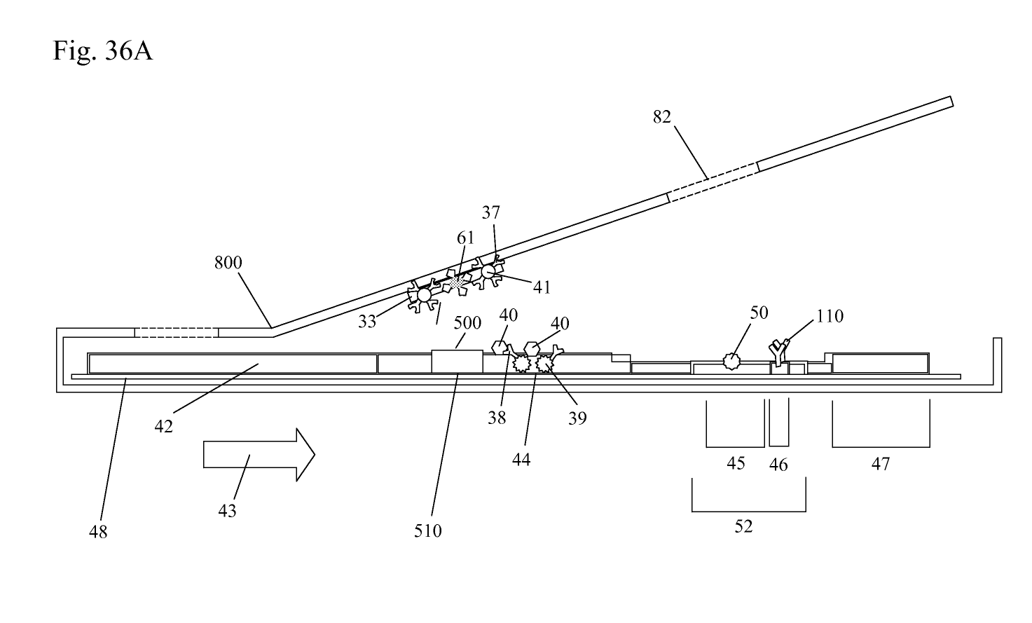

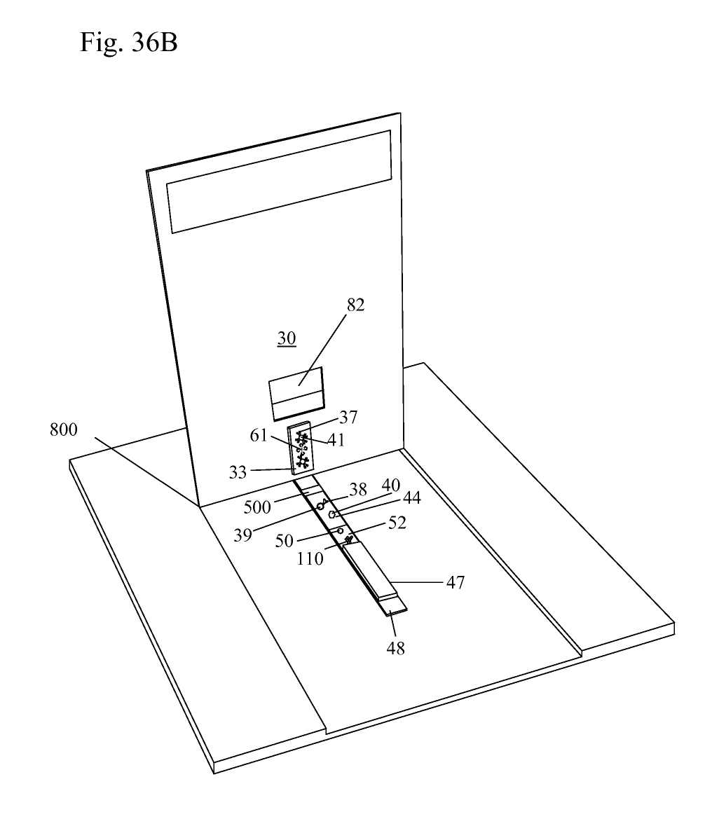

FIGS. 36A and 36B show a lateral flow device with a hinge 800, a diverting zone 500, and a sample compressor 30 in another embodiment of the present invention. The hinge 800 facilitates compression, but this embodiment otherwise functions similarly to the diverting zone embodiments described in FIGS. 31 and 32. The hinge 800 and the sample compressor pad 33 in this embodiment could be used with any of the embodiments described herein.

Although FIGS. 26-36 are described using binding partners for the analyte upstream of the test zone, with tags 50 immobilized in the test zone, in other alternative embodiments, the second binding partner 38 for the analyte could be immobilized in the test zone in all of the test strip configurations described in FIGS. 26-36. In these embodiments, only ½ of the sandwich (first binding partner 37—analyte 40) forms before the sample reaches the test zone.

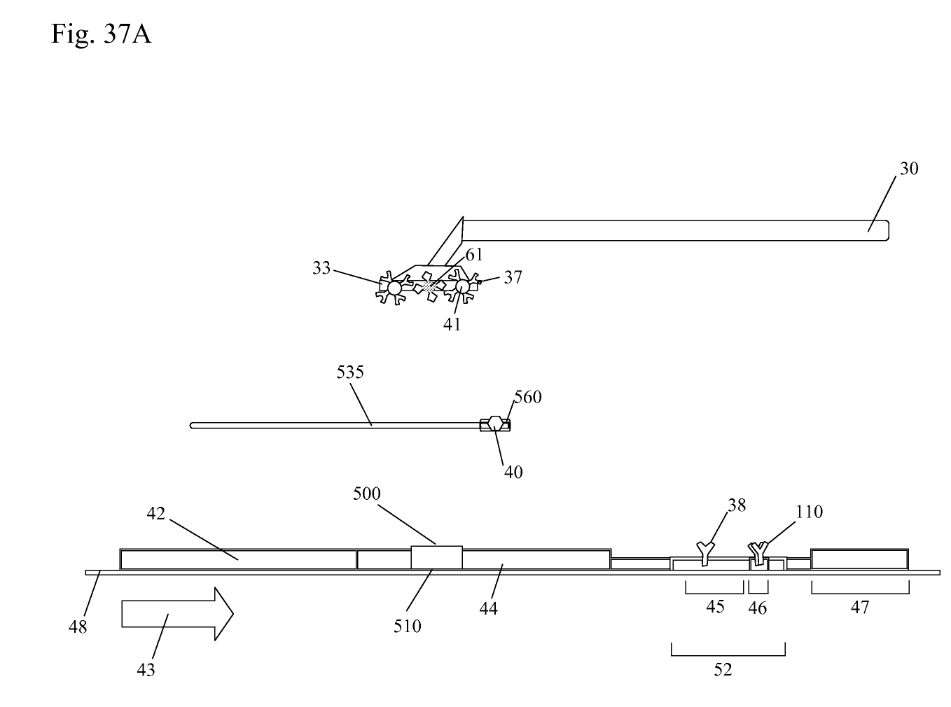

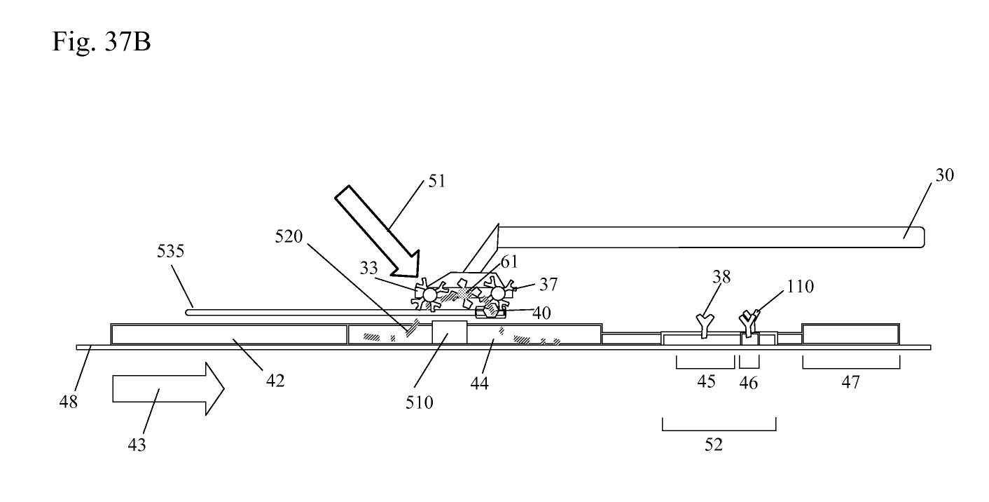

Embodiments with the second binding partner 38 in the test zone 45 are shown in FIGS. 37A and 37B, FIGS. 38A and 38B, FIGS. 39A and 39B, FIG. 40, FIG. 41, FIGS. 42A and 42B, FIGS. 43A and 43B, FIGS. 44A and 44B, FIGS. 45A and 45B, FIGS. 46A and 46B, and FIGS. 47A and 47B. These embodiments are similar to the embodiments shown in FIGS. 26A and 26B, FIGS. 27A and 27B, FIGS. 28A and 28B, FIGS. 29A through 29C, FIG. 30, FIG. 31, FIGS. 32A and 32B, FIGS. 33A and 33B, FIGS. 34A and 34B, FIGS. 35A and 35B, and FIGS. 36A and 36B, respectively, except that there is no tag 39 or immobilized tag 50 and the second binding partner 38 is immobilized in the test zone 45. Consequently, the full sandwich (first binding partner 37—analyte 40—second binding partner 38) is not formed until the sample reaches the test zone 45.

In other preferred embodiments, as alternatives to the embodiment of any of FIGS. 26A through 47B, more than one barrier, more than one gap, or a combination of at least one barrier and at least one gap may make up the diverting zone.

In other embodiments with a sample compressor 30, the sample compressor does not include any reagents for the test, and is used only to provide pressure or to bridge a diverting zone on the test strip. In still other embodiments, both the first binding partner and the second binding partner are located on the sample compressor.

In inhibition assays, only a ½ sandwich is formed, but diverting zones, sample compressors, and other components of the assay configurations of FIGS. 9-47 could otherwise be used in those assays.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of detecting a presence of a chemical warfare nerve agent in a sample, comprising the steps of:
a) collecting the sample;
b) transferring the sample to a sample analysis device comprising:
i) at least one conjugate zone comprising a conjugate comprising a mammalian albumin, a label and a first binding partner for an antibody in a blocking antibody zone;
ii) the blocking antibody zone comprising a plurality of antibodies immobilized in the blocking antibody zone, wherein the antibodies bind to the first binding partner and also bind to the chemical warfare nerve agent; and
iii) at least one test zone comprising a protein that binds to mammalian albumin;
wherein, when the analyte is present in the sample, the analyte and the first binding partner compete for a binding site on the antibodies immobilized in the blocking antibody zone and at least a portion of the conjugate reaches the test zone;
wherein, when the chemical warfare nerve agent is not present in the sample, the first binding partner binds to the binding site on the antibodies immobilized in the blocking antibody zone and the conjugate does not reach the test zone; and
c) detecting the presence of the chemical warfare nerve agent in the test zone.

2. The method of claim 1, wherein the protein that binds to mammalian albumin is Albegone.

3. The method of claim 1, wherein the antibodies are selected from the group consisting of ID8.2 and BE2.

4. The method of claim 1, further comprising the step of exposing at least one antibody reagent to Fclock recombinant protein, such that the Fclock recombinant protein binds to an Fc portion of the antibody.

5. A method of detecting a presence of a chemical warfare nerve agent in a sample, comprising the steps of:
a) collecting the sample;
b) transferring the sample to a sample analysis device comprising:
i) at least one conjugate zone comprising a conjugate comprising a first binding partner for the chemical warfare nerve agent and a label;
ii) a hapten zone comprising the chemical warfare nerve agent or an analog to the chemical warfare nerve agent, immobilized in the hapten zone;
iii) at least one test zone comprising a second binding partner for the conjugate that is different than the first binding partner for the chemical warfare nerve agent, immobilized in the test zone;
wherein, when the chemical warfare nerve agent is present in the sample, the chemical warfare nerve agent in the sample and the chemical warfare nerve agent or chemical warfare nerve agent analog in the hapten zone compete for the first binding partner on the conjugate, and at least a portion of the conjugate reaches the test zone;
wherein, when the chemical warfare nerve agent is not present in the sample, the chemical warfare nerve agent or chemical warfare nerve agent analog in the hapten zone bind to the first binding partner on the conjugate and the conjugate does not reach the test zone; and
c) detecting the presence of the chemical warfare nerve agent in the test zone.

6. The method of claim 5, wherein the conjugate is selected from the group consisting of ID8.2 and BE2.

7. The method of claim 5, further comprising the step of exposing at least one antibody reagent to Fclock recombinant protein, such that the Fclock recombinant protein binds to an Fc portion of the antibody.

8. A method of detecting a presence of a chemical warfare nerve agent in a sample, comprising the steps of:
a) collecting the sample;
b) transferring the sample to a sample analysis device comprising:
i) a conjugate zone comprising a conjugate comprising a binding partner for the chemical warfare nerve agent and a label; and
ii) a detection zone comprising a protein that binds to mammalian albumin, located laterally downstream from the conjugate zone; and
c) detecting the presence of the chemical warfare nerve agent in the detection zone.

9. The method of claim 8, wherein the binding partner for the analyte is selected from the group consisting of ID8.2 and BE2.

10. The method of claim 8, wherein the protein that binds to mammalian albumin is Albegone.

11. The method of claim 8, further comprising the step of exposing at least one antibody reagent to Fclock recombinant protein, such that the Fclock recombinant protein binds to an Fc portion of the antibody.

12. A method of detecting an organic analyte in a sample, comprising the steps of:
a) collecting a sample;
b) transferring the sample to a microcapillary tube comprising C18 coated microcapillaries;
c) exposing the sample to at least one conjugate, comprising a binding partner for the organic analyte and a label; and
d) detecting the presence of the organic analyte in the microcapillary tube.

13. The method of claim 12, further comprising the steps of e) washing the microcapillaries to remove unbound materials.

14. The method of claim 12, wherein the sample is a urine sample and the organic analyte is a chemical warfare nerve agent.

15. The method of claim 14, wherein the organic analyte is a chemical warfare nerve agent and the binding partner for the organic analyte is ID8.2 or BE2.

16. A device for detecting an organic analyte in a sample, comprising a lateral flow chromatographic test strip comprising a zone comprising C18.

17. The device of claim 16, wherein the lateral flow chromatographic test strip further comprises a test zone.

18. The device of claim 17, wherein the zone comprising C18 is the test zone, wherein C18 is immobilized in the test zone.

19. The device of claim 17, wherein C18 is located upstream of the test zone, and is bound to a first tag; and a second tag that binds to the first tag is immobilized in the test zone.

20. The device of claim 17, wherein the lateral flow chromatographic test strip further comprises a conjugate zone comprising a conjugate comprising a first binding partner for the organic analyte and a label.

21. The device of claim 16, wherein the organic analyte is a chemical warfare nerve agent and the conjugate is selected from the group consisting of ID8.2 and BE2.

22. A method for detecting an organic analyte in the sample, comprising the steps of:
a) collecting the sample;
b) transferring the sample to a sample analysis device comprising:
i) at least one conjugate zone comprising a conjugate comprising a first binding partner for the organic analyte and a label;
ii) C18; and
iii) a test zone downstream from the conjugate zone;
c) running an assay where the sample encounters the conjugate during the assay; and
d) detecting a presence of the organic analyte in the sample.

23. The method of claim 22, wherein C18 is immobilized in the test zone.

24. The method of claim 22, wherein C18 is located upstream of the test zone, and is bound to a first tag; and a second tag that binds to the first tag is immobilized in the test zone.

25. The method of claim 22, wherein the organic analyte is a chemical warfare nerve agent and the conjugate is selected from the group consisting of ID8.2 and BE2.

26. A device for detecting an analyte in a sample, comprising a detection zone comprising Albegone recombinant protein.

27. The device of claim 26, further comprising an Fclock recombinant protein in a zone upstream from the detection zone.

28. A device for detecting an analyte in a sample, comprising a detection zone and at least one conjugate zone upstream of the detection zone, wherein the conjugate zone comprises Fclock recombinant protein.

29. A method for detecting an analyte in a sample, comprising the step of exposing at least one antibody reagent to Fclock recombinant protein, such that the Fclock recombinant protein binds to an Fc portion of the antibody.

30. A device for detecting a chemical warfare nerve agent, comprising polylysine in a running buffer for the device or on a lateral flow chromatographic test strip.

31. A method of detecting a chemical warfare nerve agent in a sample, comprising the step of exposing the sample to polylysine.

32. The method of claim 31, wherein the sample is exposed to polylysine in a running buffer of an assay.

33. The method of claim 31, wherein the sample is exposed to polylysine on a lateral flow chromatographic test strip.
